(19) United States
(12) Patent Application Publication
(10) Pub. No.: US 2002/0182649 A1
(43) Pub. Date: Dec. 5, 2002

(54) Methods for protein identification, characterization and sequencing by tandem mass spectrometry

(75) Inventors: Scot R. Weinberger, Montara, CA (US); Huw A. Davies, Epsom Downs (GB); Ning Tang, Dublin, CA (US)

Correspondence Address: FISH & NEAVE, 1251 AVENUE OF THE AMERICAS, 50TH FLOOR, NEW YORK, NY, 10020-1105, US

(73) Assignee: Ciphergen Biosystems, Inc., Fremont, CA (US)

(21) Appl. No.: 10/066,359

(22) Filed: Jan. 31, 2002

Related U.S. Application Data
(60) Provisional application No. 60/265,996, filed on Feb. 1, 2001.
(60) Provisional application No. 60/283,817, filed on Apr. 13, 2001.

(51) Int. Cl. G01N 33/53; G01N 33/542; C12P 21/06

(52) U.S. Cl. 435/7.9; 435/68.1

(57) ABSTRACT

Presented are novel apparatus and methods for protein characterization, identification, and sequencing using affinity capture laser desorption/ionization tandem mass spectrometry.

### CROSS REFERENCE TO RELATED APPLICATIONS

This application claims the benefit of the filing dates of provisional application No. 60/283,817, filed Apr. 13, 2001, and No. 60/265,996, filed Feb. 1, 2001, the disclosures of which are incorporated herein by reference in their entireties.

### FIELD OF THE INVENTION

This invention is in the field of chemical and biochemical analysis, and relates particularly to apparatus and methods for improved identification, characterization and sequencing of protein analytes by tandem mass spectrometry.

### BACKGROUND OF THE INVENTION

The advent of electrospray ionization (ESI) and matrix-assisted laser desorption/ionization (MALDI) techniques, coupled with improved performance and lower cost of mass analyzers, has in the past decade allowed mass spectrometry (MS) to take a place among standard analytical tools in the study of biologically relevant macromolecules, including proteins purified from complex biological systems.

For example, in a technique known as peptide mass fingerprinting, mass spectrometry is used to identify proteins purified from biological samples. Identification is effected by matching the mass spectrum of proteolytic fragments of the purified protein with masses predicted from primary sequences prior-accessioned into a database. Roepstorff, The Analyst 117:299-303 (1992); Pappin et al., Curr. Biol. 3(6):327-332 (1993); Mann et al., Biol. Mass Spectrom. 22:338-345 (1993); Yates et al., Anal. Biochem. 213:397-408 (1993); Henzel et al., Proc. Natl. Acad. Sci. USA 90:5011-5015 (1993); James et al., Biochem. Biophys. Res. Commun. 195:58-64 (1993).

Similar database-mining approaches have been developed that use fragment mass spectra obtained from collision induced dissociation (CID) or MALDI post-source decay (PSD) to identify purified proteins. Eng et al., J. Am. Soc. Mass. Spectrom. 5:976-989 (1994)); Griffin et al., Rapid Commun. Mass Spectrom. 9:1546-1551 (1995); Yates et al., U.S. Pat. Nos. 5,538,897 and 6,017,693; Mann et al., Anal. Chem. 66:4390-4399 (1994).

Mass spectrometric techniques have also been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

Software resources that facilitate interpretation of protein mass spectra and mining of public domain sequence databases are now readily accessible on the internet to facilitate protein identification. Among these are Protein Prospector (http://www.prospector.ucsf/edu), PROWL (http://www.proteometrics.com ), and the Mascot Search Engine (Matrix Science Ltd., London, UK, www.matrixscience.com).

Although highly accurate mass assignment provides useful information—facilitating identification of purified protein by the above-described techniques, for example—such information is nonetheless limited. Significant additional analytical power would be unleashed by combining MS analysis with enzymatic and/or chemical modification of target proteins, enabling the elucidation of structural components, post-translational modifications, and furthering protein identification.

Furthermore, complex biological materials—such as blood, sera, plasma, lymph, interstitial fluid, urine, exudates, whole cells, cell lysates and cellular secretion products—typically contain hundreds of biological molecules, plus organic and inorganic salts, which precludes direct mass spectrometry analysis. Thus, significant sample preparation and purification steps are typically necessary prior to MS investigation.

Classical methods of sample purification, such as liquid chromatography (ion exchange, size exclusion, affinity, and reverse phase chromatography), membrane dialysis, centrifugation, immunoprecipitation, and electrophoresis, typically demand a large quantity of starting sample. Even when such quantities of sample are available, minor components tend to become lost in these purification processes, which suffer from analyte loss due to non-specific binding and dilution effects. The methods are also often quite labor intensive.

Thus, there is a clear need for methods and apparatus that facilitate mass spectrometric detection of both major and minor proteins present in heterogeneous samples without requiring extensive prior fluid phase purification. There is further need for an MS platform that allows not only facile sample purification, but also permits serial and parallel sample modification approaches prior to mass spectrometric analysis.

These needs have been met, in part, by the development of affinity capture laser desorption ionization approaches. Hutchens et al., Rapid Commun. Mass Spectrom. 7: 576-580 (1993); U.S. Pat. Nos. 5,719,060, 5,894,063, 6,020,208, and 6,027,942. This new strategy for MS analysis of macromolecules uses novel laser desorption ionization probes that have an affinity reagent on at least one surface. The affinity reagent adsorbs desired analytes from heterogeneous samples, concentrating them on the probe surface in a form suitable for subsequent laser desorption ionization. The coupling of adsorption and desorption of analyte obviates off-line purification approaches, permitting analysis of smaller initial samples and further facilitating sample modification approaches directly on the probe surface prior to mass spectrometric analysis.

The affinity capture laser desorption ionization approach has allowed mass spectrometry to be enlisted in the performance of numerous classic bioanalytical techniques, including immunoassay, Nelson et al., Anal. Chem. 67: 1153-1158 (1995), and affinity chromatography, Brockman et al., Anal. Chem. 67: 4581-4585 (1995). The affinity capture laser desorption ionization approach has been applied not only to the study of peptides and proteins, Hutchens et al., Rapid Commun. Mass Spectrom. 7:576-580 (1993); Mouradian et al., J. Amer. Chem. Soc. 118: 8639-8645 (1996); Nelson et al., Rapid Commun. Mass. Spectrom. 9: 1380-1385 (1995); Nelson et al., J. Molec. Recognition 12: 77-93 (1999).; Brockman et al., J. Mass Spectrom. 33: 1141-1147 (1998); Yip et al., J. Biol. Chem. 271: 32825-33 (1996), but also to oligonucleotides, Jurinke et al., Anal. Chem. 69:904-910 (1997); Tang et al., Nucl. Acids Res. 23: 3126-3131 (1995); Liu et al., Anal. Chem. 67: 3482-90 (1995), bacteria, Bundy et al., Anal. Chem. 71: 1460-1463 (1999), and small molecules, Wei et al., Nature 399:243-246 (1999). At the commercial level, affinity capture laser desorption ionization is embodied in Ciphergen's ProteinChip® Systems (Ciphergen Biosystems, Inc. Fremont, Calif., USA).

Although the affinity capture laser desorption ionization technique has solved significant problems in the art, difficulties remain.

For example, when this approach is applied to capture proteins from biological samples, it is common to see about one picomole of total protein captured and available for subsequent analysis. Typically, affinity capture on chromatographic surface affinity capture probes does not result in complete purification. Additionally, the digestion efficiency seen for solid phase extracted samples, as compared to digests performed in free solution or the denaturing environment of 2-D gels, is poor. Thus, if about 50% were the protein of interest, and one were successful in digesting about 10% of this protein, at best only about 50 femtomole of some peptides would be available for detection.

Additionally, using virtual tryptic digests of bovine fetuin in database mining experiments, it has been demonstrated that even with an extreme accuracy of 1.0 ppm (a level not currently achievable by most MS techniques), a poor confidence protein ID match is achieved with a single peptide mass when searching against this complex, eukaryotic genome. For two peptides, low confidence results are achieved as well. Only after three peptides are submitted are confident results returned for mass assignments of less than 300 ppm error. In this case, most devices would require internal standard calibration. However, with five or more peptides, no further confidence is afforded with mass accuracies that are better than 1000 ppm error.

Furthermore, when multiple proteins are simultaneously digested, a heterogeneous peptide pool is created and successful database mining requires not only extreme accuracy, but in many instances primary sequence information as well. Although tandem MS/MS approaches have demonstrated significant utility in providing primary sequence information, Biemann et al., Acc. Chem. Res. 27: 370-378 (1994); Spengler et al., Rapid Commun. Mass Spectrom. 1991, 5:198-202 (1991); Spengler et al., Rapid Commun. Mass Spectrom. 6:105-108 (1992); Yates et al., Anal. Chem. 67:1426-1436 (1995); Kaufman et al., Rapid Commun. Mass. Spectrom. 7:902-910 (1993); Kaufman et al., Intern. J. Mass Spectrom. Ion Processes 131:355-385 (1994), the admixture of protein cleavage products from multiple proteins often requires additional off-line purification prior to tandem MS sequence analysis.

Furthermore, until recently the only MS/MS approach available for laser desorption based analyses was post source decay analysis (PSD). While PSD is capable of providing reasonable sequence information for picomole levels of peptides, the overall efficiency of this fragmentation process is low; when combined with the poor mass accuracy and sensitivity often demonstrated during this approach, its applicability to analysis of low abundance proteins often found on affinity capture laser desorption ionization probes has been greatly limited.

There is, therefore, a need for apparatus and methods that would increase the sensitivity and mass accuracy of affinity capture laser desorption mass spectrometry. There is a need for methods and apparatus that would increase on-probe digestion efficiency and that would permit peptides generated by digest of inhomogeneous mixtures of proteins readily to be resolved. There is a need for apparatus and methods that would increase the efficiency of affinity capture laser desorption tandem mass spectrometric analysis.

Recently, a laser desorption ionization quadrupole time-of-flight mass spectrometer (LDI Qq-TOF) has been developed that is capable of performing collision induced dissociation (CID) MS/MS analysis. Krutchinksy et al., Rapid Commun. Mass Spectrom. 12: 508-518 (1998).

### SUMMARY OF THE INVENTION

It is an object of the present invention to provide apparatus for affinity capture probe laser desorption ionization mass spectrometry that has increased sensitivity, mass accuracy, and mass resolution as compared to existing affinity capture laser desorption ionization mass spectrometers. It is a further object of the present invention to provide apparatus for affinity capture probe laser desorption ionization mass spectrometry that adds MS/MS capability. It is a further object of the present invention to provide novel methods of biomolecule analysis, particularly protein analysis, that exploit these improved analytical capabilities.

The present invention meets these and other objects and needs in the art by providing, in a first aspect, an analytical instrument.

The analytical instrument of the present invention comprises a laser desorption ionization source, an affinity capture probe interface, and a tandem mass spectrometer, in which the affinity capture probe interface is capable of engaging an affinity capture probe and positioning the probe so that it can be interrogated by the laser desorption source while in communication with the tandem mass spectrometer, thus permitting ions desorbed from the probe to enter the mass spectrometer.

Typically, the laser desorption ionization source comprises a laser excitation source and a laser optical train; the laser optical train functions to transmit excited photons from the laser excitation source to the probe interface. In such embodiments, the laser optical train typically delivers about 20-1000 microjoules of energy per square millimeter of interrogated probe surface.

The laser excitation source is selected from the group consisting of a chopped continuous laser and a pulsed laser, and in various embodiments is selected from the group consisting of a nitrogen laser, a Nd:YAG laser, an erbium:YAG laser, and a CO₂ laser. In a presently preferred embodiment, the laser excitation source is a pulsed nitrogen laser.

In one set of embodiments, the laser optical train comprises optical components selected from the group consisting of lenses, mirrors, prisms, attenuators, and beam splitters.

In an alternative set of embodiments, the laser optical train comprises an optical fiber having an input end and an output end, and the laser excitation source is coupled to the optical fiber input end.

In some of the optical fiber laser optical train embodiments, the laser optical train further comprises an optical attenuator. The attenuator can be positioned between the laser excitation source and the input end of the optical fiber, can serve to couple the laser excitation source to the input end of the optical fiber, or can be positioned between the optical fiber output end and the probe.

In certain of the optical fiber optical train embodiments, the optical fiber output end has a maximum diameter between about 200-400 μm and the input end has a diameter of between about 400 to 1200 μm.

The analytical instrument can also include probe viewing optics, to permit the probe to be visualized after its engagement in the probe interface.

In certain embodiments, the laser optical train can include a laser coupler that couples the laser excitation source to the optical fiber input end. As noted above, the coupler can serve as an optical attenuator. In other embodiments, the coupler can serve to promote visualization of the probe after its engagement in the probe interface.

In certain of these latter embodiments, either the coupler or the fiber is bifurcated and splits off a fraction of energy from the laser excitation source. Alternatively, such bifurcation can allow introduction of visible light to illuminate the desorption locus.

Where visualization optics are included in the optical train, or where a fiber-containing laser optical train includes a bifurcation or trifurcation, the analytical instrument can further comprise a CCD camera positioned to detect light reflected from the probe.

In typical embodiments, the affinity capture probe interface comprises a probe holder which is capable of reversibly engaging the affinity capture probe. The interface also typically comprises a probe introduction port which is itself capable of reversibly engaging the probe holder.

In typical embodiments, the probe interface further comprises a probe position actuator assembly and an interface ion collection system. When the probe holder is engaged in the introduction port, it is placed in contact with the probe position actuator; the probe position actuator, in turn, is capable of movably positioning the probe holder (typically with its engaged probe) with respect both to the laser ionization source (typically, with respect to the laser optical train) and to the ion collection system. In typical embodiments, the actuator is capable of translationally and rotationally positioning the probe holder.

The probe interface typically also comprises a vacuum evacuation system coupled to the probe introduction port, which allows the probe to be interrogated by the laser desorption ionization source at subatmospheric pressures.

The analytical instrument of the present invention comprises a tandem mass spectrometer which, in various embodiments, is selected from the group consisting of a QqTOF MS, an ion trap MS, an ion trap TOF MS, a TOF-TOF MS, and a Fourier transform ion cyclotron resonance MS. Presently preferred for use in the analytical instrument of the present invention is a QqTOF MS.

In preferred embodiments, the tandem mass spectrometer is a QqTOF MS and the laser excitation source is a pulsed nitrogen laser, laser fluence at the probe is about 2 to 4 times the minimum desorption threshold, and the tandem mass spectrometer has an external standard mass accuracy of about 20-50 ppm.

The analytical instrument of the present invention is designed to engage an affinity capture laser desorption ionization probe. Accordingly, any of the above-described embodiments can include an affinity capture probe engaged in the affinity capture probe interface.

The affinity capture probe in these embodiments will typically have at least one sample adsorption surface positioned in interrogatable relationship to the laser source, the sample adsorption surface selected from the group consisting of chromatographic adsorption surfaces and biomolecule affinity surfaces. Typically, such chromatographic adsorption surface is selected from the group consisting of reverse phase, anion exchange, cation exchange, immobilized metal affinity capture and mixed-mode surfaces and the biomolecule of the biomolecule affinity surfaces is selected from the group consisting of antibodies, receptors, nucleic acids, lectins, enzymes, biotin, avidin, streptavidin, Staph protein A and Staph protein G.

The affinity capture laser desorption ionization probe can have a plurality of separately addressable sample adsorption surfaces that can be positioned in interrogatable relationship to the laser source and can include at least two different such adsorption surfaces.

In other embodiments, the analytical instrument of the present invention includes a digital computer interfaced with a detector of the tandem mass spectrometer. In some embodiments, the instrument can also further include a software program executable by the digital computer, either local to the computer or communicably accessible to the computer. The software program in such embodiments can be capable of controlling the laser desorption ionization source, or of controlling at least one aspect of data acquisition by the tandem mass spectrometer, or of performing at least one analytical routine on data acquired by the tandem mass spectrometer, or any subset of these functions.

In a second aspect, the invention provides a method for analyzing a protein analyte present as a plurality of cleavage products in admixture with cleavage products of other proteins.

In general, the method of this aspect of the invention comprises the steps of (a) capturing a plurality of cleavage products from the mixture by adsorption to an affinity capture probe, the plurality of adsorbed cleavage products including at least one cleavage product of the protein analyte; (b) washing the probe at least once with a first eluant for a time and under conditions sufficient to decrease the complexity of the plurality of adsorbed cleavage products, the adsorbed cleavage products of reduced complexity including at least one cleavage product of the protein analyte; and then (c) characterizing the at least one cleavage product of the protein analyte with a tandem mass spectrometer measurement.

The tandem mass spectrometric characterization of the cleavage product provides an analysis of the protein analyte. Optionally, the method includes an antecedent step of cleaving the proteins in the mixture into cleavage products using a proteolytic agent.

The wash step serves to decrease the complexity of the mixture of cleavage products, facilitating the subsequent tandem mass spectrometric analysis. In some embodiments, after washing with the first eluant and before performing tandem mass spectrometric characterization, at least one iteration of a second wash step is performed. The second wash is done with a second eluant which differs in at least one elution characteristic from the first eluant, for a time and under conditions sufficient further to decrease the complexity of the plurality of adsorbed protein cleavage products, the adsorbed cleavage products of further reduced complexity including at least one cleavage product of the protein analyte.

Depending upon the nature of the affinity capture probe, in certain embodiments of the method energy absorbing molecules are applied to the probe after washing, and before tandem mass spectrometric analysis. The energy absorbing molecules are applied so as to contact the protein cleavage products.

Typically, the tandem mass spectrometric characterization includes the following steps: (i) desorbing and ionizing the protein cleavage products from the probe, thus generating parent peptide ions corresponding to the cleavage products; (ii) selecting a desired parent peptide ion in a first phase of mass spectrometry; (iii) fragmenting the selected parent peptide ion in the gas phase into fragment ions; and then (iv) measuring the mass spectrum of the fragment ions of the selected parent peptide ion in a second phase of mass spectrometry. In the embodiment of the method practiced with the QqTOF instrument of the present invention, the gas phase fragmenting is effected by collision induced dissociation (CID).

In certain embodiments of the method in which identification of the protein analyte is desired, the method can further comprise determining at least a portion of the amino acid sequence of the protein analyte.

The sequence information is typically obtained by calculating differences in masses among fragment ions of a particular fragmentation series represented in the fragment ion mass spectrum. Identification can be furthered by using the partial sequence information to obtain a protein identity candidate based upon the closeness-of-fit calculated between the amino acid sequence predicted by mass spectrometry and sequences prior-accessioned into a sequence database. In some embodiments, the closeness-of-fit is calculated additionally from the mass of the parent peptide ion and optionally from the genus or species of protein analyte origin.

The likelihood that the identity candidate is the same as the protein analyte can be assessed by comparing (i) the mass measured for the selected parent peptide ion to (ii) the masses predicted for cleavage products that would be generated by cleaving the identity candidate with the proteolytic agent, a match as between a predicted mass and the measured mass indicating increased likelihood that the identity candidate is the same as the protein analyte.

Further validation of the protein identity candidate can be obtained comparing the predicted cleavage product masses to masses measured for cleavage products desorbed from the probe other than the cleavage product characterized by fragmentation and a second phase of mass spectrometry; in this embodiment, additional matches as between predicted and measured masses indicates an increased likelihood that the identity candidate is the same as the protein analyte.

Conversely, when the predicted mass and the measured mass do not match, steps of the method can be repeated on a desorbed cleavage product other than the characterized cleavage product.

Sequence data is not required for protein identification.

Thus, in other embodiments, at least one protein identity candidate is determined for the protein analyte based instead upon the closeness-of-fit calculated between the fragment ion mass spectrum and mass spectra predicted from sequences prior-accessioned into a sequence database. In some embodiments, the closeness-of-fit is calculated additionally from the mass of the parent peptide ion and optionally from the genus or species of protein analyte origin.

The likelihood that the identity candidate is the same as the protein analyte can be assessed by comparing (i) the mass measured for the selected parent peptide ion to (ii) the masses predicted for cleavage products that would be generated by cleaving the identity candidate with the proteolytic agent, a match as between a predicted mass and the measured mass indicating increased likelihood that the identity candidate is the same as the protein analyte.

Further validation of the protein identity candidate can be obtained comparing the predicted cleavage product masses to masses measured for cleavage products desorbed from the probe other than the cleavage product characterized by fragmentation and a second phase of mass spectrometry; in this embodiment, additional matches as between predicted and measured masses indicates an increased likelihood that the identity candidate is the same as the protein analyte.

Conversely, when the predicted mass and the measured mass do not match, steps of the method can be repeated on a desorbed cleavage product (parent peptide ion) other than the characterized cleavage product.

The various embodiments of the method of this aspect of the invention can be performed using an analytical instrument comprising a variety of tandem mass spectrometers, such as QqTOF mass spectrometer, ion trap mass spectrometer, ion trap time-of-flight (TOF) mass spectrometer, time-of-flight time-of-flight (TOF-TOF) mass spectrometer, or a Fourier transform ion cyclotron resonance mass spectrometer. As noted above, analytical instruments comprising a QqTOF tandem mass spectrometer present advantages.

In the various embodiments of the method of this aspect of the invention, the affinity capture probe can have a chromatographic adsorption surface, such as a reverse phase surface, anion exchange surface, cation exchange surface, immobilized metal affinity capture surface and mixed-mode surface, or can have a biomolecule affinity surface.

Typically, in the methods of this aspect of the invention, the protein mixture is, or is derived from, a biologic sample, such as blood, blood fraction, lymph, urine, cerebrospinal fluid, synovial fluid, milk, saliva, vitreous humor, aqueous humor, mucus or semen. The biological sample can also usefully be a cell lysate.

In a third aspect, the present invention provides a method for analyzing a protein analyte present within a mixture of proteins.

The method comprises the following steps: (a) capturing at least the protein analyte from the mixture by adsorption to an affinity capture probe; (b) cleaving proteins adsorbed to the affinity capture probe into protein cleavage products using a proteolytic agent; (c) washing the probe at least once with a first eluant for a time and under conditions sufficient to increase the relative concentration among protein cleavage products adsorbed to the probe of at least one cleavage product of the protein analyte; and then (d) characterizing the at least one cleavage product of the protein analyte with a tandem mass spectrometer measurement. The tandem mass spectrometric characterization of the cleavage product provides an analysis of the protein analyte.

The wash step serves to decrease the complexity of the mixture of cleavage products, and can increase the collective sequence coverage of the detected peptides, facilitating the subsequent tandem mass spectrometric analysis. In some embodiments, after washing with the first eluant and before performing tandem mass spectrometric characterization, at least one iteration of a second wash step is performed. The second wash is done with a second eluant which differs in at least one elution characteristic from the first eluant, for a time and under conditions sufficient further to increase the relative concentration among protein cleavage products adsorbed to the probe of at least one cleavage product of the protein analyte.

Depending upon the nature of the affinity capture probe, in certain embodiments of the method energy absorbing molecules are applied to the probe after washing, and before tandem mass spectrometric analysis. The energy absorbing molecules are applied so as to contact the protein cleavage products and incorporate the protein cleavage products into the matrix crystal, thus allowing ultimate detection using a laser desorption ionization source.

Typically, the tandem mass spectrometric characterization includes the following steps: (i) desorbing and ionizing the protein cleavage products from the probe, thus generating parent peptide ions corresponding to the cleavage products; (ii) selecting a desired parent peptide ion in a first phase of mass spectrometry; (iii) fragmenting the selected parent peptide ion in the gas phase into fragment ions; and then (iv) measuring the mass spectrum of the fragment ions of the selected parent peptide ion in a second phase of mass spectrometry. In the embodiment of the method practiced with the QqTOF instrument of the present invention, the gas phase fragmenting is effected by collision induced dissociation (CID).

In certain embodiments of the method in which identification of the protein analyte is desired, the method can further comprise determining at least a portion of the amino acid sequence of the protein analyte.

The sequence information is typically obtained by calculating differences in masses among fragment ions of a particular fragment series represented in the fragment ion mass spectrum. Identification can be furthered by using the partial sequence information to obtain a protein identity candidate based upon the closeness-of-fit calculated between the amino acid sequence predicted by mass spectrometry and sequences prior-accessioned into a sequence database. In some embodiments, the closeness-of-fit is calculated additionally from the mass of the parent peptide ion and optionally from the genus or species of protein analyte origin.

The likelihood that the identity candidate is the same as the protein analyte can be assessed by comparing (i) the mass measured for the selected parent peptide ion to (ii) the masses predicted for cleavage products that would be generated by cleaving the identity candidate with the proteolytic agent, a match as between a predicted mass and the measured mass indicating increased likelihood that the identity candidate is the same as the protein analyte.

Further validation of the protein identity candidate can be obtained comparing the predicted cleavage product masses to masses measured for cleavage products desorbed from the probe other than the cleavage product characterized by fragmentation and a second phase of mass spectrometry; in this embodiment, additional matches as between predicted and measured masses indicates an increased likelihood that the identity candidate is the same as the protein analyte.

Conversely, when the predicted mass and the measured mass do not match, steps of the method can be repeated on a desorbed cleavage product other than the characterized cleavage product.

Sequence data is not required for protein identification.

Thus, in some embodiments, at least one protein identity candidate is determined for the protein analyte based instead upon the closeness-of-fit calculated between the fragment ion mass spectrum and mass spectra predicted from sequences prior-accessioned into a sequence database. In some embodiments, the closeness-of-fit is calculated additionally from the mass of the parent peptide ion and optionally from the genus or species of protein analyte origin.

The likelihood that the identity candidate is the same as the protein analyte can be assessed by comparing (i) the mass measured for the selected parent peptide ion to (ii) the masses predicted for cleavage products that would be generated by cleaving the identity candidate with the proteolytic agent, a match as between a predicted mass and the measured mass indicating increased likelihood that the identity candidate is the same as the protein analyte.

Further validation of the protein identity candidate can be obtained comparing the predicted cleavage product masses to masses measured for cleavage products desorbed from the probe other than the cleavage product characterized by fragmentation and a second phase of mass spectrometry; in this embodiment, additional matches as between predicted and measured masses indicates an increased likelihood that the identity candidate is the same as the protein analyte.

Conversely, when the predicted mass and the measured mass do not match, steps of the method can be repeated on a desorbed cleavage product other than the characterized cleavage product.

The various embodiments of the method of this aspect of the invention can be performed using an analytical instrument comprising a variety of tandem mass spectrometers, such as QqTOF mass spectrometer, ion trap mass spectrometer, ion trap time-of-flight (TOF) mass spectrometer, time-of-flight time-of-flight (TOF-TOF) mass spectrometer, or a Fourier transform ion cyclotron resonance mass spectrometer. As noted above, analytical instruments comprising a QqTOF tandem mass spectrometer present advantages.

In the various embodiments of the method of this aspect of the invention, the affinity capture probe can have a chromatographic adsorption surface, such as a reverse phase surface, anion exchange surface, cation exchange surface, immobilized metal affinity capture surface and mixed-mode surface, or can have a biomolecule affinity surface.

Typically, in the methods of this aspect of the invention, the protein mixture is, or is derived from, a biologic sample, such as blood, blood fraction, lymph, urine, cerebrospinal fluid, synovial fluid, milk, saliva, vitreous humor, aqueous humor, mucus or semen. The biological sample can also usefully be a cell lysate.

In a fourth aspect, the invention provides a method for analyzing at least one test protein.

The method comprises (a) capturing the test protein or proteins on an affinity capture probe (“protein biochip”), (b) generating protein cleavage products of the test protein(s) on the protein biochip using a proteolytic agent; and (c) analyzing at least one protein cleavage product with a tandem mass spectrometer. In contrast to the methods of the third aspect of this invention, wash of the probe prior to analysis is not required and can be omitted.

In the methods of this aspect of the invention, the analyzing step comprises (i) desorbing the protein cleavage products from the protein biochip into gas phase to generate corresponding parent peptide ions, (ii) selecting a parent peptide ion for subsequent fragmentation with a first mass spectrometer, (iii) fragmenting the selected parent peptide ion under selected fragmentation conditions in the gas phase to produce product ion fragments and (iv) generating a mass spectrum of the product ion fragments. In this fashion, the mass spectrum provides an analysis of the test proteins.

In certain embodiments of this aspect of the invention, the method further includes an additional step (d), determining at least one protein identity candidate for the test protein.

In one approach, the protein identity candidate is identified by submitting the mass spectrum to a protein database mining protocol which identifies at least one protein identity candidate for the test protein in the database based on a measure of closeness-of-fit between the mass spectrum and theoretical mass spectra of proteins in the database. In particular of these embodiments, step (d) further comprises submitting the mass of the test protein and the species of origin of the test protein to the protocol.

In another approach, the protein identity candidate is identified after at least partial de novo MS/MS sequence determination of the peptide selected in the first phase of MS analysis. The partial sequence is then used to query sequence databases to identify related sequences prior accessioned into the database. Optionally, the species or genus of protein origin can be used to facilitate or filter the query, as can the mass of the selected peptide and, if known, the mass of the uncleaved and unfragmented protein analyte.

The two approaches are not mutually exclusive and can be practiced serially or in parallel.

In various embodiments that can be practiced with either approach to identifying the protein identity candidate, the method further comprises (e) comparing the identity candidate to the test protein by: (i) generating a mass spectrum of the protein cleavage products of (b); (ii) submitting the mass spectrum of the protein cleavage products to a computer protocol that determines a measure of closeness-of-fit between the theoretical mass spectrum of cleavage products of the identity candidate predicted to be generated by using the proteolytic agent, and the mass spectrum of the protein cleavage products, whereby the measure indicates protein cleavage products on the protein biochip that correspond to the test protein.

Yet other embodiments of the method include the further steps of (f), repeating step (c) wherein the selected parent peptide ion does not correspond to a protein cleavage product predicted from the identity candidate; and then (g) repeating (d) for the selected parent peptide ion of (f).

In this fourth aspect of the invention, as well as in the second and third aspects, the protein analyte (the test protein) can be a protein that is differentially expressed as between first and second biological samples. In some of these embodiments, the first and second biological samples are derived from normal and pathological sources.

In a fifth aspect, the invention provides a method of characterizing binding interactions between a first and second molecular binding partner.

In this aspect, the method comprises binding a second binding partner to a first binding partner, where the first binding partner is immobilized to a surface of a laser desorption ionization probe; fragmenting the second binding partner; and then detecting at least one of the fragments by a tandem mass spectrometer measurement, whereby the mass spectrum of the detected fragments characterizes the binding interactions.

In certain embodiments of this aspect of the invention, the first binding partner is first immobilized to a surface of an affinity capture probe before the second binding partner is bound to the first binding partner.

Such immobilizing can be by direct binding of the first partner to the affinity capture probe, such as a covalent bonding. Typical covalent bonding embodiments include covalent bonding between an amine of the first binding partner and a carbonyldiimidazole moiety of the probe surface and between an amino or thiol group of the first binding partner and an epoxy group of the probe surface.

The immobilizing can also be by direct noncovalent bonding, such as a coordinate or dative bonding between the first binding partner and a metal, such as gold or platinum, of the probe surface. The immobilizing can also be by interaction of the first binding partner to a chromatographic adsorption surface selected from the group consisting of reverse phase, anion exchange, cation exchange, immobilized metal affinity capture and mixed-mode surfaces.

Alternatively, the immobilizing can be indirect. In some embodiments, the indirect binding can be covalent, albeit indirect. In certain of these latter embodiments, the first binding partner can be immobilized by covalent bonding through a linker, such as a cleavable linker. Indirect immobilization can also be noncovalent, such as immobilization to the probe via a biotin/avidin, biotin/streptavidin interaction.

In this aspect of the invention, the first molecular binding partner can be selected from the group consisting of protein, nucleic acid, carbohydrate, and lipid. Typically, the first binding partner will be a protein, which can be a naturally occurring protein from an organism selected from the group consisting of multicellular eukaryote, single cell eukaryote, prokaryote, and virus, or can be a nonnaturally occurring protein, such as a recombinant fusion protein.

In embodiments in which the first binding partner is a protein, the protein can be selected from the group consisting of antibody, receptor, transcription factor, cytoskeletal protein, cell cycle protein, and ribosomal protein, among others.

Binding of the second binding partner to the immobilized first binding partner is, in typical embodiments, effected by contacting the first binding partner with a biologic sample; the sample can be a fluid selected from the group consisting of blood, lymph, urine, cerebrospinal fluid, synovial fluid, milk, saliva, vitreous humor, aqueous humor, mucus and semen, or a cell lysate, or some sample in another form.

In various embodiments, including embodiments in which the first binding partner is a protein, the second binding partner can be a protein. Alternatively, the second binding partner can be a compound present in a combinatorial library, where binding of the second binding partner to the first binding partner is effected by contacting the first binding partner with an aliquot of a chemically synthesized combinatorial library. In yet other alternatives, the second binding partner can be a component of biologically displayed combinatorial library, such as a phage-displayed library.

In certain typical embodiments, fragmenting is effected by contacting the second binding partner with an enzyme; where the second binding partner is a protein, the enzyme is typically a specific endoprotease, such as trypsin, Glu-C (V8) protease, endoproteinase Arg-C (serine protease), endoproteinase Arg-C (cysteine protease), Asn-N protease, and Lys-C protease. The protease can also be one of quasi-specificity such as pepsin, thermolysin, papain, subtilisin, and pronazse. Alternatively, fragmenting can be effected by contacting the second binding partner with a liquid phase chemical, such as CNBr or several organic or inorganic acids capable of performing acid catalyzed hydrolysis of a polypeptide chain.

In some embodiments, the method further comprises, after binding of the second binding partner to the first binding partner, and before fragmenting the second binding partner, of denaturing the second binding partner.

In various embodiments, the method further comprises the step, after fragmenting the second binding partner, of washing the probe with a first eluant, and, at times, a second eluant, the second eluant differing from the first eluant in at least one elution characteristic, such as pH, ionic strength, detergent strength, and hydrophobicity.

In typical embodiments, the method further comprises, after fragmenting and before detecting the fragments of the second binding partner, the step of applying energy absorbing molecules to the probe. In preferred embodiments, the probe is then engaged in the affinity capture probe interface of the analytical instrument of the present invention, and fragments of the second binding partner ionized and desorbed from the probe using the instrument's laser source.

The instrument can be used to make several types of useful measurements in this method, including a measurement of all ion masses, a measurement of masses of a subset of fragments, and a single ion monitoring measurement.

Usefully, embodiments of the method include the step, after mass spectrometric measurement of fragments of the second binding partner, of comparing the fragment measurements with those predicted by applying cleavage rules of the fragmenting enzyme to the primary amino acid sequence of the second binding partner, whereby such comparison characterizes the intermolecular interactions.

If the identity of the second binding partner is not known, the method can further comprise, before such comparison, identifying the second binding partner through ms/ms analysis. Such MS/MS analysis can include the steps of mass spectrometrically selecting a first fragment of the second binding partner; dissociating the second binding partner first fragment in the gas phase; measuring the fragment spectrum of the second binding partner first fragment, and then comparing the fragment spectrum to fragment spectra predicted from amino acid sequence data prior-accessioned in a database. The amino acid sequence data can be selected from the group consisting of empiric and predicted data, and the dissociating, in typically embodiments, is collision induced dissociation.

In some embodiments of the method, the first binding partner is selected from the group consisting of an antibody, a T cell receptor, and an MHC molecule. In other embodiments, the first binding partner is a receptor and the second binding partner is selected from the group consisting of an agonist of the receptor, a partial agonist of the receptor, an antagonist of the receptor, and a partial antagonist of the receptor. In other embodiments, the first binding partner is a glycoprotein receptor and the second binding partner is a lectin.

In a sixth aspect, the invention provides a method of detecting an analyte, the method comprising engaging an affinity capture probe in the affinity capture probe interface of the analytical instrument of the present invention, the affinity capture probe having an analyte bound thereto; desorbing and ionizing the analyte or fragments thereof from the probe using the instrument's laser source; and then detecting the analyte by a tandem mass spectrometer measurement on the desorbed ions.

In this aspect, the method can further comprise, after the desorbing and ionizing step and before detecting, effecting collision induced dissociation of the desorbed ions. Before such dissociation, in some embodiments a subset of ions can be selected for collisional dissociation.

In other embodiments, the antecedent step can be performed of adsorbing analyte to the probe, and in yet other embodiments, a step can be performed after adsorbing analyte and prior to engaging the probe in the probe interface, of adherently contacting the probe and the analyte with energy absorbing molecules.

In a yet further aspect, the invention provides a method for detecting a target protein in a sample. The method comprises (a) capturing the target protein on an affinity capture probe; generating protein cleavage products of the target protein on the affinity capture probe using a proteolytic agent; (c) detecting the protein cleavage products by mass spectrometry, and (d) correlating one or more detected protein cleavage products with one or more prior-determined protein fragment markers of the target protein, whereby the correlation detects the target protein. Typically, the mass spectral detection of protein cleavage products comprises desorbing the protein cleavage products from the affinity capture probe into the gas phase to generate corresponding ion proteins and generating a mass spectrum of the desorbed ion proteins.

The protein fragment markers can be determined as follows: (i) capturing the target protein on an affinity capture probe; (ii) generating protein cleavage products on the affinity capture probe using a proteolytic agent; (iii) analyzing at least one protein cleavage product with a tandem mass spectrometer; (iv) identifying at least one protein fragment marker of the test protein from among the candidate protein cleavage products, whereby a correspondence indicates that the protein cleavage product is a protein fragment marker of the test protein.

Typically, step (iii), analyzing at least one protein cleavage product with a tandem mass spectrometer, comprises: (1) desorbing the protein cleavage products from the affinity capture probe into gas phase to generate corresponding parent ion peptides, (2) selecting a parent ion peptide for subsequent fragmentation with a first mass spectrometer, (3) fragmenting the selected parent ion peptide under selected fragmentation conditions in the gas phase to produce product ion fragments and (4) generating a mass spectrum of the product ion fragments with a second mass spectrometer.

Typically, step (iv), identifying at least one protein fragment marker of the test protein from among the candidate protein cleavage products by: (1) submitting at least one mass spectrum to a protein database mining protocol which identifies at least one protein identity candidate for the test protein in the database based on a measure of closeness-of-fit between the mass spectrum and theoretical mass spectra of proteins in the database; and (2) determining whether the identify candidate corresponds to the test protein.

In certain embodiments of the methods of this aspect of the invention, mass spectrometry is laser desorption/ionization mass spectrometry, and in particular, laser desorption/ionization time-of-flight mass spectrometry. Furthermore, in various embodiments the proteolytic agent used in the methods is selected from the group consisting of chemical agents and enzymatic agents.

In a yet further aspect, the invention provides a method for identifying a protein that is differentially displayed between two complex biologic samples. The method comprises: (a) detecting at least one protein that is differentially displayed between two samples with a mass spectrometer; (b) fragmenting proteins in the two samples and detecting protein fragments that are differentially displayed between the two samples with a mass spectrometer; (c) determining the identify of at least one differentially displayed protein fragment with a tandem mass spectrometer; and (d) correlating the identity of the protein fragment with a differentially displayed protein, whereby the correlation identifies a differentially displayed protein.

In certain embodiments of this method, step (a), “detecting”, comprises: (i) capturing proteins from the samples on affinity capture probe; (ii) analyzing the captured proteins from each sample by laser desorption/ionization mass spectrometry; and (iii) comparing the captured proteins in the two samples to identify proteins that are differentially expressed.

In certain embodiments, step (b), “fragmenting and detecting”, comprises: (i) capturing proteins from the samples on affinity capture probes; (ii) generating protein cleavage products on the affinity capture probes using a proteolytic agent; (iii) analyzing the protein cleavage products by laser desorption/ionization mass spectrometry; and (iv) comparing the protein cleavage products in the two samples to identify protein cleavage products that are differentially expressed.

In certain embodiments of the method of this aspect of the invention, step (c), “determining the identity of at least one differentially displayed protein fragment”, comprises: (i) desorbing the protein cleavage products from the protein biochip into gas phase to generate corresponding parent peptide ions, (ii) selecting a parent peptide ion for subsequent fragmentation with a first mass spectrometer, (iii) fragmenting the selected parent peptide ion under selected fragmentation conditions in the gas phase to produce product ion fragments with a second mass spectrometer, (iv) generating a mass spectrum of the product ion fragments; and (v) identifying at least one protein identity candidate fragment marker product by submitting at least one mass spectrum to a protein database mining protocol which identifies at least one protein identity candidate for the differentially displayed protein in the database based on a measure of closeness-of-fit between the mass spectrum and theoretical mass spectra of proteins in the database.

In various embodiments of this aspect of the invention, fragmenting is performed in solution. In other embodiments, fragmenting is performed on the affinity capture probe (“chip”).

Fragmentation can comprise enzymatic fragmentation, including limited enzymatic digestion. Alternatively, fragmenting can comprise chemical fragmentation, including acid hydrolysis.

The differentially displayed protein can be a unique protein. Furthermore, the two samples can be selected from (1) a sample from a healthy source and a sample from a diseased source, (2) a sample from a test model exposed to a toxic compound and a sample from a test model not exposed to the toxic compound or (3) a sample from a subject that responds to a drug and a sample from a subject that does not respond to the drug.

### BRIEF DESCRIPTION OF THE DRAWINGS

The above and other objects and advantages of the present invention will be apparent upon consideration of the following detailed description taken in conjunction with the accompanying drawings, in which like characters refer to like parts throughout, and in which:

FIG. 1 schematizes an embodiment of the analytical instrument of the present invention;

FIG. 2 shows in greater detail the elements of an orthogonal QqTOF tandem mass spectrometer preferred for use in the analytical instrument of the present invention;

FIG. 3 displays the seminal fluid protein profiles of a single BPH and prostate cancer patient;

FIG. 4 shows results of on-probe isolation of one of the upregulated proteins detectable in FIG. 3;

FIG. 5 shows peptides detected by a single phase of MS analysis after the enriched biomarker candidate of FIG. 4 was exposed to in situ digestion using trypsin;

FIG. 6 shows LDI Qq-TOF MS analysis of the same purified protein peptides as shown in FIG. 5;

FIG. 7 shows MS/MS results from the analytical device of the present invention of a selected doubly charged ion of the enriched biomarker candidate;

FIG. 8 shows mass spectra of proteolytic cleavage products of a protein analyte, demonstrating that increased sequence coverage is obtainable by capturing proteolytic fragments on an affinity capture probe, followed by selective elution prior to analysis;

FIG. 9 shows the MALDI mass spectrum of a tryptic digest of BSA, spiked with 2M urea;

FIG. 10 shows the mass spectrum of a tryptic digest of BSA, spiked with 2M urea, after adsorption to an affinity capture probe having weak cation exchange surfaces and wash with buffer at pH 6;

FIG. 11 tabulates m/z of peptides observed in mass spectra obtained from a tryptic digest of BSA, spiked with 2M urea, after adsorption to an affinity capture probe having weak cation exchange surfaces and washed under varying conditions;

FIG. 12 tabulates m/z of peptides observed in mass spectra obtained from a tryptic digest of BSA, spiked with 2M urea, after adsorption to an affinity capture probe having strong anion exchange surfaces and washed under varying conditions;

FIG. 13 shows mass spectra at three stages of CEA capture on a ProteinChip® Array;

FIG. 14 shows mass spectra after on-chip pepsin digestion of the ProteinChip® Arrays of FIG. 13;

FIG. 15 shows the MS/MS spectrum of CEA peptide MH⁺=m/z 1894.9299 obtained using SELDI-QqTOF according to the present invention;

FIG. 16 shows mass spectra of pepsin digests of serial dilutions of CEA from 400 fmol/μl to 4 fmol/μl, normalized using somatostatin;

FIG. 17 is a plot of the intensities of the CEA-reporting peptide (m/z=1896) against the amount of CEA loaded on the chip from the spectra of FIG. 16, with linear response observed from 20 fmol to 80 fmol;

FIG. 18 shows mass spectra from a serial dilution of CEA in the presence of fetal calf serum;

FIG. 19 shows mass spectra from serial dilution of CEA in the presence of fetal calf serum after pepsin proteolysis;

FIG. 20 shows mass spectra of media samples drawn from cells grown under normal or hypoxic conditions;

FIG. 21 shows mass spectra of samples drawn from cells grown under normal or hypoxic conditions after trypsin digestion; and

FIG. 22 depicts positive-ion mass spectra of peptide products resulting from 4 hr on-chip acid hydrolysis, as analyzed by the Ciphergen Biosystems PBS II MS, with conditions as follows: (a) 6% TFA, apo-Mb; (b) 0.6% TFA, apo-Mb; (c) 6% TFA, lysozyme; and (d) 0.6% TFA, lysozyme;

FIG. 23 shows the PBSII mass spectra (protein profiles) for a sample of cytochrome C in fetal calf serum (panels A and B, with B at increased zoom) and for a control (FCS, panels C and D, with D at increased zoom);

FIG. 24 shows MS spectra for control and sample, as in FIG. 23, acquired after on-chip digestion with trypsin;

FIG. 25 shows spectra for sample and control, as in FIG. 24, but acquired on a QqTOF tandem mass spectrometer;

FIG. 26 shows the QqTOF CID MS/MS fragment spectrum for the peptide at 1168; and

FIG. 27 shows the MS-Tag results from submission of the peptide fragment masses from the spectrum shown in FIG. 26.

### DETAILED DESCRIPTION OF THE INVENTION

I. Definitions

As used herein, the terms set forth with particularity below have the following definitions. If not otherwise defined, all terms used herein have the meaning commonly understood by a person skilled in the arts to which this invention belongs.

“Analyte” refers to any component of a sample that is desired to be detected. The term can refer to a single component or a plurality of components in the sample.

“Probe” refers to a device that, when positionally engaged in interrogatable relationship to a laser desorption ionization source and in concurrent communication at atmospheric or subatmospheric pressure with a gas phase ion spectrometer, can be used to introduce ions derived from an analyte into the spectrometer. As used herein, the “probe” is typically reversibly engageable by a probe interface.

“Affinity capture probe” refers to a probe that binds analyte through an interaction that is sufficient to permit the probe to extract and concentrate the analyte from an inhomogeneous mixture. Concentration to purity is not required. The binding interaction is typically mediated by adsorption of analyte to an adsorption surface of the probe. Affinity capture probes are often colloquially referred to as “protein biochips”, which phrase is thus used herein synonymously with “affinity capture probe”. The term “ProteinChip® Array” refers to affinity capture probes that are commercially available from Ciphergen Biosystems, Inc., Fremont, Calif., for use in the present invention. Affinity capture probes can have chromatographic adsorption surfaces or biomolecule affinity surfaces, as hereinafter defined.

“Adsorption” refers to detectable noncovalent binding of an analyte to an adsorbent.

“Adsorbent” refers to any material capable of adsorbing an analyte. The term “adsorbent” is used herein to refer both to a single material (“monoplex adsorbent”) (e.g., a compound or a functional group) and to a plurality of different materials (“multiplex adsorbent”). The adsorbent materials in a multiplex adsorbent are referred to as “adsorbent species.” For example, a laser-addressable adsorption surface on a probe substrate can comprise a multiplex adsorbent characterized by many different adsorbent species (e.g., anion exchange materials, metal chelators, or antibodies) having different binding characteristics.

“Adsorption surface” refers to a surface having an adsorbent.

“Chromatographic adsorption surface” refers to a surface having an adsorbent capable of chromatographic discrimination among or separation of analytes. The phrase thus includes surfaces having ion extraction moieties, anion exchange moieties, cation exchange moieties, normal phase moieties, reverse phase moieties, metal affinity capture moieties, and/or mixed-mode adsorbents, as such terms are understood in the chromatographic arts.

“Biomolecule affinity surface” refers to a surface having an adsorbent comprising biomolecules capable of specific binding, such as proteins, oligosaccharides, antibodies, receptors, small molecular ligands, as well as various protein lipo- and glycoconjugates.

The “complexity” of a sample adsorbed to an adsorption surface of an affinity capture probe means the number of different protein species that are adsorbed.

“Specific binding” refers to the ability of two molecular species concurrently present in a heterogeneous (inhomogeneous) sample to bind to one another preferentially over binding to other molecular species in the sample. Typically, a specific binding interaction will discriminate over adventitious binding interactions in the reaction by at least two-fold, more typically more than 10- to 100-fold. When used to detect analyte, specific binding is sufficiently discriminatory when determinative of the presence of the analyte in a heterogeneous (inhomogeneous) sample. Typically, the affinity or avidity of a specific binding reaction is least about 10⁻⁷ M, with specific binding reactions of greater specificity typically having affinity or avidity of at least 10⁻⁸ M to at least about 10⁻⁹ M.

“Energy absorbing molecules” and the equivalent acronym “EAM” refer to molecules that are capable of absorbing energy from a laser desorption ionization source and thereafter contributing to the desorption and ionization of analyte in contact therewith. The phrase includes all molecules so called in U.S. Pat. Nos. 5,719,060, 5,894,063, 6,020,208, and 6,027,942, the disclosures of which are incorporated herein by reference in their entireties, includes EAM molecules used in MALDI, frequently referred to as “matrix”, and explicitly includes cinnamic acid derivatives, sinapinic acid (“SPA”), cyano hydroxy cinnamic acid (“CHCA”) and dihydroxybenzoic acid.

“Tandem mass spectrometer” refers to any gas phase ion spectrometer that is capable of performing two successive stages of m/z-based discrimination or measurement of ions, including of ions in an ion mixture. The phrase includes spectrometers having two mass analyzers that are capable of performing two successive stages of m/z-based discrimination or measurement of ions tandem-in-space. The phrase further includes spectrometers having a single mass analyzer that are capable of performing two successive stages of m/z-based discrimination or measurement of ions tandem-in-time. The phrase thus explicitly includes QqTOF mass spectrometers, ion trap mass spectrometers, ion trap-TOF mass spectrometers, TOF-TOF mass spectrometers, and Fourier transform ion cyclotron resonance mass spectrometers.

“Eluant” refers to an agent, typically a solution, that is used to affect or modify adsorption of an analyte to an adsorbent of an adsorption surface. Eluants also are referred to herein as “selectivity threshold modifiers.”

“Elution characteristic” refers to a physical or chemical characteristic of an eluant that contributes to its ability to affect or modify adsorption of an analyte to an adsorbent of an adsorption surface. Two eluants have different elution characteristics if, when put in contact with an analyte and adsorbent, the degree of affinity of the analyte for the adsorbent differs. Elution characteristics include, for example, pH, ionic strength, degree of chaotropism, detergent strength, and temperature.

“Biologic sample” and “biological sample” identically refer to a sample derived from at least a portion of an organism capable of replication. As used herein, a biologic sample can be derived from any of the known taxonomic kingdoms, including virus, prokaryote, single celled eukaryote and multicellular eukaryote. The biologic sample can derive from the entirety of the organism or a portion thereof, including from a cultured portion thereof. Biologic samples can be in any physical form appropriate to the context, including homogenate, subcellular fractionate, lysate and fluid. “Complex biologic sample” refers to a biologic sample comprising at least 100 different protein species. A “moderately complex biologic sample” refers to a biologic sample comprising at least 20 different protein species.

“Biomolecule” refers to a molecule that can be found in, but need not necessarily have been derived from, a biologic sample.

“Organic biomolecule” refers to an organic molecule that can be found in, but need not necessarily have been derived from, a biologic sample, such as steroids, amino acids, nucleotides, sugars, polypeptides, polynucleotides, complex carbohydrates and lipids, as well as combinations thereof.

“Small organic molecule” refers to organic molecules of a size comparable to those organic molecules generally used in pharmaceuticals. The term excludes organic biopolymers (e.g., proteins, nucleic acids, etc.). Small organic molecules as used herein typically range in size up to about 5000 Da, up to about 2500 Da, up to about 2000 Da, or up to about 1000 Da.

“Biopolymer” refers to a polymer that can be found in, but need not necessarily have been derived from, a biologic sample, such as polypeptides, polynucleotides, polysaccharides and polyglycerides (e.g., di- or triglycerides).

“Fragment” refers to the products of the chemical, enzymatic, or physical breakdown of an analyte. Fragments may be in a neutral or ionic state.

The terms “polypeptide”, “peptide”, and “protein” are used interchangeably herein to refer to a naturally-occurring or synthetic polymer comprising amino acid monomers (residues), where amino acid monomer here includes naturally-occurring amino acids, naturally-occurring amino acid structural variants, and synthetic non-naturally occurring analogs that are capable of participating in peptide bonds. Polypeptides can be modified, e.g., by the addition of carbohydrate residues to form glycoproteins. The terms “polypeptide,” “peptide” and “protein” include glycoproteins as well as non-glycoproteins.

“Polynucleotide” and “nucleic acid” equivalently refer to a naturally-occurring or synthetic polymer comprising nucleotide monomers (bases). Polynucleotides include naturally-occurring nucleic acids, such as deoxyribonucleic acid (“DNA”) and ribonucleic acid (“RNA”), as well as nucleic acid analogs. Nucleic acid analogs include those which include non-naturally occurring bases, and those in which nucleotide monomers are linked other than by the naturally-occurring phosphodiester bond. Nucleotide analogs include, for example and without limitation, phosphorothioates, phosphorodithioates, phosphorotriesters, phosphoramidates, boranophosphates, methylphosphonates, chiral-methyl phosphonates, 2-O-methyl ribonucleotides, peptide-nucleic acids (PNAs), and the like.

As used herein, “molecular binding partners”—and equivalently, “specific binding partners”—refer to pairs of molecules, typically pairs of biomolecules, that exhibit specific binding. Nonlimiting examples are receptor and ligand, antibody and antigen, biotin and avidin, and biotin and streptavidin.

“Receptor” refers to a molecule, typically a macromolecule, that can be found in, but need not necessarily have been derived from, a biologic sample, and that can participate in specific binding with a ligand. The term further includes fragments and derivatives that remain capable of specific ligand binding.

“Ligand” refers to any compound that can participate in specific binding with a designated receptor or antibody.

“Antibody” refers to a polypeptide substantially encoded by at least one immunoglobulin gene or fragments of at least one immunoglobulin gene, that can participate in specific binding with a ligand. The term includes naturally-occurring forms, as well as fragments and derivatives. Fragments within the scope of the term as used herein include those produced by digestion with various peptidases, such as Fab, Fab′ and F(ab)′2 fragments, those produced by chemical dissociation, by chemical cleavage, and recombinantly, so long as the fragment remains capable of specific binding to a target molecule. Typical recombinant fragments, as are produced, e.g., by phage display, include single chain Fab and scFv (“single chain variable region”) fragments. Derivatives within the scope of the term include antibodies (or fragments thereof) that have been modified in sequence, but remain capable of specific binding to a target molecule, including interspecies chimeric and humanized antibodies. As used herein, antibodies can be produced by any known technique, including harvest from cell culture of native B lymphocytes, hybridomas, recombinant expression systems, by phage display, or the like.

“Antigen” refers to a ligand that can be bound by an antibody. An antigen need not be immunogenic. The portions of the antigen that make contact with the antibody are denominated “epitopes”.

“Fluence” refers to the energy delivered per unit area of interrogated image.

II. Affinity Capture Probe Tandem Mass Spectrometer

In a first aspect, the present invention provides an analytical instrument that combines the advantages of affinity capture laser desorption ionization sample introduction with the advantages of high accuracy, high mass resolution, tandem mass spectrometers. The combination provides significant advantages over existing devices for performing known techniques. Furthermore, the new instrument makes possible new methods of protein discovery and makes possible new methods of identifying and characterizing molecular interactions between and among specific binding partners that are at once more efficient and more sensitive than existing approaches. The instrument will first briefly be described as a whole; thereafter, features of the affinity capture probe interface will be described in greater detail.

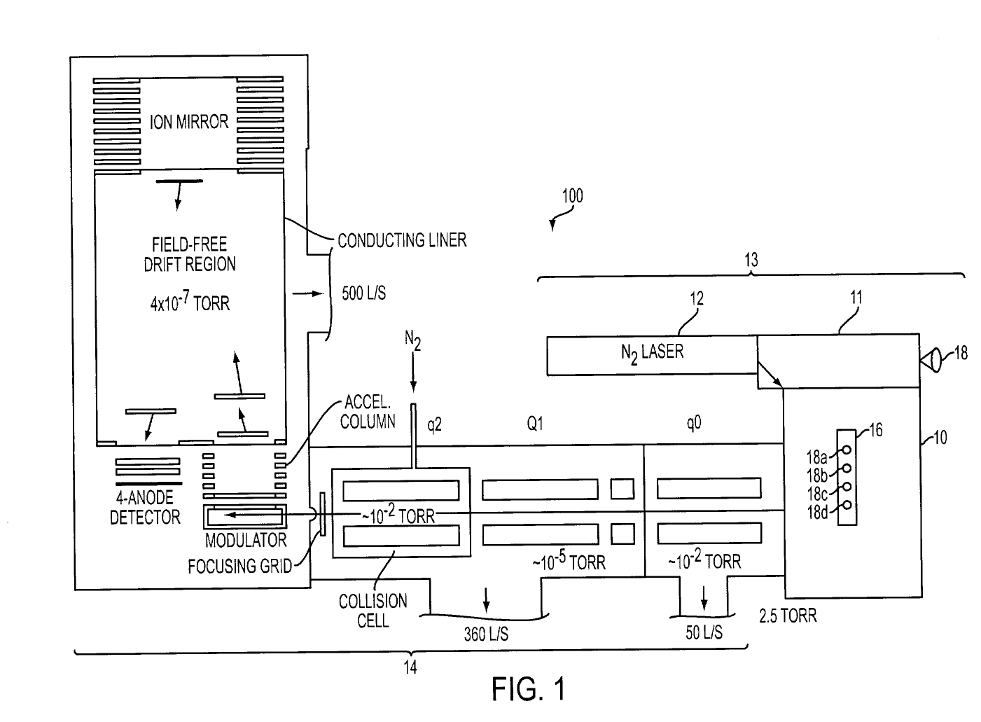

Briefly, with reference to FIG. 1, instrument 100 comprises laser desorption/ionization source 13; affinity capture probe interface 10, and tandem mass spectrometer 14. Shown in FIG. 1 is a preferred embodiment in which laser source 12 is a pulsed nitrogen laser and tandem mass spectrometer 14 is an orthogonal quadrupole time-of-flight mass spectrometer (QqTOF) tandem MS.

Laser Desorption/Ionization Source

Laser desorption/ionization source 13 produces energetic photons that, properly conditioned and directed, desorb and ionize proteins and other analytes adherent to affinity capture probe 16. Laser desorption/ionization source 13 comprises laser source 12, laser optical train 11, and, optionally, probe viewing optics 18.

Laser desorption/ionization source 13 produces pulsed laser energy either through use of a pulsed laser 12 or, alternatively, by mechanically or electronically chopping the beam from a continuous laser 12. Typically, pulsed lasers are preferred. Preferred pulsed laser sources include nitrogen lasers, Nd:YAG lasers, erbium:YAG lasers, and CO₂ lasers. Presently preferred is a pulsed nitrogen laser, due to simple footprint and relatively low cost.

Photons emitted from laser 12 are directed to strike the surface of probe 16 by laser optical train 11. Optical train 11 can consist of an arrangement of lenses, mirrors, prisms, attenuators, and/or beam splitters that function to collect, direct, focus, sub-divide, and control the intensity of each laser pulse so that an appropriate desorption fluence in the form of a focused spot of desorption energy is delivered to probe 16.

Alternatively, optical train 11 can consist of a fiber optic array that functions to collect, direct, and sub-divide the energy of each laser pulse.

In this latter embodiment, the output of laser 12 is coupled to the input side of an optical fiber using an optical coupler; the coupler is typically comprised of a lens whose focal length and diameter is appropriate for the input numerical aperture of the fiber.

The amount of energy entering the fiber can be controlled by prudent adjustment of the lens position with respect to the fiber; in this instance, the fiber optical coupler can double as an optical attenuator. In another preferred arrangement, the total output energy of the laser is coupled into the fiber and an attenuator is placed between the output side of the optical fiber and the desorption spot focusing elements of the optical train. In yet another preferred arrangement, an optical attenuator is placed between the laser and the optical fiber coupler. In all instances, optical attenuation is employed to insure the delivery of appropriate laser fluence to the surface of probe 16 independent of the output energy of laser 12. Typical laser fluences are on the order of 20-1000 μjoules/square millimeter.

As it is well established that fiber optic components can often be damaged when accepting focused energy from lasers, it is advantageous to maximize the acceptance area of the input side of the fiber so that the fluence of the incident laser energy is below the damage threshold of the fiber. The latter also simplifies alignment of the laser beam with the optical fiber when adjusting the relative position of the optical coupler with respect of the laser and optical fiber. However, in order to obtain reasonable desorption fluence levels at probe 16, a maximum exit side fiber diameter of 400 μm (microns) should not be exceeded when used with typical nitrogen lasers delivering a maximum energy of about 200 μJ/laser pulse. A solution to this problem lies in the incorporation of a tapered optical fiber whose input side has a diameter on the order of 400 to 1200 microns and the output side of which has a diameter of 200 to 400 microns.

Typically, the desorption spot should be focused to a size that maximizes the generation of ions for each pulse by interrogating the greatest area of probe 16 while maintaining sufficient fluence to induce desorption and ionization. While using typical nitrogen lasers delivering a maximum energy of about 200 μJ/pulse in a laser desorption/ionization source coupled to a quadrupole-quadrupole time-of-flight tandem mass spectrometer, an optimum laser spot area has been determined to range between 0.4 and 0.2 square millimeters.

Laser desorption/ionization source 13 can include, typically as an integral part of optical train 11, probe viewing optics 18. Viewing optics 18 can contain an illumination source, lenses, mirrors, prisms, dichroic mirrors, band-pass filters, and a CCD camera to allow the illumination and viewing of the desorption locus, i.e., the region of probe 16 to be interrogated by laser.

Where laser optical train 11 comprises an optical fiber, viewing optics 18 can take advantage of light from the optical fiber itself.

For example, the fiber optic coupler can be bifurcated to split off a small fraction of the laser excitation energy to be used as a means of monitoring the applied laser energy, or it can be bifurcated to allow the introduction of visible light to illuminate the desorption locus.

In the first of these two embodiments, a small fraction of the excitation energy is directed to impinge upon a photo-detector that is an integral component of a laser energy circuit calibrated to reflect the actual amount of laser energy delivered to probe 16. In the second embodiment, visible light is directed to illuminate the desorption locus making viewing of this region possible, either through a separate set of photo optics coupled to a CCD camera or by the employment of a prism or dichroic mirror, between the optical fiber and the laser excitation source, that directs light reflected up the main branch of the optical fiber towards a CCD camera.

Alternatively, a prism or dichroic mirror can be placed in line between the illuminating fiber branch of the optical fiber and the illumination source to allow any back reflected images that couple into this branch to be directed to impinge upon a CCD camera. In yet another embodiment, the fiber can be trifurcated so that one branch delivers desorption/ionization laser pulses, the second branch delivers visible light for illuminating the desorption locus, and the third branch transmits reflected light from the desorption locus to a CCD camera. For each of these viewing schemes, an appropriate band-pass filter should be deployed between the CCD camera and viewing optical train to prevent the transmission of possibly damaging high energy photons that arise as the direct reflection of the incident laser pulse upon the probe surface or that are secondary photons emitted from the probe surface as a direct consequence of electronic excitation by the incident laser pulse.

Probe Interface

Affinity capture probe interface 10 is capable of reversibly engaging affinity capture probe 16 and of positioning probe 16 in interrogatable relationship to laser source 12 and concurrently in communication with tandem mass spectrometer 14; the communication supports atmospheric to subatmospheric pressure.

Probe interface 10 comprises a probe holder, probe introduction port, probe position actuator assembly, vacuum and pneumatic assembly, and an interface ion collection system.

The probe holder is a component of probe interface 10 shaped to conform to the form factor of probe 16. Where probe 16 is a ProteinChip® Array (Ciphergen Biosystems, Inc., Fremont, Calif. USA), the probe holder conforms to the form factor of the ProteinChip® Array.

The probe holder can hold a single probe 16 or a plurality of probes 16. The holder positions each probe 16 in proper orientation to be interrogated by laser desorption/ionization source 13 and with respect to the interface ion collection system.

The probe holder makes intimate contact with a position actuator assembly.

The actuator assembly moves the relative position of probe 16 with respect to laser desorption/ionization source 13 and the interface ion collection system so that different regions of the probe can be interrogated and ions resulting from such irradiation collected for introduction into tandem mass spectrometer 14.

The actuator comprises electro-mechanical devices that support translational and/or rotational movement of probe 16 while maintaining the probe's position with respect to the laser desorption/ionization source and ion collection system constant. Such electromechanical devices include but are not limited to mechanical or optical position sensors, solenoids, stepper motors, DC or AC synchronous motors that either directly or indirectly communicate with linear motion actuators, linear or circular motion guide rails, gimbals, bearings, or axles.

A probe introduction port allows the probe holder, containing loaded probes 16, to be placed onto the probe position actuator assembly without introducing undue levels of atmospheric gas into the probe interface 10 and tandem mass spectrometer 14.

In order to accomplish the latter, the probe introduction port uses a vacuum evacuation system (the probe introduction port evacuation system) to pump out atmospheric gas, achieving a target port pressure prior to moving the chip into the working position. During probe exchange, the probe actuator assembly moves the probes from the working position (that position in alignment with laser desorption source 13 and the ion collection system) to an exchange position. In doing so, the actuator can provide a seal between the exchange port that is soon to be raised to atmospheric pressure, and the inlet of the mass spectrometer. After sealing off the mass spectrometer inlet, atmospheric gas is introduced into the probe introduction port by a probe introduction port pressurization system. This eliminates the pressure difference between the atmospheric surface of the probe holder and the introduction port, allowing the probe holder to be removed from the probe position actuator assembly.

Following the removal of previously analyzed probes 16 and the installation of new probes 16, the probe holder is replaced into its position actuator and the sample loading process begins. As previously described, the probe introduction port can be pumped down to sub-atmospheric pressure by the evacuation system. Upon achieving the target sample introduction pressure, the probe actuator system moves probe 16 from the exchange position to the working position, and in doing so opens the seal to the mass spectrometer inlet.

Where, alternatively, ions are generated in a desorption chamber held at atmospheric pressure and ultimately directed to an ion optic assembly that introduces the ions to the mass spectrometer inlet, it is not necessary to evacuate and pressurize the probe introduction port since it will be maintained at atmospheric pressure.

The probe introduction port evacuation system comprises a vacuum pump, pressure sensor, vacuum compatible tubing and connecting fittings, as well as vacuum compatible valves that, when acting in concert, allow the controlled evacuation of atmospheric gas contained within the introduction port following sample exchange so that probes 16 can be moved into the working position. The vacuum pump can be, but is not limited to, a single stage or multi-stage oil mechanical pump, a scroll pump, or oil-free diaphragm pump.

In a preferred embodiment, the vacuum compatible valves are electrically controlled solenoid valves. In the same embodiment, the pressure sensor is an electronic sensor capable of operating in pressure domains ranging from atmospheric pressure to 1 millitorr. Such pressure sensors include but are not limited to thermocouple gauges and pirani gauges. In the same embodiment, concerted operation of this system is achieved under logic control provided by an analog logic circuit or digital microprocessor that reconciles inputs from the pressure sensor and positional sensors to allow for automated evacuation of the sample port as part of the overall instrument operation.

The probe introduction port pressurization system comprises a gas source, pressure sensor, gas conducting tubing and fittings, and gas compatible valves that, when acting in concert, allow the controlled introduction of gas that pressurizes the exchange port, thus allowing removal of the probe holder from the actuator assembly.

In one embodiment, the gas source is untreated atmospheric gas. In another embodiment, the gas source is atmospheric gas that is first directed through a moisture absorbent trap and optionally secondly through a particulate filter prior to introduction to the pressurization system. In another embodiment, pressurizing gas is supplied by a purified source of dry inert gas such as nitrogen or any of the cost-effective noble gases in lieu of using atmospheric gas.

In a preferred embodiment, the gas conducting tubing, fittings, some of the valves, and pressure sensor of the pressurization system are those used in the evacuation system. In the same embodiment, concerted operation of this system is achieved under logic control provided by an analog logic circuit or digital microprocessor that utilizes inputs from the pressure and positional sensors to allow for automated pressurizing of the sample port as part of the overall instrument operation.

The probe interface pressure regulation system functions to provide selective background gas pressure in the desorption chamber that exists between the sample presenting (adsorption) surface of probe 16 and the ion collection system. Acceptable desorption chamber pressure ranges extend from atmospheric pressure to 0.1 microtorr. A preferred pressure range extends from 1 torr to 1 millitorr. The probe interface pressure regulation system comprises a gas source, gas conducting tubing and fittings, a gas flow regulator, and a pressure sensor. The gas source can be untreated atmospheric gas. In another embodiment, the gas source is atmospheric gas that is first directed through a moisture absorbent trap and optionally secondly through a particulate filter prior to introduction to the regulation system. In another embodiment, regulation gas is supplied by a purified source of dry inert gas such as nitrogen or any of the cost-effective noble gases. The gas flow regulator may be a manually controlled flow restrictor.

Alternatively, gas flow regulation may be achieved by using an electronically controlled flow restrictor. In a preferred embodiment, close loop control of preferred desorption chamber pressure is achieved in an automated fashion under logic control provided by an analog logic circuit or digital microprocessor that actively interacts with an automated gas flow regulator to achieve a pre-established reading from the pressure gauge.

The interface ion collection system comprises an electrostatic ion collection assembly, an optional pneumatic ion collection assembly, and an electrostatic or RF ion guide.

The electrostatic ion collection assembly comprises an arrangement of DC electrostatic lens elements that function to collect ions desorbed within the desorption chamber and direct them towards the mass spectrometer inlet.

In one embodiment, the electrostatic ion assembly comprises two electrostatic elements. The first element is comprised of the probe holder and probe surface and the second is an extractor lens. The extractor lens is arranged to be between 0.2 to 4 mm away from the surface of the probe. The extractor lens contains an aperture ranging from 2 mm to 20 mm in diameter that is concentrically located about a normal axis that extends from the center of the desorption locus to the center of the mass spectrometer inlet. Independent DC potentials are applied to each element of this assembly.

In a preferred embodiment, the extractor lens contains a 10 mm diameter aperture and is located 1 mm away from the probe surface. In the same preferred embodiment, a ten volt potential difference is established between the extractor and array.

The optional pneumatic ion collection assembly comprises a gas source, conducting tubing, tubing connectors, gas flow regulators, gas pressure sensors, and a gas emission port so that a predetermined flow of gas can be created to assist the bulk transfer of desorbed ions within the desorption chamber into the mass spectrometer inlet.

The gas source can be untreated atmospheric gas. In another embodiment, the gas source is atmospheric gas that is first directed through a moisture absorbent trap and optionally secondly through a particulate filter prior to introduction to the system. In another embodiment, ion collection gas is supplied by a purified source of dry inert gas such as nitrogen or any of the cost-effective noble gases.

The gas flow regulator can be a manually controlled flow restrictor. Alternatively, gas flow regulation can be achieved by using an electronically controlled flow restrictor. The pressure sensor(s) can be but is not limited to thermocouple gauges and pirani gauges. The gas emission port is located behind probe 16 to induce bulk gas flow around the probes and down the normal axis centrally located between the desorption locus and the mass spectrometer inlet.

In a preferred embodiment, the flow of gas is under automatic closed loop control by the use of analog or digital control circuitry so that an adequate ion-sweeping flow is generated without over-pressurizing the desorption chamber.

The final component of the interface ion collection system is the ion guide. The ion guide functions to transfer the collected ions into mass spectrometer 14. It can be of the electrostatic or RF variety. A preferred embodiment is a multipolar RF ion guide. An example of the latter is a quadrupole or hexapole ion guide. In the preferred Qq-TOF instrument described in greater detail below, the ion guide is a quadrupole RF ion guide. Ions are directed into the ion guide by electrostatic and pneumatic accelerative forces, respectively created by the electrostatic and pneumatic ion collection systems. In a preferred embodiment the DC electrostatic potential of the ion guide is less than that of the extractor lens by typically 10 to 20 volts.

Tandem Mass Spectrometer

The analytical instrument of the present invention further includes tandem mass spectrometer 14. Tandem mass spectrometer 14 can usefully be selected from the group that includes orthogonal quadrupole time-of-flight (Qq-TOF), ion trap (IT), ion trap time-of-flight (IT-TOF), time-of-flight time-of-flight (TOF-TOF), and ion cyclotron resonance (ICR) varieties.

Presently preferred, and further described in detail below, is an orthogonal Qq-TOF MS.

The major strengths of the QqTOF MS are outstanding mass accuracy and resolving power; enhanced sensitivity in the peptide and low mw range; and superior ms/ms performance by employing low energy collision induced dissociation (CID). An orthogonal QqTOF with electrospray ionization source is available commercially from AB/MDS Sciex (QSTAR™; AB/MDS-Sciex, Foster City, Calif., USA).

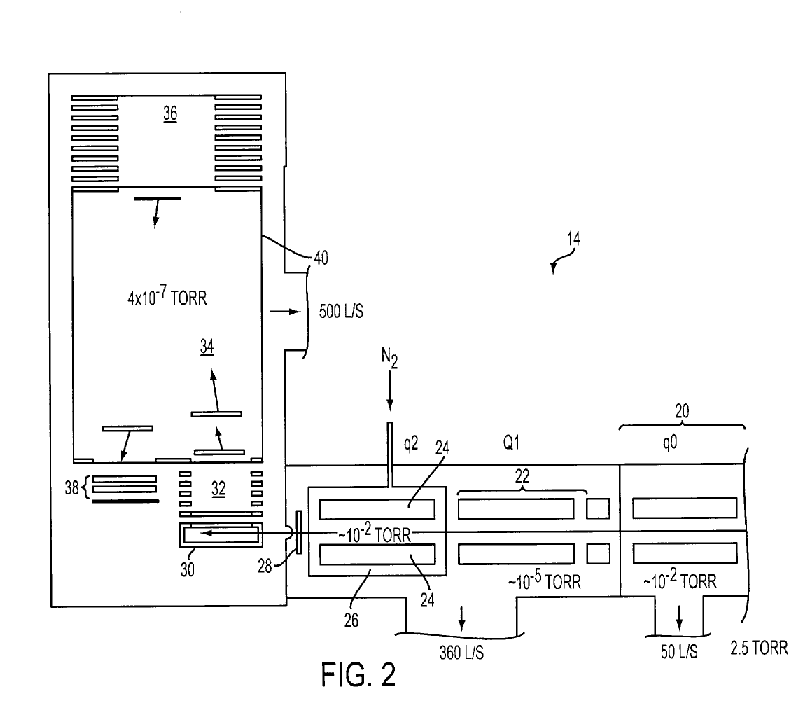

With reference to FIG. 2, the principles and features of the QqTOF will be briefly outlined.

Ions are created in a desorption chamber prior to the first quadrupole lens “q0”. Pressure within q0 is typically maintained at about 0.01 to 1 torr, but can also be maintained at atmospheric pressure. In this manner, desorbed ions are rapidly cooled by collisions with the background gas shortly after their formation.

This cooling or damping of the ion population provides three major advantages.

First, the cooling eliminates the initial energy distributions of the desorbed ions and reduces their total energy down to a point that approximates their thermal energy. This simplifies the orthogonal extraction requirement, compensating for variations in ion position and energy, thus improving ultimate resolving power. A direct consequence of this improved resolution is enhanced mass accuracy down to the low ppm level.

The second major advantage of collisional cooling is its ability to decrease the rate of long term ion decay. Gas collisions relax internal excitation and improve the stability of peptide and protein ions. This stabilizing effect appears to be maximized when ions are created in the presence of about 1 torr pressure of background gas. Measurements published by others have indicated that losses of small groups and background fragmentation can be practically eliminated, improving the transmission of high mw proteins and other labile biopolymers (i.e.glyco-conjugates, DNA, etc.). Faster decay mechanisms (prompt and in-source type decay) still occur.

The final advantage of q0 collisional cooling is in the creation of a pseudo-continuous flow of ions into the mass analyzer. Ion collisions in q0 cause the desorption cloud to spread out along the axis of q0. This spreading creates a situation in which ions from various desorption events begin to overlap, creating an electrospray-like continuous introduction of ions into the analyzer.

After passing through q0, ions enter a second quadrupole 22 (“Q1”). This quadrupole functions as either an ion guide or as a mass filter. It is here that ion selection is created for ms/ms or single ion monitoring (SIM) experiments.

After exiting Q1, ions enter a third quadrupole 24 (“q2”) positioned in collision cell 26. During simple experiments, q2 is operated as a simple rf ion guide. For ms/ms experiments, q2 is filled with collision gas at a pressure of about 10⁻² torr to promote low energy CID.

After exiting q2, ions are slightly accelerated by a DC potential difference applied between the exit of q2 and focusing grid 28. This acceleration “biases” the velocities of the ions in the Y-axis so that their velocities are now inversely related to the square root of their m/z. This must be accomplished if all ions of different m/z are to strike the detector after orthogonal extraction and free flight. If such biasing is not accomplished, ions of different m/z will enter the orthogonal extraction region with the same Y-axis velocity.

As always in time-of-flight, ions of lower m/z will strike the detector before ions of greater m/z. The absolute degree of displacement in the Y-axis will be a product of an ion's flight time in the Z-axis and an ion's Y-axis velocity. If the detector is placed at some location optimized for intermediate mw ions, lighter ions will “undershoot” the detector arriving to the right side of the detector in FIG. 2. Conversely, ions of greater m/z will “overshoot” the detector and arrive at the left side of the detector in FIG. 2. Consequently, it is necessary for all ions to maintain a constant ratio of Z- and Y-axis velocities if all ions are to strike a common detection point. The previously described grid biasing method accomplishes this.

After passing through focusing grid 28, ions arrive in modulator region 30 of the orthogonal extraction elements. Modulator 30 is pulsed at rates approaching 10,000 pulses/second (10 kHz). Ions are pushed into accelerator column 32 of the ion optic and exit out into free flight region 34 of the orthogonal time-of-flight (0-TOF). Energy correction is achieved when the ions enter ion mirror 36. In the mirror, ions are turned around and are directed to strike fast response, chevron array microchannel plate detector 38.

Alternatives to this prototypical arrangement can be used.

For example, the geometry presented above presents the difficulty of performing O-TOF at high acceleration energies. It is well established that ion detection sensitivity for peptides and proteins is improved as total ion energy increases. For human insulin (MW=5807.65 Da), detection efficiency approaches 100% at ion energies of 35 keV when using typical microchannel plate detectors. If the ions are to be accelerated to 20 or 30 keV of energy, free flight tube liner 40 and other corresponding components must be floated to −20 kV or −30 kV, respectively. The difficulties in providing stable electrical isolation on simple ion optic elements at such potentials are well known. To safely and reliably float a plurality of elements at such potentials is difficult. One solution is the employment of post-acceleration technology.

Unlike the device described above, such an alternative device employs a detector post accelerator (not shown). Ions are accelerated to about 4 keV of energy after leaving the orthogonal extraction elements and the free flight region is floated at −4 kV. Further acceleration is achieved as ions enter a post-accelerator detector assembly. In this assembly, ions pass through a field-retaining grid held at liner potential. Ions then receive additional acceleration in a field established between the field-retaining grid and the primary ion conversion surface of the detector. Such acceleration fields are on the order of 10 to 20 kV over 4 to 10 mm distances.

Because the orthogonal design uncouples the time of flight measurement from ion formation, a number of advantages are realized.

Laser fluence related problems, such as peak broadening due to ion shielding and ion acceleration field collapse, are eliminated because ions of the desorption plume have an extended period of time (typically a few milliseconds) to expand and cool prior to orthogonal extraction and acceleration into the TOF mass analyzer. Additionally, orthogonal extraction eliminates much of the large hump and baseline anomaly seen at the beginning of high laser energy, conventional extraction spectra due to the chemical noise created by the excessive neutral load of the EAM. Because neutrals are not extracted in the modulator region, only ions are transmitted down to the detector and chemical noise is appreciably reduced.

These factors allow the use of laser fluences that are 2-3 times greater than those normally employed during parallel continuous or delayed ion extraction approaches. The net result is an almost complete elimination of the need to hunt and search for “sweet spots” even in the presence of poor sample-EAM homogeneity, as well as improved external standard mass accuracy determination (typical errors are between 20-50 ppm), improved quantitative reproducibility, and improved signal to noise. An additional benefit is the elimination of the need to perform low and high laser energy scans to analyze ions of a broad m/z range. A single laser fluence can now be employed to see both low and high mw ions, greatly simplifying the analysis of unknown mixtures.

Perhaps one of the most impressive advantages of this device when compared to conventional parallel extraction approaches lies in its ability to obviate the need for rigid sample positioning requirements. Because the TOF measurement is substantially removed from the ion formation process, the original position of the ion is no longer important. Furthermore, since ion formation is accomplished in a high-pressure environment without concomitant application of high voltage extraction fields, the design requirements of solid-state sample inlet systems are greatly relieved. Simple approaches can be taken to employ 2-dimensional sample manipulators while maintaining excellent, external-standard mass accuracy performance. Additionally, sample presenting surfaces no longer need to be made of metals or other conductive media.

To summarize, the laser desorption ionization (LDI) Qq-TOF MS has the following advantages over existing LDI-TOF MS technology: (1) increased external standard mass accuracy (20-50 ppm typical); (2) enhanced resolution; (3) improved ms/ms efficiency; (4) improved ease of signal production using a single high laser energy level that eliminates the need for high and low energy scans; (5) improved quantitative ability through the use of TDC technology and laser fluences 2-4 times above minimum desorption threshold; (6) reduced requirements for 2-dimension sample actuators; (7) potential for using plastic components for sample presenting probe surfaces (injection molded two dimensional probe arrays, for example); (8) reduced chemical noise by using single ion monitoring and enhanced ability to measure for ions in the EAM chemical noise domain.

The laser desorption ionization (LDI) Qq-TOF MS has the following advantages over existing MALDI-PSD approaches in protein characterization and identification.

The LDI-QqTOF provides higher mass resolving power and mass accuracy; in database mining approaches, this increased capability reduces the number of false positive database hits, simplifying identification. Furthermore, the QqTOF also provides greater than an order of magnitude greater sensitivity than can be obtained with PSD MS/MS.

The analytical instrument of the present invention demonstrates impressive MS/MS capability and less than 20 ppm mass assignment error for single MS analysis. The latter has allowed the identification of a number of proteins simultaneously retained on the surface of a single affinity capture probe.

Other Components

Affinity capture probe tandem MS instrument 100 typically further comprises a digital computer interfaced with the tandem mass spectrometer detector. The digital computer is typically further interfaced with laser desorption source 12, permitting the computer both to control ion generation and to participate in data acquisition and analysis.

Analysis software can be local to the computer or can be remote, but communicably accessible to the computer. For example, the computer can have a connection to the internet permitting use of analytical packages such as Protein Prospector, PROWL, or the Mascot Search Engine, which are available on the world wide web. The analysis software can also be remotely resident on a LAN or WAN server.

Affinity Capture Probes

To conduct analyses, such as those described in detail in sections herein below, at least one affinity capture probe 16 having adsorbed analyte is engaged in probe interface 10 in position to be interrogated by laser desorption/ionization source 13 and to deliver desorbed ions into tandem mass spectrometer 14.

Probes 16 typically have one or more adsorption surfaces 18, which surfaces can differ from one another (18a, 18b, 18c, 18d). Typically, if there are a plurality of adsorption surfaces 18, all are exposed on a common face of probe 16. When a plurality of adsorption surfaces 18 are present on a single probe surface, the probe is typically denominated a probe array; commercial embodiments available from Ciphergen Biosystems, Inc. (Fremont, Calif., USA), are denominated ProteinChip® Arrays.

Adsorption surfaces 18 are typically either chromatographic adsorption surfaces or biomolecule affinity surfaces.

Chromatographic affinity surfaces have an adsorbent capable of chromatographic discrimination among or separation of analytes. Such surfaces can thus include anion exchange moieties, cation exchange moieties, reverse phase moieties, metal affinity capture moieties, and mixed-mode adsorbents, as such terms are understood in the chromatographic arts. Biomolecule affinity surfaces have an adsorbent comprising biomolecules capable of specific binding. Such surfaces can thus include antibodies, receptors, nucleic acids, lectins, enzymes, biotin, avidin, streptavidin, Staph protein A and Staph protein G. Adsorbent surfaces are further described in a section below.

Interface 10 positions probe 16 in interrogatable relationship to laser desorption/ionization source 13. Typically, it is desired that the laser interrogate probe adsorption surfaces 18. Accordingly, interface 10 positions probe 16 adsorption surfaces 18 in interrogatable relationship to laser desorption/ionization source 13. If adsorption surfaces 18 are positioned on only one face of probe 16, probe 16 and/or the probe holder of interface 10 can be asymmetrically dimensioned, thus obligating insertion of probe 16 in the orientation that presents adsorption surfaces 18 to laser desorption source 13.

Where probe 16 has a plurality of adsorption surfaces 18, it will be desired that laser source 12 be able discretely to address each adsorption surface 18. This can be accomplished by optics interposed between laser source 12 and interface 10, by rendering laser source 12 and/or interface 10 movable, or by a combination thereof.

Probe 16 can be an affinity capture probe as is presently used in single MS analysis (e.g., ProteinChip® Arrays commercially available from Ciphergen Biosystems, Inc., Fremont, Calif. USA).

III. Applications of the Affinity Capture Probe Tandem MS Instrument

The above-described analytical instrument of the present invention provides significant advantages in, and affords novel methods for, (A) protein discovery and identification; (B) characterization of interactions between specific binding pairs; (C) sequencing and identifying proteins by tandem mass spectrometry; (D) proteolytic amplification for identification and detection (“PAID”); and (E) differential protein display and quick protein identification (“QPID”).

Advantages conferred by the analytical instrument of the present invention that are common to all five of these applications include: the ability to do high mass accuracy measurements in single mass MS and tandem MS mode, combined with affinity capture probe technology. Specific advantages will be described with respect to each application, which will now be described in turn.

A. Protein Discovery and Identification

1. Advantages of the Methods of the Invention

One related set of problems that protein biologists attempt to solve is protein discovery, identification, and assay development.

Protein discovery is the process of finding proteins in a system that are biologically interesting because, for example, they function as diagnostic markers or carry out critical cell functions. Protein identification is the process of determining the identify of a discovered protein. Assay development is the process of developing a reliable assay to detect the protein. The methods of this invention provide advantages for the practitioner in carrying out all three of these processes as compared to previous technologies.

A primary advantage of this invention is that it provides a single platform on which to carry out process steps from protein discovery to protein identification to assay development. The provision of a single platform based on surface-enhanced laser desorption ionization technology significantly decreases the time between discovery and assay validation: what used to takes months using previous technologies can now take weeks or days.

The methods of this invention also significantly reduce the amount of sample required to perform the experiments. Whereas previous methods required micromoles of analyte, the present methods can perform the same experiments with picomoles of analyte. This overcomes a significant hurdle when sample is scarce or scale-up is difficult.

Previously, protein discovery and isolation was typically accomplished using 2D electrophoretic separations, with detection by staining or Western Blots. However, comparison of gels to each other to detect differentially expressed proteins is a difficult procedure.

The discovered protein might now be identified using mass spectrometry methods. Important proteins could be isolated and ultimately fragmented in the gel with proteases and the peptide fragments could be analyzed by a mass spectrometer and appropriate bioinformatics methods. However, gels are not compatible with present mass spectrometry methods, and peptide fragments have to be removed from the gel. Because the latter process inevitably resulted sample loss, this approach required large quantities of starting protein and material. When the protein was rare, as important proteins can be, this increased the difficulty of the process.

Once identified, the practitioner needs to develop a reliable assay to detect the protein. Typically, this involves developing an ELISA assay. This technology, in turn, required the production of antibodies. This can be a time consuming task, especially if the protein of interest if difficult to produce in quantity for immunization.

Thus, prior techniques could have required three different technologies to accomplish protein discovery, protein identification and protein assay. The methods of the present invention can accomplish this with one technology.

2. Methods of Protein Discovery, Identification and Assay Development

The methods of this invention for protein discovery, identification and assay development involve (i) preparing a difference map to discover a protein or proteins of interest, (ii) identifying the protein by affinity capture probe tandem mass spectrometry, and (iii) validating using an affinity capture probe laser desorption ionization chromatographic surface assay or affinity capture probe laser desorption ionization biospecific surface assay.

The process can proceed as follows.

A protein of interest is provided or is discovered by, for example, using difference mapping of retentate studies. These methods are described in, e.g., WO 98/59362 (Hutchens and Yip), the disclosure of which is incorporated herein by reference in its entirety. Briefly, two biological samples that differ in some important respect (e.g., normal v. diseased; functional v. non-functional) are examined by retentate chromatography methods. The methods involve exposing the samples to a plurality of different chromatographic affinity and wash conditions, followed by examination of the “retained proteins” by affinity capture probe laser desorption ionization. Proteins that are differentially expressed between the two samples are candidates for further examination. Because they have been examined on a mass spectrometer, the molecular weights of these candidate proteins are known.

Normally, scores of proteins in addition to the proteins of interest will be retained on the chip. Therefore, a next optional step is to refine the affinity and wash conditions under which the protein or proteins of interest are retained so as to simplify the sample for further analysis. (These optional steps are also described in the Hutchens and Yip international patent application.) While capture of the single protein of interest is ideal, capture of no more than about ten detectable proteins is favorable. The refined method provides an improved chromatographic assay for the protein of interest.

The retained proteins are then subject to fragmentation on the probe using a proteolytic agent of choice, producing a pool of peptides (cleavage products) for subsequent study. In some cases, digestion using specific endoproteases such as trypsin may be advantageous because the cleavage pattern is known and is directly compatible with bioinformatics methods involving in silico cleavage of proteins the sequences of which have been stored in a data base and searched against using single ms spectra of experimental runs. In many other cases, digestion of adsorbed proteins is best accomplished using more aggressive proteolytic means such as highly efficient proteases that cleave at multiple locations and operate under denaturing conditions or chemical proteolytic approaches that concomitantly operate under denaturing conditions. In the latter case, the diminished degree of cleavage specificity often creates the need to perform protein identification by utilizing high resolution, high accuracy MS-MS analysis (e.g., having a mass assignment error of less than 20 parts per million and resolving power of approximately 10,000). Furthermore, the digest performed can be a limited digest, i.e., a digest that produces an average of no more than 5 protein fragments, more preferably no more than 2 protein fragments, per protein in the sample.

At this point, it may not be clear whether a particular peptide fragment is a cleavage product of the protein analyte of interest or of one of the other retained proteins. Nevertheless, the analysis proceeds by selecting one of the peptide fragments (cleavage products) (possibly at random, possibly based on information that it corresponds to the protein of interest) and subjecting the peptide to gas phase fragmentation. One such method is collision-induced dissociation (CID). The peptide need not be isolated from the chip, because the MS-MS device isolates the peptide of interest from the other peptides in the mass spectrometer. This will generate a further fragmentation pattern of the selected peptide fragment.

Using methods already established in the art, such as database mining protocols, information from the fragmentation pattern is used to interrogate a protein sequence database to generate one or more putative identity candidates for the protein from which the peptide fragment is derived.

In one approach used by such art-established protocols, a closeness-of-fit analysis is performed that measures how well the actual mass spectrum of the selected fragment matches mass spectra predicted from sequences of proteins prior-accessioned into the sequence database. Such predicted spectra are either generated during comparison or are prior-calculated and stored in a derivative database of predicted mass spectra. Proteins in the database can then be ranked based on the closeness of fit to the empiric fragment mass spectrum. Knowledge of the mass of the parent protein and the species of origin, both of which are already known, will assist in limiting the number of identity candidates generated.

An alternative approach used by such art-established protocols uses differences among fragment ion masses present within the measured fragment ion spectrum to determine at least a portion of the amino acid sequence of the selected fragment; this partial sequence is then used to query protein sequence databases, typically with additional identifying criteria, such as the mass of the unfragmented parent peptide ion, species of origin, and, if known, the mass of the protein analyte prior to proteolytic cleavage. Protein identity candidates are identified based upon the closeness-of-fit calculated between the predicted sequence and sequences prior-accessioned into a sequence database. Such query algorithms, such as BLAST (basic local alignment search tool) are known in the art and are publicly available.

The two art-established approaches to identifying a protein identity candidate are not mutually exclusive and can be performed in parallel or sequentially.

Then, the putative identity of the protein from which the peptide fragment was generated is verified. Using knowledge from the database of the primary sequence of the putative identity candidate and the cleavage pattern of the proteolytic agent used, one can predict the peptide fragments and, in particular, their molecular weights, that should be generated from the cleavage of the identity candidate by the proteolytic agent. This predicted set of fragments is then compared with the actual set of fragments generated after proteolytic cleavage of the proteins retained on the chip based on their masses. If the predicted fragments are accounted for, then one is confident that the putative identity candidate actually corresponds to the identity of one of the proteins retained on the chip. If not, then one must test other putative identity candidates through a process of elimination until the protein from which the fragment is generated is identified. At this point, the generated fragments that correspond to the identified protein can be eliminated from the total set of fragments generated as having been accounted for.

If only one protein was retained after refining the affinity and wash conditions, then all the peptide fragments will have been accounted for and the process is complete. However, if more than one protein has been retained, the situation may be more complicated. For example, the fragment used in the analysis may have been generated from the protein of interest, or it may have been generated by a protein that was retained on the chip, but that is not the protein of interest.

When more than one protein has been retained on the affinity capture probe, it is useful to repeat the steps of analyzing the peptide fragments not accounted for by the MS-MS methods described until the protein of interest is identified or all the retained proteins have been identified.

Alternatively, or in addition, the complexity of the mixture of protein cleavage products adsorbed to the affinity capture probe can be reduced before tandem MS analysis. This can usefully be accomplished by washing the probe at least once with a first eluant for a time and under conditions sufficient to increase the relative concentration among protein cleavage products adsorbed to the probe of at least one cleavage product of the protein analyte of interest. Optionally, further washes, the further washes using at least a second eluant differing from the first eluant in at least one elution characteristic, can be performed for a time and under conditions sufficient further to increase the relative concentration among protein cleavage products adsorbed to the probe of at least one cleavage product of the protein analyte of interest.

The wash can be performed directly after proteolytic cleavage and before analysis, or, alternatively or in addition, can be performed after a first MS/MS analysis by removing the probe from the analytical device of the present invention and then performing the wash before reinserting the probe for a subsequent analysis.

Finally, the protein of interest can be assayed by affinity capture probe laser desorption ionization methods using either a chromatographic surface already determined to retain the protein or a biospecific surface that can be developed for use in an affinity capture probe laser desorption ionization assay. Creation of biospecific surfaces involves providing a binding partner for the identified protein, such as an antibody, or a receptor if a receptor is known, and attaching this to the chip surface. Then, the protein of interest can be assayed by surface-enhanced laser desorption ionization mass spectrometry as already described.

B. Characterization of Molecular Interactions

The analytical instrument of the present invention makes possible, for the first time, a sensitive, efficient, single-platform approach to the study of interactions between specific binding partners.

Specific binding partner interactions are at the core of a wide spectrum of biological processes. Accordingly, the ability to measure and to characterize such interactions is a necessary prerequisite to a full understanding such processes; at the clinical level, the ability to measure and to characterize such interactions is important to an understanding of pathologic aberrations in those processes and to the rational design of agents that can be used to modulate, or even abrogate, such interactions.

For example, at the level of organized eukaryotic tissues, intercellular signaling in the mammalian nervous system is mediated through interactions of neurotransmitters with their cognate receptors. An understanding of the molecular nature of such binding interactions is necessary for a full understanding of such signaling mechanisms. At the clinical level, an understanding of the molecular nature of such binding interactions is required for a full understanding of the mechanism of signaling pathologies, and for the rational design of agents that palliate such signaling pathologies, agents useful for treatment of diseases ranging from Parkinson's disease to schizophrenia, from obsessive compulsive disorder to epilepsy.

As another example, at the circulatory level, interaction of B cell receptors with circulating antigen is required to trigger B cell clonal expansion, differentiation, and antigen-specific humoral immune response. An understanding of the antigenic epitopes that contribute to antigen recognition is critical to a full understanding of immune responsiveness. At the clinical level, such understanding is important to the design of vaccines that confer more robust humoral immunity. Analogously, interaction of T cell receptors with peptide displayed in association with MHC on antigen-presenting cells is critical to the triggering of cellular immunity. An understanding of the T cell epitopes that contribute to antigen recognition is important to the design of vaccines that confer more robust cellular immunity.

At the level of individual cells, phenotypic response to extracellular signals is mediated by at least one, most often a cascade, of intermolecular interactions, from the initial interaction of a cell surface receptor with ligand, to intracytoplasmic interactions that transduce the signal to the nucleus, to interaction of protein transcription factors with DNA, the altered patterns of gene expression leading in turn to the observed phenotypic response. For example, discriminative binding of estrogen and progesterone by ovarian cells is required for ovulation. An understanding of the molecular nature of binding interactions between steroid hormone receptors and the hormone ligand, on the one hand, and liganded receptor with steroid hormone response elements in the genome, on the other, is important for an understanding of the hormonal response. Such understanding, in turn, is important for an understanding of infertility, and for the rational design of agents—such as RU486—that are intended to abrogate ovulation, implantation, and/or fetal viability.

Such interactions are found not only in eukaryotic systems, but in prokaryotic systems and in the interaction of prokaryotes with eukaryotes. For example, certain gram negative bacteria elaborate a pilus that is required for invasion of the eukaryotic urethra; an understanding of such interaction is important to full comprehension of the pathologic process, and for the rational design of agents that can prevent such invasion.

A number of techniques are used in the art to study and map such intermolecular interactions between specific binding partners. Each has significant disadvantages.

In a first such method, one member of a specific binding pair is immobilized on an adsorbent which is packed in chromatographic column. To map the sites within the structure of the second (free) binding partner that make contact with the first (bound) binding partner, the second (free) partner is cleaved. Typically, such cleavage is by specific proteolytic enzyme, although specific chemical cleavage (e.g., by CNBr) or even nonspecific chemical hydrolysis can be done. Thereafter, the digest is passed over the column to bind those portions of the second (free) partner that still bind to the first (immobilized) partner.

The peptides of the second partner are then eluted, typically using a salt or pH gradient, and identified, typically by introducing the peptides into a mass spectrometer by MALDI or electrospray ionization.

This approach has several well known, and significant, problems. First, a large quantity of purified first binding partner is required in order to create the specific adsorbent. Second, a large quantity of second binding partner, typically purified, is required for digestion, adsorption, and elution, since each of these stages is attended by dilution effects and analyte loss. Furthermore, although the subsequent mass spectrometric analysis can be highly sensitive, interfacing the fluid phase analysis to the mass spectrometer can also occasion analyte loss.

Perhaps a more fundamental disadvantage is that, by cleaving the second binding partner before binding to the first partner, only those molecular structures on the second binding partner that are properly maintained in the peptide fragments will bind, and thereafter be detected. If, for example, an antibody binds antigen at discontinuous, rather than linear, epitopes, such discontinuous epitopes can be destroyed by fragmentation; unable to support binding to the immobilized antibody, such antigenic epitopes cannot be detected.

A second typical approach in the art is to use point mutations to map, within a protein binding partner, those residues that contribute to intermolecular binding.

This latter approach requires that the protein binding partner be cloned, desired point mutations introduced, the altered protein expressed recombinant; and the altered recombinant protein purified. Thereafter, the kinetics of binding of the altered protein to its partner are measured to determine the effect of the mutated residue(s) on the intermolecular interaction.

Less often used, the nature of the contacts between binding partners can be elucidated by X-ray crystallography of the bound partners. This technique is highly effective, and provides atomic level resolution, but requires that each binding partner be highly purified, and further requires that suitable co-crystals be formed.

The affinity capture tandem mass spectrometry instrument of the present invention provides an improved approach that requires far less starting material, obviates point mutational analysis, obviates crystallization, and substantially reduces the purity requirement.

The first step is to immobilize one of the binding partners on an affinity capture probe.

Either partner can be immobilized; it is the free partner, however, for which structural information about the binding contacts will be obtained. Using receptor/ligand interactions as exemplary of the approach, immobilizing the ligand on the probe will permit the identification of regions of the receptor that participate in binding the ligand; conversely, immobilizing the receptor on the probe will permit the identification of regions of the ligand that participate in its binding to the receptor. Where the ligand is a protein—for example a protein hormone, cytokine, or chemokine—separate experiments, using each partner in turn, will yield a bilateral understanding of the intermolecular contacts.

The probe-bound partner can be immobilized using covalent or strong noncovalent interactions. The choice will depend upon the availability of suitable reactive groups on the partner to be immobilized and on the chemical nature of the surface of the probe. Appropriate chemistries are well known in the analytical arts.

For example, where the binding partner to be immobilized has free amino groups, covalent bonds can be formed between the free amino groups of the binding partner and a carbonyldiimidazole moiety of the probe surface. Analogously, free amino or thiol groups of the binding partner can be used covalently to bind the partner to a probe surface having epoxy groups. Strong coordinate or dative bonds can be formed between free sulfhydryl groups of the binding partner and gold or platinum on the probe surface.

Optionally, remaining reactive sites on the probe surface can then be blocked to reduce nonspecific binding to the activated probe surface.

The second (free) binding partner is then contacted to the affinity capture chip and allowed to bind to the first (immobilized) binding partner.

The second (free) binding partner can be present pure in solution, if known and available, or, more typically, will be captured from a heterogeneous mixture, such as a biological sample suspected to contain the second binding partner. The biological sample, as in biomarker discovery approaches described earlier, can be a biological fluid—such as blood, sera, plasma, lymph, interstitial fluid, urine, or exudates—can be a cell lysate, a cellular secretion, or can be a partially fractionated and purified portion thereof.

The probe is then washed with one or more eluants having defined elution characteristics. These washes serve to reduce the number of species that bind nonspecifically to the probe.

Energy absorbing molecules are then applied, typically in the liquid phase, and allowed to dry. Application of energy absorbing molecules is effected in the same manner as for existing uses of affinity capture probes; where ProteinChip® Arrays (Ciphergen Biosystems, Inc., Fremont, Calif., USA) are used, energy absorbing molecules are applied according to manufacturer instructions.

Species that are noncovalently bound to the affinity capture probe—e.g., second binding partners specifically bound to the first (immobilized) binding partners, molecules nonspecifically bound to the probe surface, molecules nonspecifically bound to the first binding partners—are then detected in a first phase of laser desorption ionization mass spectrometry.

The mass spectrometer can be a single stage affinity capture LDI-MS device, such as the PBS II from Ciphergen Biosystems, Inc. (Fremont, Calif. USA). However, the affinity capture tandem MS of the present invention provides higher mass accuracy and higher mass resolution and is preferred.

Typically, the second (free) binding partner will be known from earlier studies, and its presence or absence readily confirmable by mass spectrometry. If the second (free) binding partner is unknown, each of the species bound to the probe can be investigated in turn. If the number of detectable species is too high, the affinity capture probe can be washed with eluants having different elution characteristics (typically, increased stringency), to reduce the number of species present for analysis.

Once binding of the second (“free”) binding partner to the first (immobilized) binding partner is confirmed, the second binding partner is fragmented. This is typically accomplished by contacting the second binding partner (which is, at this point, noncovalently but specifically bound to the first binding partner, which is, in turn, immobilized on the probe surface) with specific endoproteases, such as trypsin, Glu-C (V8) protease, endoproteinase Arg-C (either the serine protease or cysteine protease Arg-C enzyme), Asn-N protease, or Lys-C protease.

After digestion, peptides are detected by mass spectrometry.

If all fragments of the second binding partner are to be identified—e.g., to confirm the identity of the second binding partner by peptide mass fingerprint analysis—energy absorbing molecules can be applied and the probe used to introduce the peptides into a mass spectrometry by laser desorption ionization. For this purpose, the Ciphergen PBS II single acceleration stage linear TOF MS can be used; the tandem MS of the present invention, which provides superior mass accuracy and mass resolution is preferred, since the increased resolution and accuracy reduces the number of putative “hits” returned at any given confidence level in any given database query.

More typically, however, it is desired to analyze those fragments of the second binding partner that bind most tightly to the immobilized first binding partner. In such case, the probe is washed with one or more eluants prior to addition of energy absorbing molecules.

At this point, the probe is inserted into the interface of the tandem MS of the present invention, and fragments (typically peptides) of the second binding partner detected.

If the identify of the second (free) binding partner is known, the masses of the detected fragments can be compared with those predicted by applying the known cleavage rules of the fragmenting enzyme to the primary amino acid sequence of the second binding partner. In this fashion, each fragment can be identified, thus locating within the structure of the second binding partner those portions responsible for binding to the first binding partner.

Although, in theory, a single stage MS device can be used, in practice fragments other than those arising from the second binding partner will be present, confounding such analysis. Definitive identification in the usual case thus benefits from the high mass resolution and mass accuracy of the instrument of the present invention, and further often benefits from ms/ms analysis.

If the second (free) binding partner is not known, the partner can be identified by ms/ms analysis.

Typically, such analysis takes the form of selecting a first parent peptide in a first stage of MS, fragmenting the selected peptide, and then generating a fragment mass spectrum in a second stage of MS analysis. Fragmentation is done in the gas phase, preferably by collision-induced dissociation. In the preferred embodiment of the affinity capture tandem mass spectrometer of the present invention, CID is effected in q2 by collision with nitrogen gas at about 10⁻² Torr.

The fragment spectrum is then used to query sequence databases using known algorithms, such as that disclosed in Yates et al., U.S. Pat. Nos. 5,538,897 and 6,017,693, and that employed in Protein Prospector MS-TAG (http://prospector.ucsf/edu) module.

Putative identifications can be further verified by selecting a second parent peptide and repeating the approach, as necessary to confirm that all peptides derive from an identifiable parent.

Thereafter, once the second binding partner is identified, the nature of the intermolecular interaction can be studied as set forth above. The known cleavage rules of the fragmenting enzyme (or chemical, such as CNBr) are applied to the primary sequence of the now-identified second binding partner, and the empirically measured peptides mapped onto the theoretical digest, thus identifying the peptides that had bound to, and thus in the native molecule contribute to the binding to, the immobilized first binding partner. And as above, the experiment can be repeated with increasing stringency of wash to identify those peptides most tightly bound.

Other perturbations can be performed to elucidate further the nature of the intermolecular binding.

The elution characteristics of the eluant to wash the probe following fragmentation of the second binding partner can be altered to identify the fragments that contribute most strongly to the interaction, or to identify pH-dependent or salt-dependent contacts that contribute to binding.

The principle is of course well-known in the chromatographic and molecular biological arts: with increased stringency of wash (e.g., increased salt concentration, higher temperature), those fragments less tightly bound to the immobilized first binding will be eluted off the first binding partner. In the present geometry, such poorly binding fragments will elute off the probe and be lost from the subsequent mass spectrometric analysis. A series of experiments can thus be performed in which the probe, or identical counterpart probes, are washed at increasing stringency, thus creating a graded series of subsets of fragments of the second binding partner, in which each successive subset has a smaller subset of more tightly binding fragments.

As noted above, the first (immobilized) and second (free) binding partners can be interchanged, allowing the other partner's binding contacts to be elucidated.

A further useful perturbation is removal or alteration of post-translational modifications on one or both of the binding partners. For example, if the first binding partner is a glycoprotein, treatment with one or more specific or nonspecific glycosidases prior to, and/or after, binding of the second binding partner will help elucidate the contribution of sugar residues to the binding.

Analogously, where one of the binding partners is nucleic acid, treatment of the nucleic acid binding partner with nuclease after binding of the other binding partner can help identify critical binding residues.

The above-described approach to characterizing intermolecular interactions replaces the multi-platform, labor-intensive, insensitive techniques of the prior art with a single platform, streamlined, sensitive approach. The approach is applicable to a wide variety of different biological systems and problems.

As suggested above, the methods of the present invention can be used for epitope mapping—that is, to identify the contacts within an antigen that contribute to binding to antibody, T cell receptor, or MHC. The methods can be used to elucidate the nature of binding of biological ligands to their receptors, of transcription factors to nucleic acid, and of transcription factors to other transcription factors in a multiprotein complex.

Although particularly discussed above with respect to protein/protein interactions, the methods of the present invention can be practiced to elucidate the binding interactions between lectins and glycoproteins, protein and nucleic acid, and small molecules and receptors.

Particularly with respect to small molecule ligands, the methods can also be applied to the design of agonists and antagonists of known receptors.

Over the past decade, techniques have been developed for combinatorially generating large numbers of small molecules and for screening such molecules in various homogeneous and live cell assays for their ability to affect one or more biological processes. For example, homogeneous scintillation proximity assays can be used to screen combinatorial libraries for binding to a known receptor; digital image-based cellular assays can be used to screen compounds from combinatorial libraries for downstream effects, such as cytoplasmic/nuclear transport of receptors, changes in intracellular calcium distribution, or changes in cell motility.

Once such a lead compound is identified, however, a detailed understanding of the interaction of the small molecule with its receptor will facilitate intelligent design of molecules with improved pharmacokinetics and therapeutic index. The techniques of the present invention are well suited for such use.

If the small molecule provides a signal near that provided by the energy absorbing molecules, MS is performed with single ion monitoring looking only for the known mass for the combinatorial library component.

C. Improved Sequence Coverage from Proteolytic Fragment Mixtures

Often, proteins desired to be identified or sequenced by mass spectrometry are present in admixture with other proteins. Even those proteins first enriched by gel-based or liquid chromatographic approaches are rarely purified to homogeneity prior to MS analysis. For example, what appears by eye to be a single spot on a 2-dimensional PAGE gel can contain in excess of 10 different protein species that co-migrate to the same gel coordinates due to similar charge and mass properties.

The admixture of proteins complicates protein identification by mass spectrometry, whether such identification is to be performed by peptide mapping, using masses obtained, e.g., by matrix-assisted laser desorption ionization (MALDI) mass spectrometry, or is to be performed by tandem MS sequencing, using tandem MS spectra obtained, e.g., from liquid chromatography-mass spectrometry (LC-MS) tandem mass spectrometers.

One problem is that identification of proteins by mass spectrometry is substantially improved when a plurality of cleavage products of the protein can be sampled and the spectral data from the several cleavage products associated. In other words, identification improves with increasing collective sequence coverage.

For example, using virtual tryptic digests of bovine fetuin in database mining experiments, it has been demonstrated that even with an accuracy of 1.0 ppm (a level not currently achievable by most MS techniques), a poor confidence protein ID match is achieved using only a single peptide mass when searching against this complex, eukaryotic genome. For two peptides, low confidence results are achieved as well. Only after three peptides are submitted are confident results returned for mass assignments of less than 300 ppm error. With five or more peptides, no further confidence is afforded with mass accuracies better than 1000 ppm error. Merchant et al., Electrophoresis 21:1164-1167 (2000).

When proteins are present in admixture, however, it may prove difficult reliably to identify three, or four, or five cleavage products as having been derived from the same protein, thus confounding efforts at protein identification.

One solution to the problems caused by protein admixture is to perform further off-line purification prior to MS analysis. Typically, such purification is achieved using a column-based approach; this approach, however, can lead to loss of sample due to retention of samples on the column, on the separation media, and/or due to sample precipitation.

Another solution, described above as one aspect of the present invention, is to simplify the protein mixture on an affinity capture probe prior to cleavage of the protein mixture on the probe itself.

On occasion, however, the protein mixture has already been cleaved at the time mass spectrometric analysis is contemplated. For example, it is not uncommon to digest proteins that comigrate on a 2-D gel (i.e., that are detectable as a unitary spot) prior to their elution and subsequent analysis.

On other occasions, protein cleavage may not be a necessary concomitant of prior purification steps (such as elution from gels), but may nonetheless be desired prior to adsorption to the affinity capture probe. For example, one may wish to cleave proteins present in admixture prior to adsorption if on-probe cleavage is observed to be, or is expected to be, inefficient.

The prior cleavage of a protein mixture, by increasing the complexity of the mixture prior to analysis, presents further problems.

For example, standard matrix-assisted laser desorption/ionization-based approaches to protein identification are adversely affected by ion competition and quenching (suppression) effects; these effects are directly related to the total complexity of the adsorbed peptide mixture.

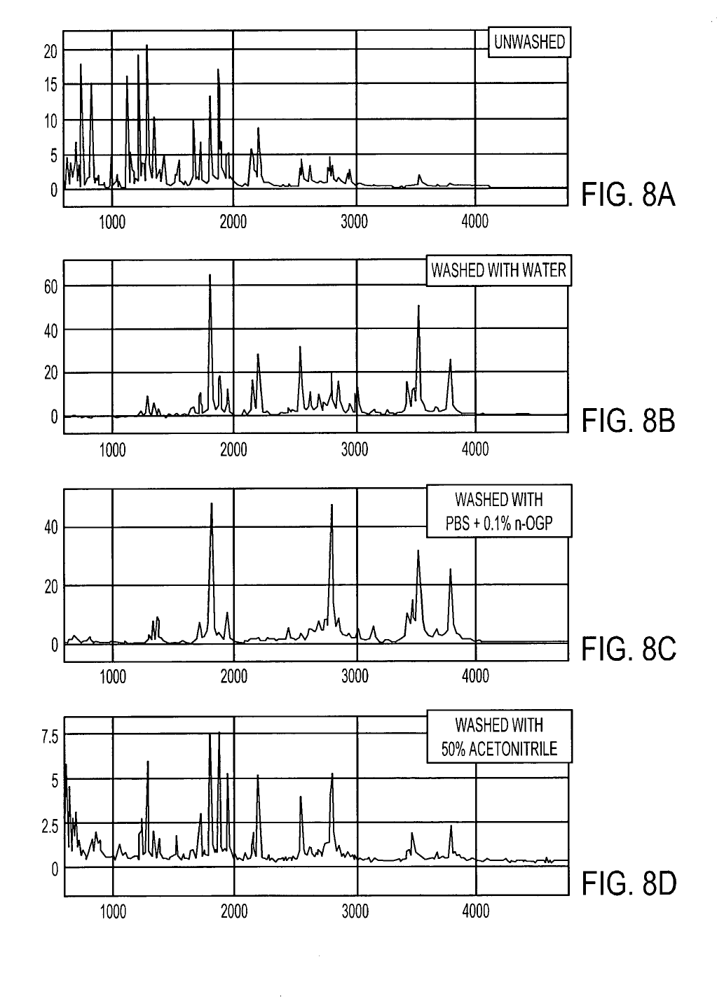

For example, FIG. 8A shows the mass spectrum obtained from a tryptic digest of IgG adsorbed to a reverse phase ProteinChip® Array (Ciphergen Biosystems, Inc., Fremont, Calif., USA). As can readily be seen, lower molecular weight peptides predominate; few peptides are seen in the upper MW ranges, due to ion competition from the lower molecular weight species. As further discussed in Example 2, below, the detectable peptides include only about 65% of the IgG sequence; that is, they collectively provide only about 65% sequence coverage.

Additionally, as the complexity of the mixture adsorbed to a MALDI probe increases, both the relative and absolute abundance of any one peptide typically decrease; this, in turn, decreases the signal to noise ratio, degrading the ability to acquire sequence from MS/MS analysis.

Furthermore, as the abundance of a peptide on the probe decreases, so too does the abundance of doubly charged ions created by laser interrogation; because doubly charged ions are a preferred ionic species for MS/MS sequencing, the decreasing abundance interferes with MS/MS sequencing efforts.

Thus, in another aspect, the invention provides methods for identifying a protein from its cleavage products, which cleavage products are present in admixture with cleavage products of other proteins. The methods increase the collective sequence coverage of proteolytic fragments of an analyte that can be detected by MS. The increased sequence coverage can improve protein identification and sequencing by tandem MS, which can advantageously be performed using the analytical device of the present invention.

In a first embodiment, proteins are already present as cleavage products in admixture with cleavage products of other proteins. The mixture of cleavage products is typically the result of prior cleavage of a protein mixture with a proteolytic agent; the protein mixture can, e.g., be an unpurified biological sample, a mixture of proteins that comigrate in a 2D gel, or a mixture of proteins eluting in a common chromatographic fraction. In a second embodiment, the method includes the antecedent step of proteolytic cleavage. In both embodiments, the proteolytic agent is typically an endoprotease with known cleavage specificity, such as trypsin.

A plurality of cleavage products from the mixture is then captured by adsorption to at least one adsorption surface of an affinity capture probe. The adsorption surface can be a chromatographic adsorption surface or a biomolecule affinity surface. The plurality of cleavage products adsorbed to the adsorption surface(s) of the probe includes at least one cleavage product of the protein analyte desired to be characterized.

Depending upon the complexity of the original mixture, the frequency of cleavage by the proteolytic agent, and the nature of the adsorption surface and the physical conditions during adsorption (e.g., temperature and ionic strength), the mixture of cleavage products adsorbed to the probe can have varying degrees of complexity.

Next, the probe is washed at least once with a first eluant. The probe is washed for a time and under conditions sufficient to decrease the complexity of the plurality of adsorbed protein cleavage products, the adsorbed cleavage products of reduced complexity including at least one cleavage product of the protein analyte desired to be analyzed. The wash can thus serve simultaneously to decrease the complexity of the adsorbed mixture and increase the relative concentration of at least one cleavage product of the protein analyte among the protein cleavage products remaining adsorbed to the probe.

Optionally, the probe can be washed at least once with a second eluant, the second eluant having at least one elution characteristic different from that of said first eluant, for a time and under conditions sufficient further to decrease the complexity of the plurality of adsorbed protein cleavage products, the adsorbed cleavage products of further reduced complexity including at least one cleavage product of the protein analyte desired to be analyzed.

Thereafter, energy absorbing molecules are applied, the probe interrogated, and at least one cleavage product of the protein analyte characterized by tandem mass spectrometry. The interrogation and characterization is performed in an analytical device having a laser desorption ionization source, a probe interface, and a tandem mass spectrometer.

Typically, the tandem MS measurement comprises: (i) desorbing and ionizing the protein cleavage products adsorbed on the probe, generating corresponding parent peptide ions; (ii) selecting a desired parent peptide ion in a first phase of mass spectrometry; (iii) fragmenting the selected parent peptide ion in the gas phase into fragment ions; and then (iv) measuring the mass spectrum of the fragment ions of the selected parent peptide ion in a second phase of mass spectrometry. Gas phase fragmentation is usefully effected by collision induced dissociation (CID). In the embodiment of the analytical instrument of the present invention depicted in FIGS. 1 and 2, such CID is effected in q2.

The fragment spectrum can then be used for protein identification.

In one approach to protein identification, the fragment spectrum is used to determine at least a portion of the amino acid sequence of the selected parent peptide ion. The sequence determination can be done, for example, by calculating differences in masses among fragment ions of a particular fragment series represented in the fragment ion mass spectrum, and correlating the mass differences with the known mass of amino acids, according to well-established algorithms.

Next, the partial sequence, often in conjunction with the mass of the parent peptide ion and optionally with the genus or species of protein origin, is used to query a protein sequence database. The query is performed with parameters that typically cause return of at least one protein identity candidate, identified based upon the closeness-of-fit calculated between the predicted protein sequence and sequences prior-accessioned into the database. The database can contain empiric protein sequences, protein sequences predicted from nucleic acid sequences, or nucleic acid sequences that are translated during execution of the query.

The protein identity candidate can then be validated; that is, the likelihood that the identity candidate returned by query of sequence databases is the same as the protein analyte desired to be identified from the mixture can then be assessed.

To assess the likelihood that the identity candidate is the same as the protein analyte, the (unfragmented) mass measured for the selected parent peptide ion is compared to the masses predicted for cleavage products that would be generated by cleaving the protein identity candidate with the proteolytic agent that had been used initially to cleave the proteins in the protein mixtures before adsorption to the probe. A match between one of the predicted masses and the measured parent peptide ion mass indicates an increased likelihood that the identity candidate is the same as the protein analyte.

When the measured parent peptide mass matches a mass predicted by in silico cleavage of the protein identity candidate, further validation of the putative identification can be performed by comparing the predicted masses to masses measured for cleavage products desorbed from the probe (i.e., parent peptide ions) other than the cleavage product that had originally been selected and fragmented. Additional matches as between predicted and measured masses indicates an increased likelihood that the identity candidate is the same as the protein analyte.

Conversely, when the measured mass matches none of the predicted masses, suggesting that the candidate identified in the database search is incorrect, the probe can be interrogated an additional time, selecting a different parent peptide ion in a first phase of mass spectrometry for subsequent fragmentation, fragment mass analysis, and database mining.

In another approach to protein identification, which can be used additionally or alternatively to the first approach, the fragment spectrum is used directly, without first establishing a partial sequence, to determine at least one protein identity candidate.

In this latter approach, the identity candidate is chosen from a sequence database based upon the closeness-of-fit between the empirically measured fragment ion mass spectrum and mass spectra that are predicted from sequences prior-accessioned into a sequence database. Such predicted spectra are either generated during the comparison or are prior-calculated and stored in a derivative database of predicted mass spectra. Proteins in the database can then be ranked based on the closeness of fit to the empiric fragment mass spectrum. Algorithms are known in the art to effect such a protocol. See, e.g., Yates et al., U.S. Pat. Nos. 5,538,897 and 6,017,693, the disclosures of which are incorporated herein by reference in their entireties.

As in the first approach, the mass of the parent peptide and/or protein analyte, optionally with information on the species of protein origin, can be used in the database query to facilitate and improve the reliability with which the protein identity candidate is chosen. For example, the taxonomic species of protein origin can be used as a filter to reduce the number of sequences for which predicted mass spectra must be calculated.

As in the first approach, the likelihood that the identity candidate is the same as the protein analyte can usefully be assessed. In such assessment, the (unfragmented) mass measured for the selected parent peptide ion is compared to the masses predicted for cleavage products that would be generated by cleaving the protein identity candidate with the proteolytic agent that had been used initially to cleave the proteins in the protein mixtures before adsorption to the probe. A match between one of the predicted masses and the measured parent peptide ion mass indicates an increased likelihood that the identity candidate is the same as the protein analyte.

When the measured parent peptide mass matches a mass predicted by in silico cleavage of the protein identity candidate, further validation of the putative identification can be performed by comparing the predicted masses to masses measured for cleavage products desorbed from the probe other than the cleavage product that had been selected and fragmented. Additional matches as between predicted and measured masses indicates an increased likelihood that the identity candidate is the same as the protein analyte.

Conversely, when the measured mass matches none of the predicted masses, suggesting that the candidate identified in the database search is incorrect, the probe can be interrogated an additional time, selecting a different parent peptide ion in a first phase of mass spectrometry for subsequent fragmentation, fragment mass analysis, and database mining.

The method of the present invention can be performed in any analytical instrument of the present invention, the instrument comprising a laser desorption ionization source, an affinity capture probe interface, and a tandem mass spectrometer. In particular, the tandem mass spectrometer can usefully be selected from the group consisting of QqTOF mass spectrometer, ion trap mass spectrometer, ion trap time-of-flight (TOF) mass spectrometer, time-of-flight time-of-flight (TOF-TOF) mass spectrometer, and Fourier transform ion cyclotron resonance mass spectrometer. Presently, a QqTOF MS provides certain advantages.

If the identification of the protein proves difficult or uncertain, the entirety of the procedure can be repeated on another aliquot of the protein mixture, using a different proteolytic agent and/or a different affinity capture probe having different adsorption surfaces.

And once identified, the protein analyte can advantageously be identified in further protein mixtures using affinity capture probes particularly chosen to effect substantial purification of the analyte cleavage products prior to tandem mass spectrometric analysis. Such particularly chosen affinity capture probes can usefully include at least one biomolecule affinity surface particularly adapted to capture the protein analyte through specific binding. For example, such biomolecule affinity surface can have antibodies or antigen-binding antibody fragments or derivatives specific for one or more cleavage products of the protein analyte, and can effect such specific binding with affinities desirably on the order of 10⁻⁶ M, more desirably 10⁻⁷ M, 10⁻⁸ M, and 10⁻⁹ M or better.

Although described particularly with respect to protein cleavage product mixtures eluted from 2D gels, the protein mixture can be derived from any biologic sample, including body fluids, such as blood, blood fraction, lymph, urine, cerebrospinal fluid, synovial fluid, milk, saliva, vitreous humor, aqueous humor, mucus and semen. The biological sample can equally be a cell lysate. The method requires only microliters of sample, and can be effected using submicroliter levels of sample, since nonspecific losses, as would be occasioned by fluid phase chromatographic purification, are obviated.

D. Proteolytic Amplification for Identification and Detection (“PAID”)

In another aspect, the invention provides methods for protein identification and detection in which protein fragments that correlate with a protein retained on an adsorption surface are used as markers in assays for proteins that are difficult to detect directly by mass spectrometry.

Proteins can be difficult to detect by mass spectrometry for a number of reasons. For example, some proteins possess modifications or primary attributes that can render their incorporation into matrix crystals problematic when compared to other proteins present within a complex mixture. Some proteins are more difficult to ionize when compared to other proteins found within a complex mixture. Furthermore, large proteins are generally more difficult to detect than small proteins because they are less efficiently converted to electrons at the ion detection surface.

Often, the more complex a sample, in terms of number of different proteins present, the more difficult it is to detect any particular protein in the sample. Proteins that comprise less than 10% of the total protein present in a sample frequently are difficult to detect. Therefore, methods to improve detection of these proteins are desirable.

In another aspect, therefore, the present invention provides methods for detecting proteins, particularly proteins that are difficult to detect by mass spectrometry. The methods involve the use of protein fragments of a target protein, which fragments have been identified by tandem MS, as protein fragment markers for the target protein. The method is particularly useful for detecting target proteins by single MS.

The target protein generally will be a known protein whose detection by single MS is difficult. To identify protein fragment markers that are useful in the method, the target protein is captured on an affinity capture probe.

Preferably, the affinity capture probe comprises a biomolecule affinity surface, such as an antibody, that specifically captures the target protein from the sample liquid. This greatly simplifies the analysis because, if a pure or substantially pure sample of the target protein is captured, all or most of the protein fragments generated will correspond with the target protein. However, affinity capture probes having chromatographic adsorption surfaces also are useful so long as they retain the analyte.

Whether adherent to a biomolecule affinity surface or to a chromatographic surface, the captured protein is fragmented by a reproducible fragmentation method. By reproducible fragmentation method is intended any method that would produce the same fragments when applied to a subsequent sample of the target protein. Such methods can be enzymatic or chemical.

In preferred embodiments, the target protein is fragmented by one or more proteolytic enzymes that cleave reproducibly at specific amino acid sequences, such as trypsin, clostripain, chymotrypsin or Staphylococcal protease, papain, thermolysin, pepsin, subtilysin, and pronase. Alternatively, fragmentation can be effected by treatment with a chemical agent that cleaves specifically. Examples of chemical agents that result in specific cleavage include, cyanogen bromide (CNBr), O-lodosobenxoate, hydroxylamine, and 2-nitro-5-thiocyanobenzoate, trifluoroacetic acid, pentafluroropropionic acid, or high concentration mineral acid solutions.

Fragmentation can be performed “on-chip” or in solution.

The resulting protein fragments are then analyzed by tandem MS to identify those that correspond with the target protein.

Typically, such analysis proceeds by selection, in a first phase of MS, of an ion of one of the protein fragments (parent peptide ion), fragmentation of the parent peptide ion in the gas phase (e.g., by collision-induced dissociation), and generation of a fragment ion spectrum in a second phase of mass spectrometry.

The fragment ion spectrum can then be used to determine the sequence of the parent peptide ion. As discussed elsewhere herein, which discussion is incorporated here by reference, such sequence determination can be performed by any or all of the methods known in the art, including de novo sequence determination, database mining using partial sequence, database mining using partial sequence and parent peptide ion mass, and database mining using closeness-of-fit of the fragment ion spectrum to theoretical spectra generated algorithmically from sequence databases. Since the identity of the target protein typically is known, such techniques will readily identify whether the selected fragment derives from the target protein, and is thus a suitable fragment marker for the target protein.

The tandem MS procedure can usefully be repeated for each fragment that can be desorbed and ionized from the affinity capture probe, often yielding a plurality of fragment markers that can be correlated with the target protein and that can thus be used in the method as surrogate markers for detecting the target protein in a complex mixture in subsequent target protein detection assays. The number of fragments used in a subsequent assay should be sufficient unambiguously to identify the target protein. In most cases, a single peptide marker is sufficient.

Once protein fragment markers are identified, an assay for the target protein in a test sample is performed as follows.

A test sample is exposed to the surface of an affinity capture chip that is known to capture the target protein. Preferably, this is the same type of adsorbent surface that was used to capture the protein from which the protein fragment markers were generated in the method above. Proteins in the sample are allowed to equilibrate on the chip and generally a wash is applied so that at least the target protein is retained, and other proteins are washed off. This simplifies the complexity of the sample. Then the captured proteins are subject to fragmentation by a method that will generate the protein fragment marker or markers from the target protein.

The fragmented proteins on the chip surface are now analyzed by mass spectrometry. In this case, the mass spectrometry need not be tandem MS, because the purpose of this step is to detect the protein fragment marker(s). Detection of the protein fragment markers in the sample indicates detection of the target protein in the sample. Preferably, a single protein fragment marker is used as a surrogate to identify the target protein. However, more than one target fragment marker can be used together. The detection of the protein fragment markers can be quantified so that the amount of the target protein in the sample is determined.

E. Differential Peptide Display for Quick Protein Identification (“QPID”)

The methods of this invention also are useful for identifying a target protein that is differentially displayed between two samples. In particular, the methods are useful in the examination of samples having a plurality of proteins in which a mass spectrum of the samples displays both commonly displayed proteins and differentially displayed proteins. Preferably, the proteins targeted for identification are uniquely detected, i.e., they are present in one sample and absent in the other. Less preferably, the display of the target proteins can be quantitiatively different between the two samples. The latter case is less preferred because subsequent to digestion of the proteins in the sample (as described presently), it is more difficult to reconcile the fragments generated with the target protein.

The method begins with two samples comprising different protein populations. Typically, the samples comprise an experimental sample and a control sample. Examples of sample pairs useful in these methods are: samples derived from healthy versus pathologic sources (useful for discovering diagnostic biomarkers), samples derived from animals or model systems subject to toxic versus non-toxic conditions (useful for discovering biomarkers for toxicology), and samples derived from drug responders versus drug non-responders (useful for discovering clinical stratification biomarkers).

Preferably, the samples are profiled by difference mapping through surface-enhanced laser desorption ionization, that is, by adsorbing the proteins on the adsorbent surface of a biochip and detecting the proteins adsorbed. Preferably, this process involves washing away unbound proteins with an eluant, as this results in chromatographic separation of the proteins in the sample and a reduction in complexity. Alternatively, if the samples have been pre-fractionated, they can be applied to the adsorbent surface and allowed to concentrate there, e.g., to drying. Less preferably, after the samples have been applied and equilibrium is reached, the excess liquid can be removed. After application of the sample, an energy absorbing material is generally applied to the probe surface and the bound proteins are detected by laser desorption/ionization mass spectrometry. By comparing the spectra of the two samples, either by eye or by computer, the differentially displayed target protein is detected according to molecular weight.

Then, aliquots of each sample are subjected to protein fragmentation. The method of fragmentation can be enzymatic or chemical.

Fragmentation preferably is performed “on-chip.” Although fragmentation can be performed in solution, this can complicate identification of the target protein because many more protein fragments will be generated.

Many techniques for protein fragmentation are known in the art: proteins are optionally fragmented enzymatically, chemically, or physically.

Fragmentation can be non-specific (i.e., random), specific (i.e., only at particular sites in a given protein), or selective (i.e., preferential). Physical fragmentation methods, such as physical shearing, thermal cleavage, or the like typically result in non-specific protein fragmentation. In contrast, enzymatic and chemical fragmentation methods may produce non-specifically or specifically cleaved peptide fragments from proteins in a sample. One method of chemical fragmentation is acid hydrolysis. Examples of chemical agents that result in specific cleavage include, cyanogen bromide (CNBr), O-lodosobenxoate, hydroxylamine, and 2-nitro-5-thiocyanobenzoate, trifluoroacetic acid, pentafluroropropionic acid, or high concentration mineral acid solutions.

In preferred embodiments, the proteins in a sample are fragmented by one or more proteolytic enzyme. Exemplary proteases suitable for use in the methods of the present invention are optionally selected from, e.g., aminopeptidases (EC 3.4.11), dipeptidases (EC 3.4.13), dipeptidyl-peptidases and tripeptidyl peptidases (EC 3.4.14), peptidyl-dipeptidases (EC 3.4.15), serine-type carboxypeptidases (EC 3.4.16), metallocarboxypeptidases (EC 3.4.17), cysteine-type carboxypeptidases (EC 3.4.18), omegapeptidases (EC 3.4.19), serine proteinases (EC 3.4.21), cysteine proteinases (EC 3.4.22), aspartic proteinases (EC 3.4.23), metallo proteinases (3.4.24), proteinases of unknown mechanism (EC 3.4.99), or the like. More specifically, the enzyme can be trypsin, clostripain, chymotrypsin or Staphylococcal protease, papain, thermolysin, pepsin, subtilysin,and pronase.

Additional processing is optionally utilized if proteins in a sample include multiple polypeptide chains and/or include disulfide bonds. For example, if a protein includes multiple polypeptide chains held together by noncovalent bonds (e.g., electrostatic interactions or the like), denaturing agents, such as urea or guanidine hydrochloride may be used to dissociate the polypeptide chains from one another prior to fragmentation. If a protein includes disulfide bonds, e.g., within a single polypeptide chain, and/or between distinct polypeptide chains, the disulfide bonds are optionally cleaved by reduction with thiols, such as dithiothreitol, -mercaptoethanol, or the like. After reduction, cysteine residues from disulfide bonds are optionally alkylated with, e.g., iodoacetate to form S-carboxymethyl derviatives to prevent the disulfide bonds from reforming.

In a preferred embodiment, the fragmentation proceeds by limited enzymatic or chemical digestion. Limited enzymatic or chemical digestion in the context of this invention means no more than five, preferably no more than 2, fragments. Limited proteolytic approaches have three major advantages: decreased protein identification (ID) time, increased protein ID sensitivity, and ultimately enabled multiple proteins to be identified from a mixture.

In most capturing experiments, more than one protein is captured on an affinity probe surface. If a conventional enzymatic digestion were carried out on the surface, each protein would generate multiple peptides. Peptide maps that are derived from multiple proteins complicate data mining for multiple protein identification. MS/MS analysis of each peptide then generates ions that allow the data mining and protein identification.

Using this strategy, no additional purification step is required to isolate and purify each individual protein from the mixture. Therefore, it decreases protein ID time and increases sensitivity. Also, lesser starting materials are required because just one unique peptide can be sufficient for protein ID. Furthermore, since aggressive proteolytic approaches are employed, proteins that are originally resistant to the conventional enzymatic digestion are now degradable. Finally, this approach enables multiple protein IDs from a protein mixture.

The protein fragments generated from each sample are then examined by mass spectrometry. By comparing the fragments detected, a difference map between the samples is generated which identifies protein fragments that are differentially detected in the sample comprising the target protein. At least some of the differentially displayed protein fragments must represent fragments of the differentially displayed target protein.

Then, identity candidates for at least one of the differentially displayed protein fragments are determined using the tandem MS methods described herein. The target protein is then correlated with an identity candidate. The correlation can be based on any information available to the investigator. However, the primary item of information is the molecular weight of the protein. The investigator will recognize that the predicted mass of any identity candidate represents the mass of a protein before any post-translational modification. If the target protein has a mass that corresponds with the mass of an identity candidate, the investigator can have high confidence that he or she has determined the identity of the target protein. If the mass of the target protein does not correspond with the mass of an identity candidate, the investigator must rely on other information as well. The mass of the target protein may be greater than or less than the mass of the identify candidate.

If the mass of the target protein is greater than the mass of the identity candidate, the structure of the identity candidate can be examined to determine the probability of post-translational modifications in the candidate protein, such as glycosylation or phosphorylation sites. Some protein databases are annotated, providing information about known sites of modification and typical forms of modification. Further confidence can be achieved by testing the target protein for the post-translational modification suspected. For example, if the one suspects that the target protein is glycosylated, the protein can be subjected to glycosidases and the digested protein can be examined to determine whether the mass now conforms to the identity candidate.

Furthermore, physico-chemical properties of the identify candidate can be used to increase confidence in a match. For example, if target protein binds to a hydrophilic biochip surface, the investigator can query whether the identify candidate also is expected to have hydrophilic properties under the retention conditions used to capture the target protein.

If the mass of the identity candidate is greater than the mass of the target protein smaller this implies that the target protein is a fragmentation product of the identify candidate. This theory can be tested in silico. Knowing the amino acid sequence of the protein fragment or fragments determined to be part of the identity candidate, one can query the amino acid sequence of the identify candidate to determine whether any contiguous sequence fragment of the identity candidate that includes these fragments corresponds to the mass of the target protein.

If no identity candidate can be correlated with the target protein within an acceptable level of confidence (generally at least 90%), then further examination of the target protein and the generated is warranted. As described above, all fragments generated from the identity candidate can be virtually “removed” from the spectrum. Then the identity of another remaining protein fragment can be determined, thereby generating another identify candidate for the target protein. The process can be repeated until an identify candidate is identified having the requisite level of confidence.

The following examples are offered solely by way of illustration and not by way of limitation.

### EXAMPLE 1

### Tandem MS Identification of a Prostate Cancer Biomarker

Traditionally, prostatic carcinoma is diagnosed via biopsy after discovery of elevated blood levels of prostate specific antigen (PSA). In normal males, PSA is present at levels of less than 1 ng/ml. For both BPH and prostatic carcinoma, PSA levels may be elevated to 4-10 ng/ml. Chen et al., J. Urology 157:2166-2170 (1997); Qian et al., Clin. Chem. 43:352-359 (1997). PSA is known to have chymotryptic activity, cleaving at the C-terminus of tyrosine and leucine. Qian et al., Clin. Chem. 43:352-359 (1997).

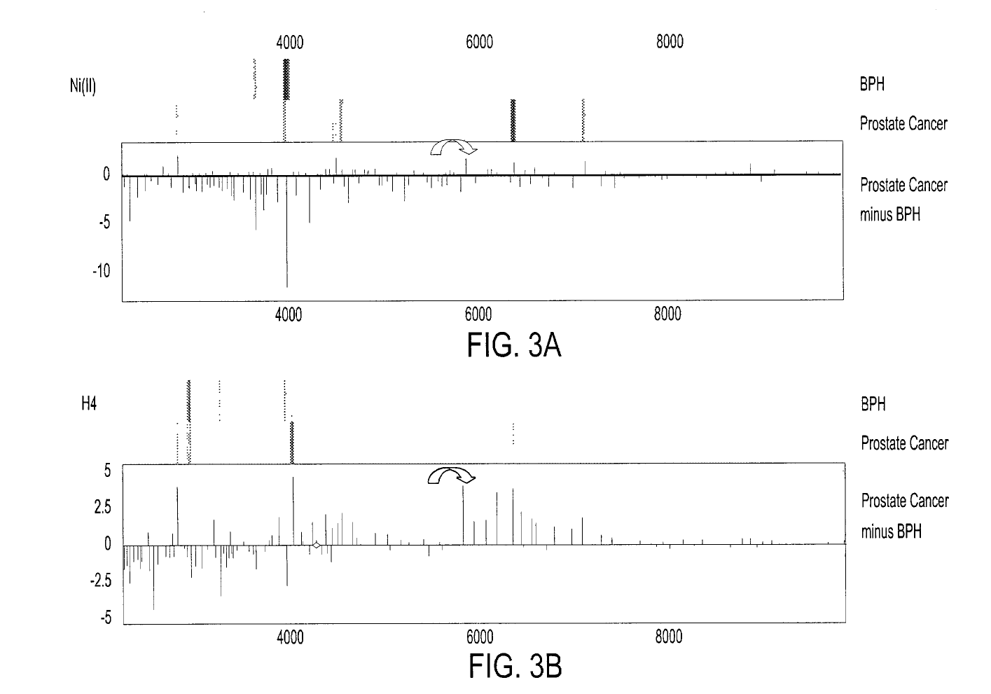

Seminal plasma from patients diagnosed with BPH as well as patients diagnosed with prostatic carcinoma were analyzed using the technique of ProteinChip® differential display. FIG. 3 displays the seminal fluid protein profiles of a single BPH and prostate cancer patient. A virtual gel display is used to enhance visual comparison between samples. A difference plot for the protein profiles of prostate cancer minus BPH is displayed beneath the gel view plots. Positively displaced signals of the difference plot indicate proteins that are upregulated in prostate cancer, while negative peaks represent prostate cancer downward protein regulation. Several uniquely upregulated signals, indicating possible prostate cancer biomarkers, were detected.

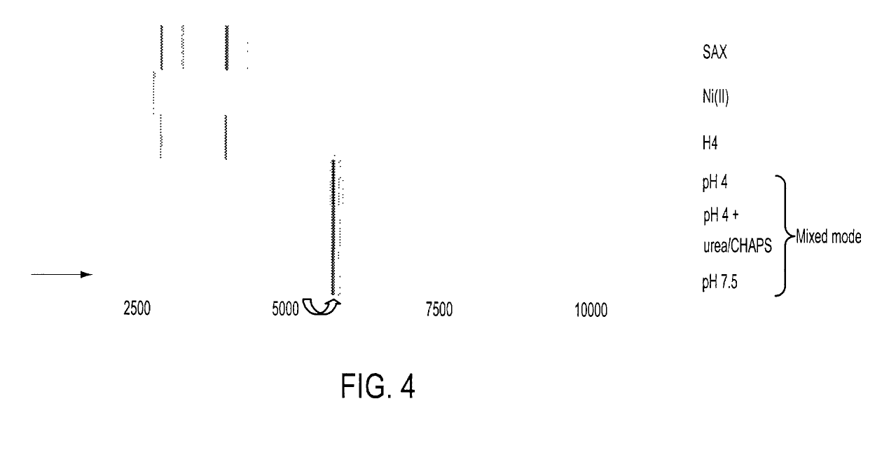

On-chip isolation of one of these upregulated proteins was achieved by using a mixed mode surface and neutral pH buffer wash (see FIG. 4). In this case, the protein was enriched to near homogeneity. The enriched biomarker candidate was then exposed to in-situ digestion using trypsin. After incubation, a saturated solution of CHCA (matrix) was added and the subsequent digest products analyzed by surface-enhanced laser desorption ionization time-of-flight mass spectrometry.

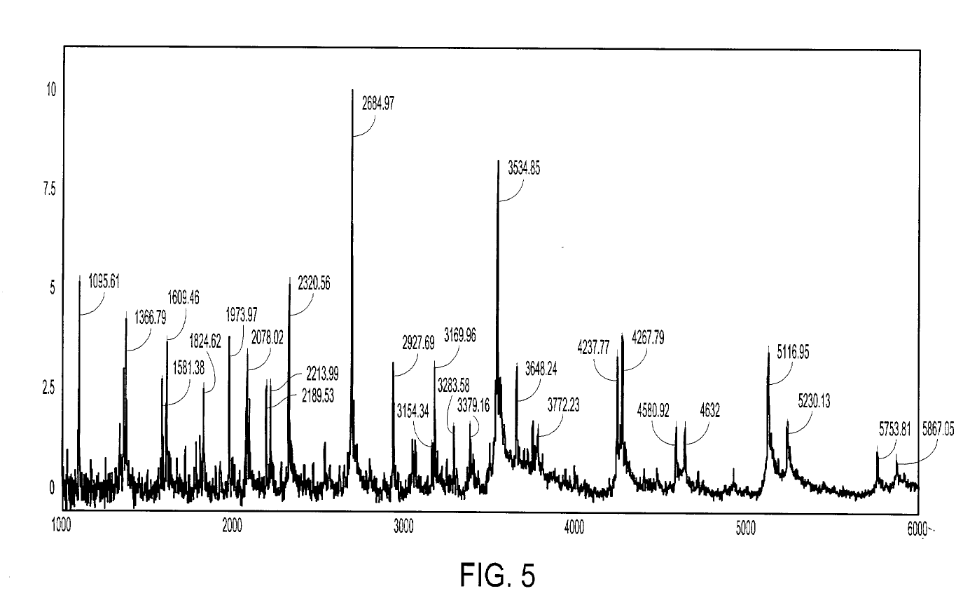

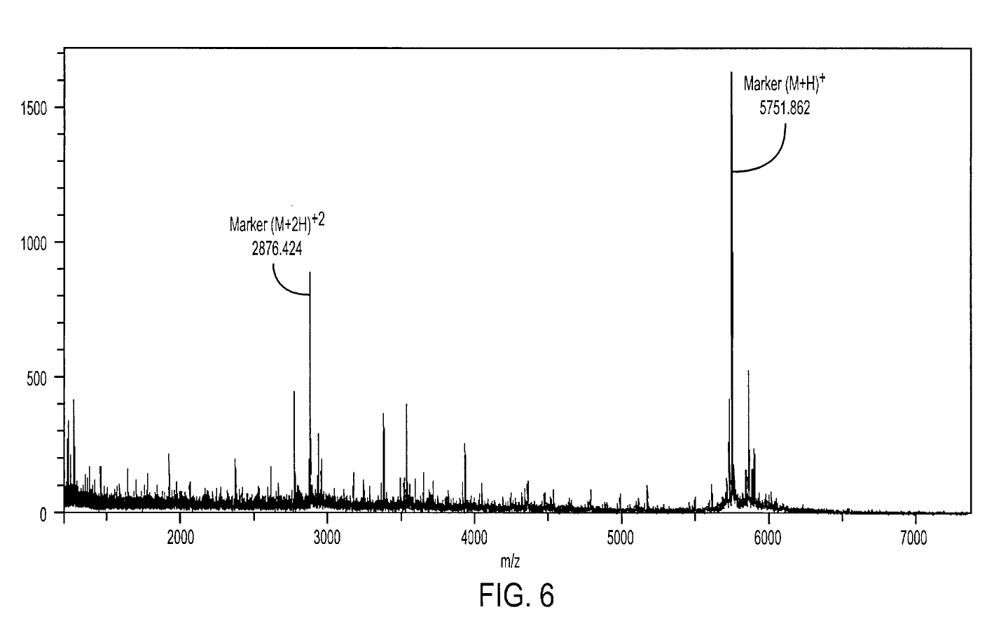

Several peptides were detected (see FIG. 5). The resultant peptide signals were submitted for protein database analysis and a preliminary identification of human semenogellin I was made. This identification was somewhat perplexing, since the candidate biomarker had a molecular weight by mass spectrometry of about 5751 Da, far less than that of semenogellin I (MW 52,131 Da).

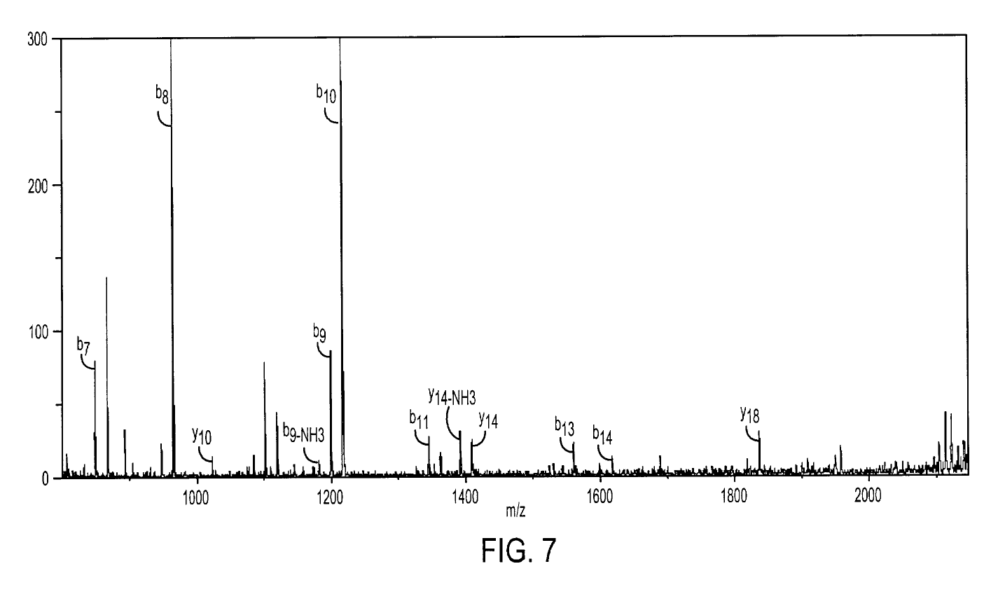

The same purified protein was submitted for ProteinChip LDI Qq-TOF MS detection (see FIG. 6). Because the parent ion at 5751 Da was beyond the current mass limit for LDI Qq-TOF MS/MS analysis (3000 M/z), the doubly charged ion was used for CID MS/MS sequencing (see FIG. 7). The CID MS/MS results were used to perform protein database mining. 15 of the 26 ms/ms ions mapped back to human seminal basic protein (SBP), a proteolytically derived fragment of semenogelin I, providing definitive identification of this candidate biomarker.

While initial studies such as these quickly reveal potential biomarkers, complete validation of any biomarker requires analysis of dozens or even hundreds of relevant samples to obtain statistically significant information regarding expression and prevalence.

### EXAMPLE 2

Increased Proteolytic Fragment Sequence Coverage For MS/MS Sequencing

To demonstrate that retentate chromatography on affinity capture probes can yield increased sequence coverage from proteolytic mixtures intended for MS/MS analysis, two experiments were performed.

In a first experiment, a complete tryptic digest was performed on a sample of IgG. The digest was then applied and allowed to adsorb to four identical, discrete, reverse phase chromatographic adsorption surfaces (“spots”) present on a single ProteinChip® array (Ciphergen Biosystems, Inc., Fremont, Calif., USA).

Prior to analysis, three of the four spots were washed. Energy absorbing molecules were then applied to each of the four spots and the spots separately interrogated in a single acceleration stage, linear time-of-flight mass spectrometer having a ProteinChip® Array probe interface (PBS I, Ciphergen Biosystems, Fremont, Calif., USA).

FIG. 8A shows the spectrum of the peptide mixture desorbed from the spot that had not been washed prior to analysis. As can readily be seen, lower molecular weight peptides predominate, suppressing desorption and ionization of the higher MW species. This can be a problem for peptide mapping and/or tandem MS sequencing techniques—particularly in cases where the sequence of the entire protein is desired or required—since the detectable peptides cover only about 65% of the primary IgG sequence.

FIG. 8B shows the spectrum resulting from desorption of peptides from another of the four spots, washed with water before laser interrogation. With elution of smaller, less hydrophobic, peptides prior to MS analysis, higher MW peptides become detectable. Similarly, FIG. 8C shows the spectrum resulting from desorption from a spot washed before interrogation with phosphate-buffered saline (“PBS”) containing the nonionic detergent n-octyl glucopyranoside (“n-OGP”) at 0.1%, and FIG. 8D shows the spectrum obtained by interrogation of the spot washed with 50% acetonitrile.

Comparing FIGS. 8A, 8B, 8C, and 8D, it is apparent that the differing wash conditions lead to the mass spectrometric detection of different collections of peptides from the same initial peptide mixture. Collectively, the differently washed spots provide peptides corresponding to more than 95% of the IgG sequence, demonstrating the power of this technique to increase collective sequence coverage among peptides to be used for MS/MS sequencing and protein identification.

In a second experiment, a complete tryptic digest of BSA, spiked with 2M urea, was analyzed under a variety of conditions.

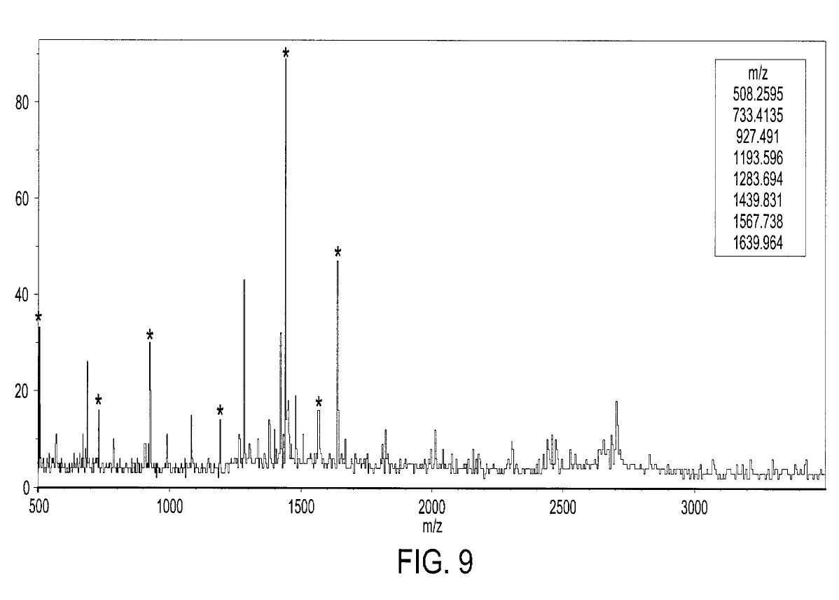

FIG. 9 shows the MS spectrum of a 2 μL aliquot of the digested BSA sample. The spectrum was aquired using a MALDI probe in a QqTOF MS. The spectrum demonstrates that only 8 peptides, providing 11% sequence coverage, could be detected. The m/z of the 8 peptides is separately tabulated at the right side of the figure.

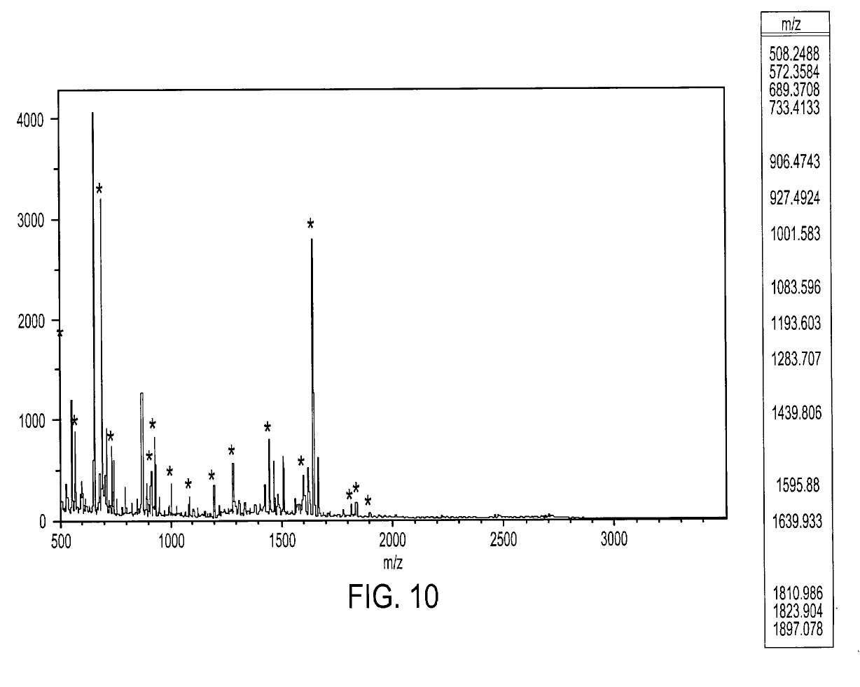

FIG. 10 shows the spectrum acquired from a parallel aliquot following its adsorption to an affinity capture probe having a weak cation exchange surface, with subsequent wash with buffer at pH 6. As can be seen, twice as many peptides are detected, collectively providing 20% sequence coverage. As in FIG. 9, the m/z of the detected peptides is tabulated at the right side of the figure.

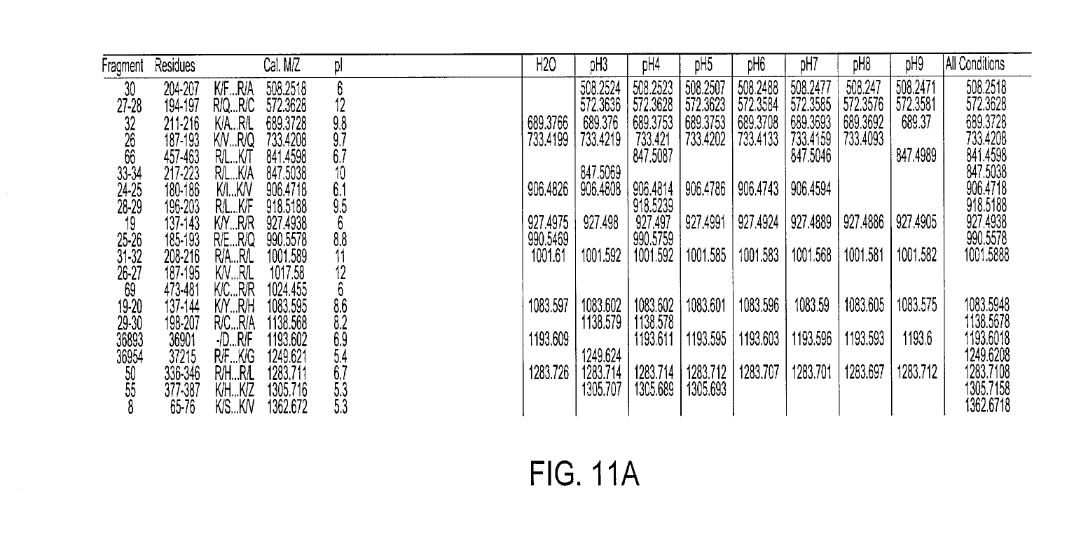

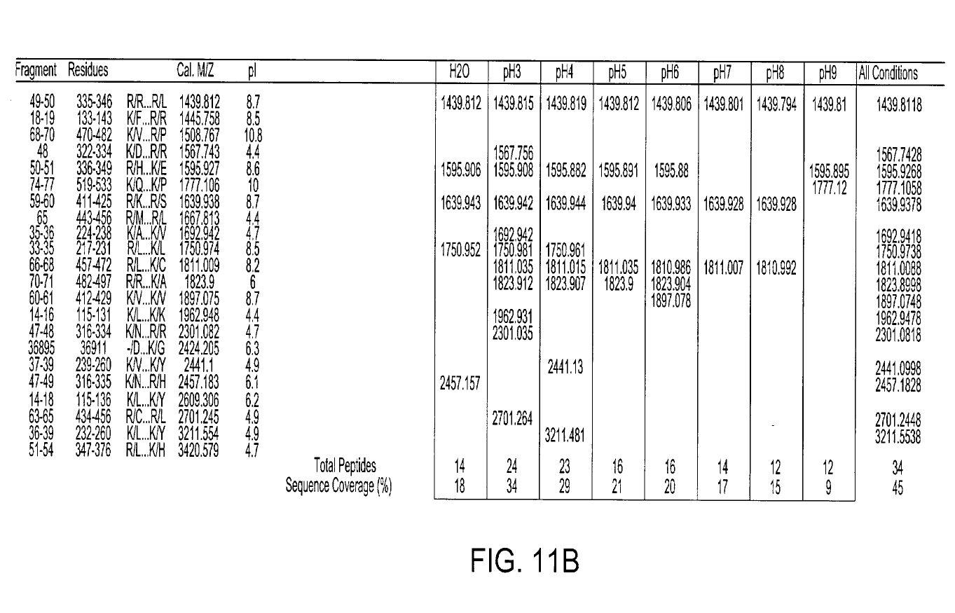

FIG. 11 compiles data from a series of experiments, including that shown in FIG. 10, in which aliquots of the same sample were applied to the weak cation exchange surface and washed under varying conditions prior to MS analysis. Collectively, the differing washes increase the number of peptides detected to 34, collectively providing 45% sequence coverage.

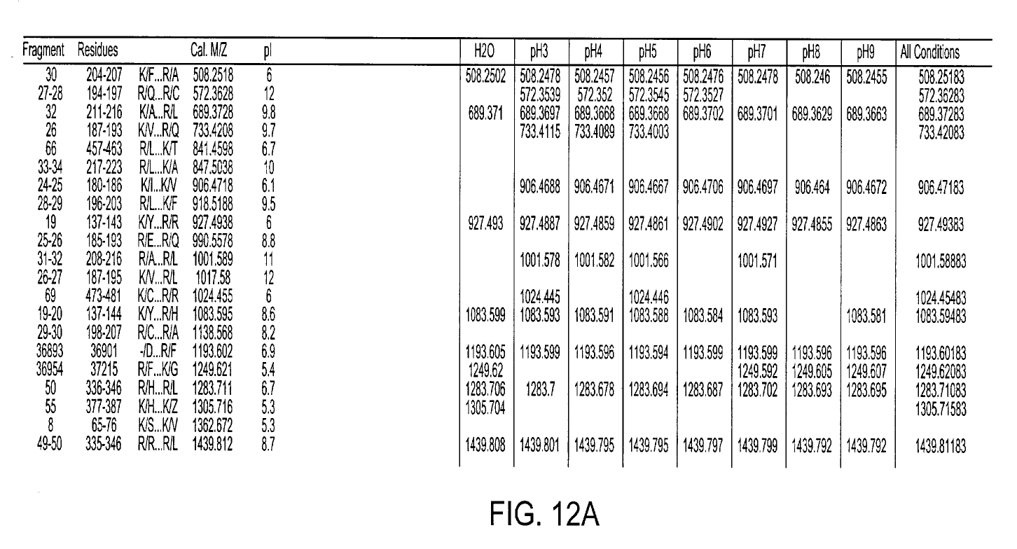

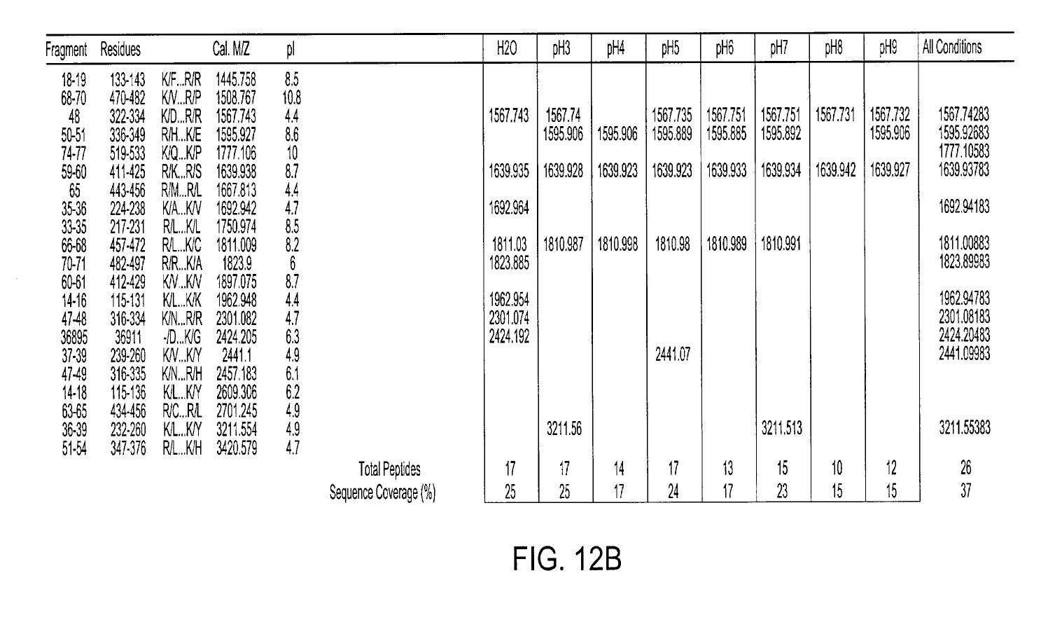

FIG. 12 compiles data from a series of experiments in which aliquots of the same sample were applied to an affinity capture probe having a strong anion exchange surface and thereafter washed under the indicated conditions prior to MS analysis. Collectively, the differing washes permit 26 peptides to be detected, collectively providing 37% sequence coverage.

Combining the data shown in FIGS. 11 and 12, 36 BSA peptides could be analyzed, collectively providing 46% sequence coverage. With such improvement in sequence coverage, subsequent MS/MS sequencing and/or sequence-based protein identification is substantially improved.

### EXAMPLE 3

### Proteolytic Amplification for Identification and Detection

A. Introduction

In this example, we used a CEA model system to show that:

1) protease digestion amplifies the detection of antigen up to 130 fold;

2) protein identification can be achieved using MS/MS analysis of one peptide from an on-chip digestion;

3) antibody capture and proteolytic amplification is quantitative within the range of the chip capacity; and

4) the detection limit of the antigen analyte in a complex protein mixture (antigen spiked into fetal calf serum) is at a level similar to the detection limit for pure antigen.

B. Materials and Methods

Antigen: Carcinoembryonic Antigen (CEA) was purchased from BioDesign International (Saco, Maine, Catalogue # A32137). Per the manufacturer, the protein had been purified from human fluids or human metastatic liver. CEA came in PBS buffer with 0.1% sodium azide at 2.5 mg/ml. It was diluted to 0.25 mg/ml by PBS and stored in aliquots at −20° C. CEA has 702 amino acids and a MW of 76.8 kDa. CEA is a glycoprotein and we observed a broad peak in MALDI around 150 kDa.

Antibody: Monoclonal anti-CEA antibody was also purchased from BioDesign (Catalogue # M37401M). It came in 0.9% NaCl at 2.3 mg/ml. It was stored in aliquots at −20° C.

Protocol for Antibody Capture and On-Chip Digestion:

Apply 2 μL of 1 mM protein G on all the spots of a Ciphergen Biosystems PS2 ProteinChip® array (the PS2 ProteinChip® has an epoxy surface which covalently reacts with amine and thiol groups, covalently binding protein G to the chip surface) and incubate the chip in humid chamber at room temperature for 2 hours. Residual active sites are blocked by placing the chip in a conical 15 ml tube with 8 ml of blocking buffer (0.5M ethanolamine in PBS, pH 8.0). The tube is mixed on a rotating platform for 15 minutes at room temperature.

After blocking, the chip is washed with 0.5% Triton X-100 in PBS for 15 minutes and then with PBS three times. The chip is air dried and 2 μl of anti-CEA antibody applied at 2.3 mg/ml to the desired spots. The chip is incubated in the humid chamber at room temperature for 2 hours. The chip is bulk washed with 0.5% Triton X-100 in PBS for 15 minutes and PBS three times.

Apply 2 μl of antigen at desired concentration to the spots. Incubate in the humid chamber at room temperature for 2 hours. Bulk wash the chip with 0.5% Triton X-100 in PBS for 15 minutes three times and followed by PBS wash three times. Let the chip air dry and apply 2 μl of pepsin at 0.01 mg/ml in 0.5% TFA. Incubate the chip in the humid chamber at 37° C. for 2 hours. Apply 1 μl of CHCA matrix on the digested spots and 1 μl of SPA on the undigested spots.

The chip was first read on a single MS, such as the Ciphergen Biosystems PBS II, and then on a tandem MS, such as a SELDI-QqTOF to obtain MS/MS spectra. Protein identification is then done, for example, by using MS-Tag.

C. Results and Discussion

1. CEA and anti-CEA Model Systems

Carcinoembryonic Antigen (CAE) is a glycoprotein that is expressed in a variety of secretory tissues. CEA is involved in the intercellular recognition and attachment involved in the development and proliferation of various metastases. Elevated serum levels of CEA are associated with several malignant states, and immunoassays for CEA have been used for several years in monitoring malignancy.

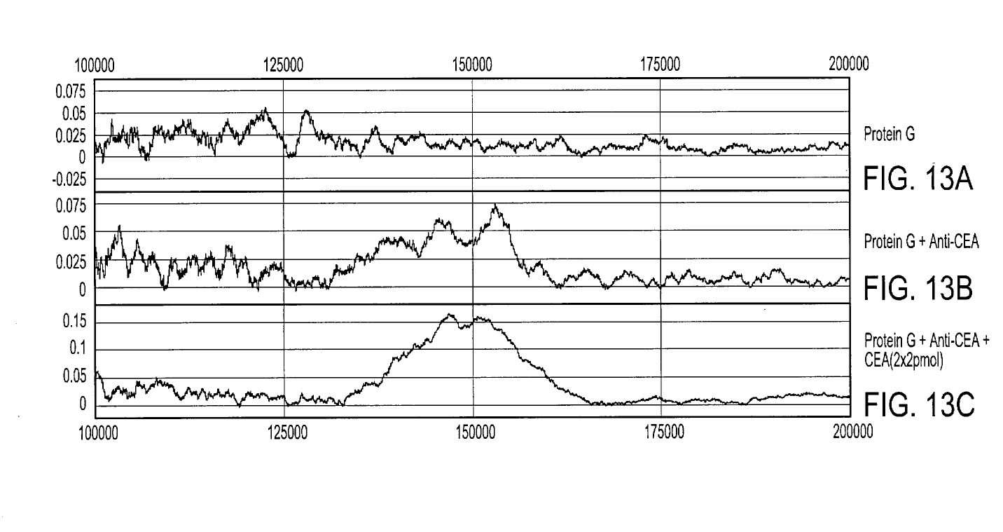

CEA was chosen as the model system for the following reasons: 1) CEA is hard to detect in MALDI due to its glycoprotein heterogeneity; and 2) CEA's molecular weight is around 150 kDa, which overlaps with that of the capture antibody. As shown in FIG. 13, anti-CEA is at 150 kDa with an intensity of 0.075. CEA captured by anti-CEA also has a signal around 150 kDa with the intensities between 0.1-0.2. It is, therefore, very difficult to prove that CEA is captured successfully without further identification.

1.1 Detection, Amplification and Identification of CEA Captured by Antibody

CEA was captured on PS2 chip by anti-CEA as described above. FIG. 13 shows mass spectra, generated using a Ciphergen Biosystems PBS II TOF-MS, at three stages in the preparation of the CEA chip: the top row shows the spectrum from the chip having protein G covalently bound thereto (“Protein G”); the middle row provides the spectrum from the chip further binding anti-CEA mAb (“Protein G+Anti-CEA”); and the bottom row shows the spectrum from the chip further binding 4 pmol CEA (“Protein G+Anti-CEA+CEA (2×2 pmol)”).

On the protein G+anti-CEA spot (middle spectrum), we observed a peak around 150 kDa, which is the antibody. As apparent from the protein G+anti-CEA+CEA spot (lowest spectrum), CEA captured by anti-CEA also has a signal at 150 kDa, with a slight increase in the intensity. The average intensity of the signal of CEA at 150 kDa is between 0.1-0.2. Since antibody is also at 150 kDa, we cannot draw the conclusion that CEA was captured.

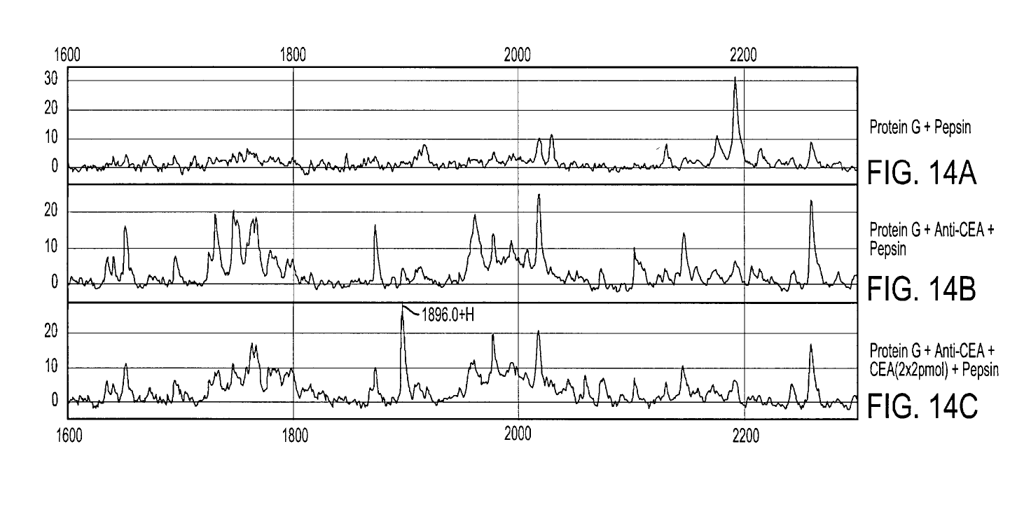

On-chip proteolysis was then performed to verify CEA was indeed captured and to amplify the signal of the CEA-reporting peak(s). FIG. 14 shows mass spectra, generated using a Ciphergen Biosystems PBS II TOF-MS, after on-chip pepsin digestion of the chips whose spectra are shown in FIG. 13. The top row is the spectrum from protein G+pepsin; the middle row is the spectrum from protein G+anti-CEA mAb+pepsin; the bottom row is the spectrum from protein G+Mab to CEA+4 pmol CEA+pepsin. As can be seen, M=1896 (labeled in the Figure) is unique in the CEA capture spot.

After the digestion, we found that anti-CEA antibody was also digested by pepsin (FIG. 14, row 2). We use this spectrum as the control. Comparing the digestion pattern of anti-CEA only (FIG. 14, row 2) and CEA captured by anti-CEA (row 3), we observed one major difference at mass 1896 (FIG. 14). TOF MS scan on the SELDI-QqTOF showed the accurate MH+=m/z 1894.9365.

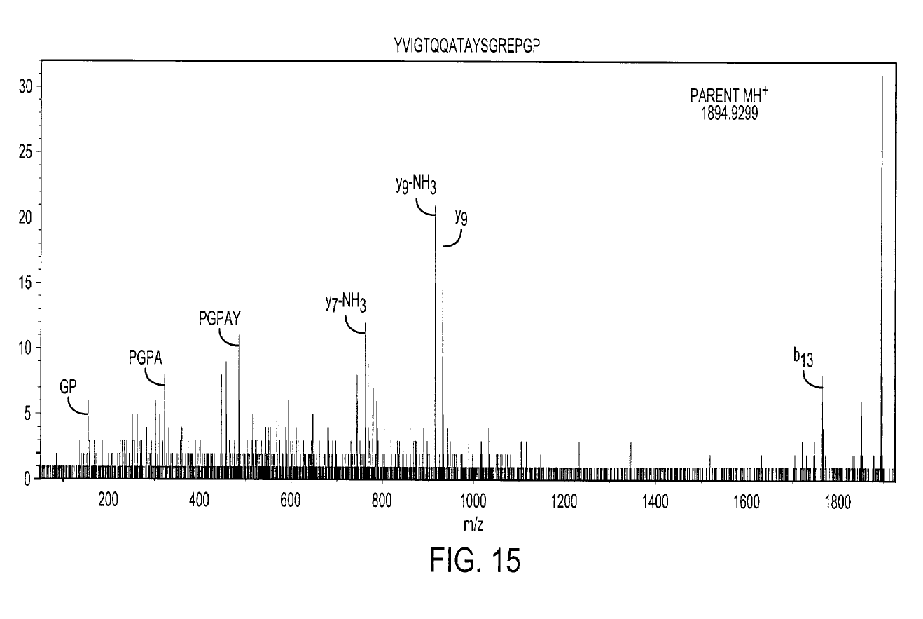

FIG. 15 shows the MS/MS spectrum of CEA peptide MH+m/z =1894.9299 obtained from using surface enhanced laser desorption ionization QqTOF. Peptide fragments arising from amide bond cleavage were observed corresponding to charge retention on the N-terminus (b ions), C-terminus (y ions) and internal fragments (labeled according to their sequence).

The fragments were submitted to MS-Tag for protein identification using the least stringent searching parameters (Molecular weight range: all; Species: all; Enzyme: none; parent ion: 20 ppm; fragment ions: 50 ppm; 640428 entries). This peptide was identified as peptide YVIGTQQATPGPAYSGRE from carcinoembryonic antigen.

The intensity of CEA at 150 kDa is 0.2, and the intensity of the reporter peptide at 1896 is 26. In this case we have observed 130-fold amplification of the CEA-reporting peak.

1.2 Quantitation of CEA Captured by Antibody

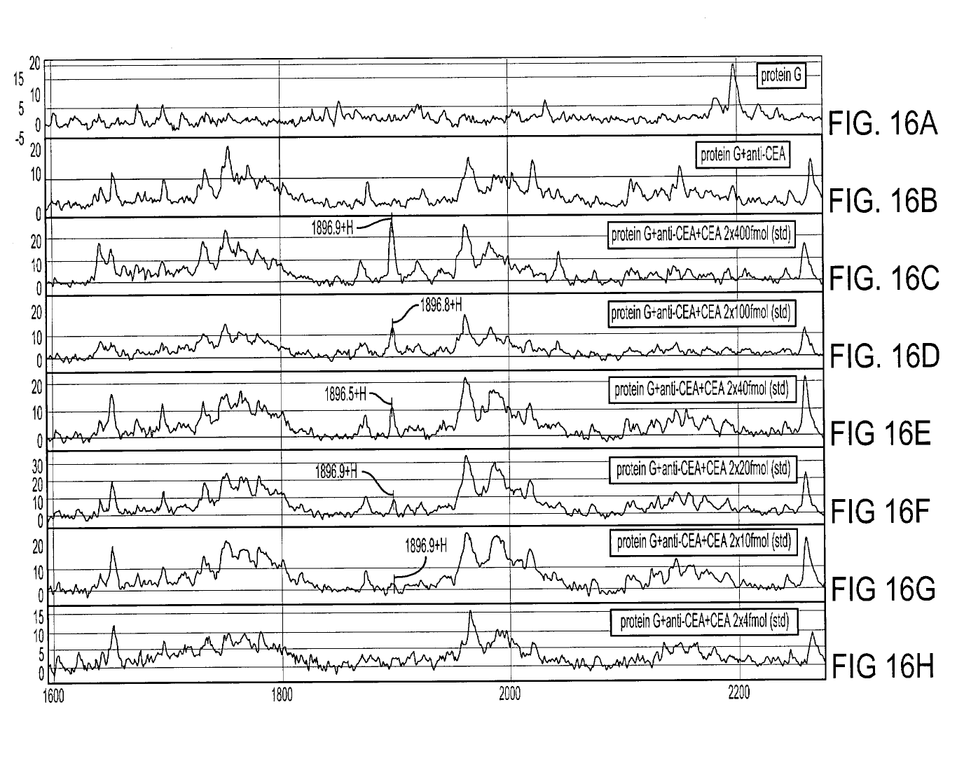

In order to assess the quantitative aspects of this assay, we performed a serial dilution of CEA from 400fmol/μl to 4 fmol/μl. 2 μl CEA was loaded on each spot. After pepsin digestion, an internal standard of 6 fmol somatostatin was spiked into the matrix. The spectra were normalized using somatostatin. FIG. 16 shows the spectra of the serial dilutions.

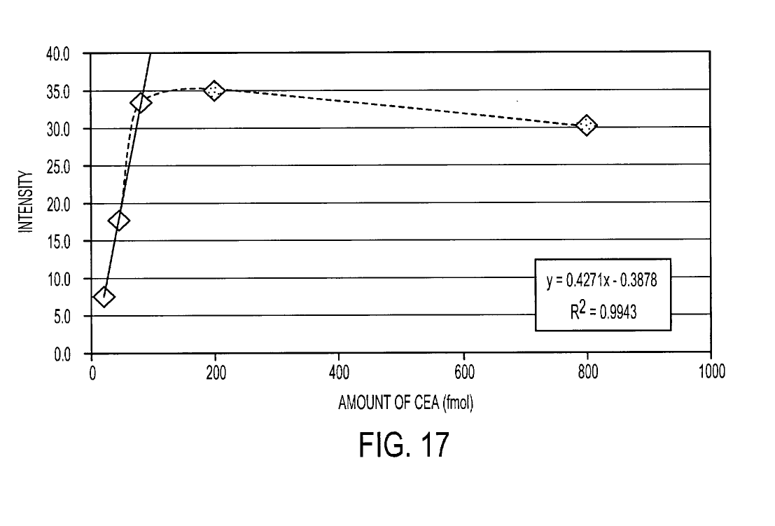

The intensities of the CEA-reporting peptide (mass=1896) were plotted against the amount of CEA loaded on the chip (FIG. 17). Linear response was observed from 20 fmol to 80 fmol; saturation occurred over 80 fmol. The solid line is the best linear fit of the first three data points with R²=0.9943. No reporter peptide was detected at 8 fmol level.

The quantitative results show, first, that the antibody capture of analyte (CEA) is quantitative over a certain range. The linear range depends on the chip capacity, antibody affinity and the detection limit for the antigen analyte or the reporting peptide. The results show secondly that the proteolytic digestion is quantitative within the same range.

1.3 Detection of CEA Captured by Antibody in the Presence of Fetal Calf Serum

CEA at the desired concentration was spiked into 30% fetal calf serum (fcs) in order to show the detection limit of CEA in the presence of a complex protein mixture.

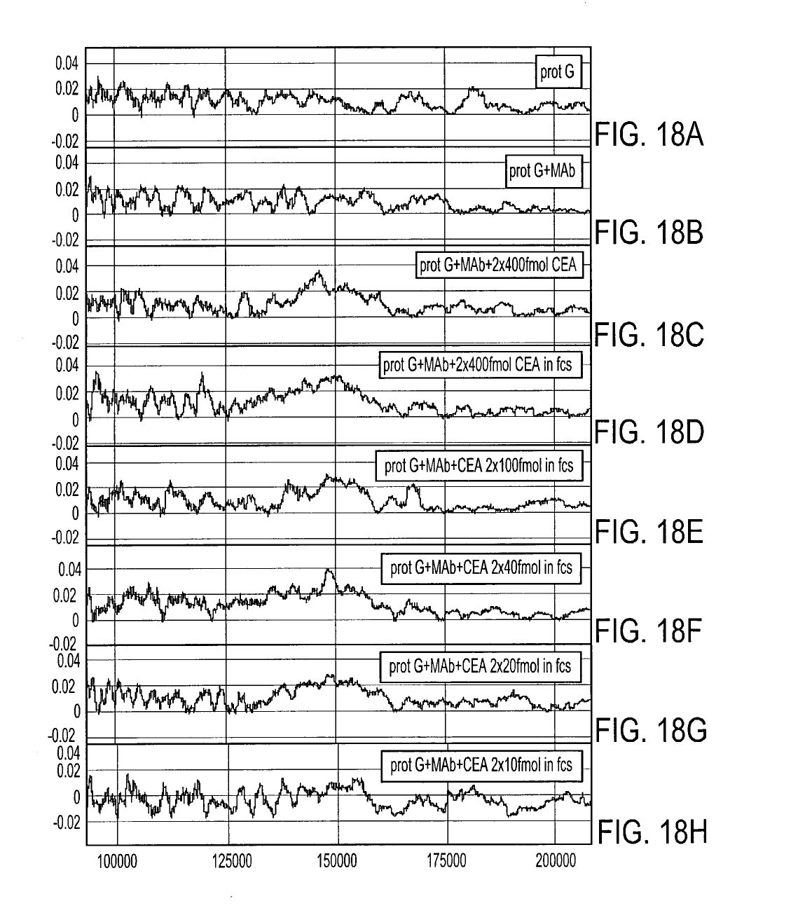

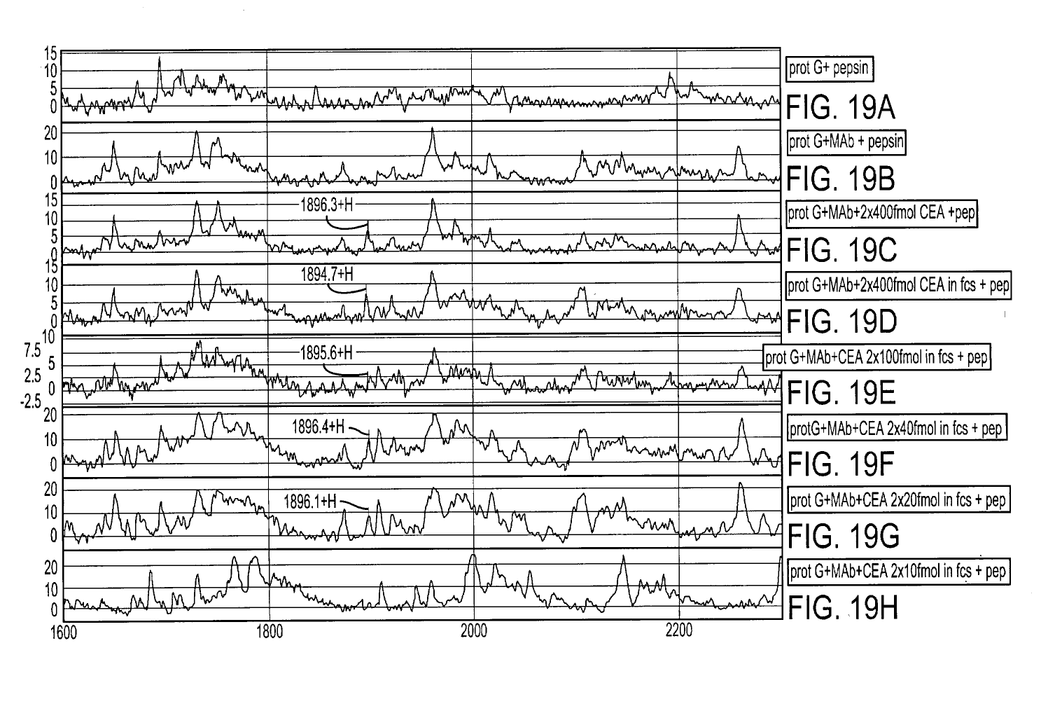

A serial dilution of CEA from 400 fmol/μl to 10 fmol/μl was prepared; 2 pl of CEA sample was loaded on each spot. Spectra are shown in FIG. 18. Non-specific binding of other proteins was observed (FIG. 18, 8 kDa, 10 kDa, 12 kDa and 38 kDa). Binding of CEA was detected at the 40 fmol level. The results are shown in FIG. 18. After proteolysis, the detection limit of CEA reporting peptide is also 40 fmol (FIG. 19). The peptide at m=1896 (labeled in FIG. 19) is the CEA-reporting peptide.

### EXAMPLE 4

### Differential Peptide Display for Quick Protein Identification (“QPID”)

Two examples were performed to demonstrate that differential display of a peptide that is correlated with a differentially expressed protein can be used for rapid protein identification.

A. Experiment 1: Differential Display of Peptides from Limited Enzymatic Digestion for Quick Protein Identification

1. Background

Tumor hypoxia is a pathophysiological state that distinguishes tumor cells from normal cells at the tissue level. The differences between hypoxic tumor cells and normal cells can be exploited to achieve therapeutic selectivity in cancer therapy. Furthermore, an understanding of the differences between hypoxic tumor cells and normal cells will be important in designing therapies that overcome or circumvent the obstacle to successful cancer treatment that tumor hypoxia at times presents.

To develop new biomarkers for the detection and prognosis of various human cancers, we have analyzed changes in protein secretion induced by hypoxia using Surface-Enhanced Laser Desorption/Ionization Time-of-Flight Mass Spectrometry (SELDI-TOF-MS).

2. Materials and Methods

FaDu cells (derived from squamous cell carcinoma) were grown in serum-free media under hypoxic or normal conditions for 24 hours. The media were isolated and concentrated for ProteinChip® analysis.

Before ProteinChip® array analysis, the media were diluted in binding buffer (100 mM Na Citrate, pH 3) to a final protein concentration of 0.5 mg/ml. Strong anionic exchange ProteinChip® arrays were used (SAX) for the sample analysis. In brief, the array surfaces were pre-equilibrated with binding buffer (5 μl) for 15 min before the application of diluted media (5 μl). After binding at room temperature for 30 min (with constant shaking), the samples were removed and the array surfaces were washed with 5 μl of washing buffer (binding buffer with 0.5 M NaCl, 0.1% OGP) three times at room temperature. After the last wash, the array surfaces were either under further process or ready for analysis. For the samples that were ready for analysis, the arrays were rinsed with HPLC grade water before adding 0.5 μl of saturated CHCA (diluted in 50% ACN and 0.5% TFA).

After protein profiling, the array surfaces were equilibrated with digestion buffer (50 mM ammonium bicarbonate, pH 7.8) 2 μl for 15 min. Trypsin (0.2 pg/ml) was added to the surface add incubated overnight in humidity chamber. After digestion, the trypsin was allowed to dry on the surface and 1 μl of saturated CHCA was added to the array surface before SELDI analysis.

The tryptic peptide maps of samples were calibrated using trypsin autolytic fragments as internal standards. After comparing the tryptic peptide maps from samples under normal and hypoxia conditions, unique tryptic peptide peaks were selected for MS/MS analysis or ProFound database search.

3. Results

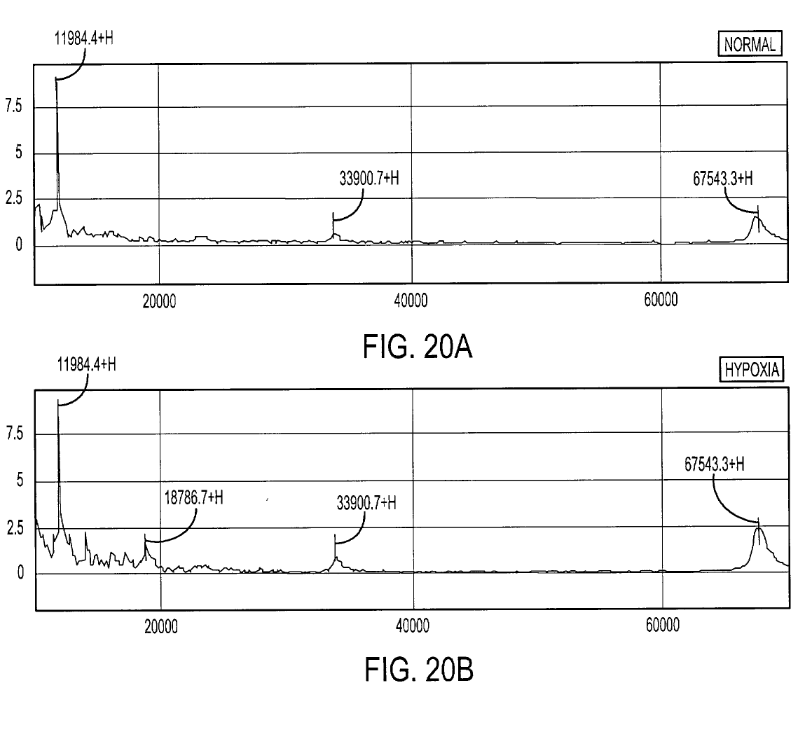

After comparing protein profiles of samples growing under hypoxic or normal conditions, a 18786.7 Da protein was shown to be strongly up-regulated in the samples treated under hypoxic conditions (FIG. 20). Under the experimental condition, the 18786.7 Da protein represents the major difference between the protein profile captured by SAX2 ProteinChip® surfaces. Three major protein peaks were observed in both samples at similar intensity were at 11984.4 Da, 33900.7 Da, and 67543.3 Da (FIG. 20).

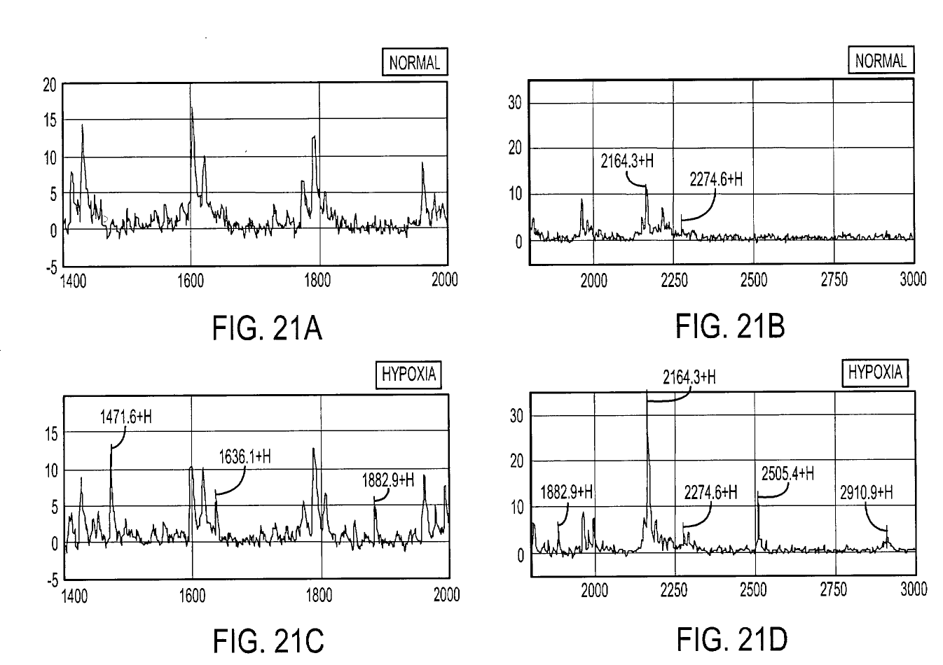

After trypsin digestion, five unique tryptic peptides (1471.60 1636.13 1882.89 2505.42 2910.89) were found in the samples treated under hypoxia conditions (FIG. 21). Two trypsin autolytic fragments (2164.3, 2274.6), found commonly in both samples, were utilized as standards for internal calibration. The five tryptic peptides were subjected to database query for protein identification. The same peptides can be subjected to MS/MS sequencing analysis as well.

ProFound database search returned several protein candidates using the unique tryptic peptide fragments. Zinc finger protein 9 (ZFP9), a 18.72 kDa human protein (Genomics 24:14-9 (1994)), was ranked at the top as the most probable candidate. ZFP9 is a member of a highly conserved family of cytosolic proteins called human cellular nucleic acid binding protein (CNBP). The function of CNBP is not known. CNBP was found in the cytosol and the endoplasmic reticulum in subcellular fractions, but was undetectable in nuclear fractions. Given the fact that we use the ProteinChip® array to capture secreted proteins in the cell culture media, the subcellular distribution and the molecular weight of ZFP9 suggest that it is a strong candidate for the 18.76 kDa protein captured by the ProteinChip® array.

B. Experiment 2: Peptide Differential Display for Quick Protein Identification

In a second experiment, 10 μl of cytochrome C (80 μg/ml=6.5 nmol/ml) was added (spiked) into 40 μl of 10% fetal calf serum (FCS) in phosphate buffered saline (PBS) (6 mg/ml). From this sample, 5 μl was spotted on an affinity capture probe having silicon oxide surface (NP20, Ciphergen Biosystems, Inc., Fremont, Calif., USA). In parallel, 5 μl of 8% FCS was spotted on an NP20 array. The NP20 arrays were incubated in a humid chamber for 15 minutes, and then bulk washed with 5 mM HEPES, pH 7.4 for 5 minutes. The wash was repeated two more times.

One μl of sinapinic acid (SPA) matrix (in 50% acetonitrile/0.5% TFA) was added to the array spots of one of the NP20 arrays, and this array then read in a Ciphergen Biosystems PBSII linear TOF mass spectrometer to obtain protein profiles.

The other NP20 arrays were loaded with two microliters of trypsin at 0.01 mg/ml in 100 mM NH₄HCO3, pH 8. They were then incubated in a humid chamber at 37° C. for 2 hours. One μl of CHCA matrix (in 50% acetonitrile/0.5% TFA) was added to the arrays. These arrays were read both in the PBSII linear TOF mass spectrometer and on a QqTOF tandem mass spectrometer (see FIGS. 1 and 2 for QqTOF schematics) to obtain differential peptide display and protein identification. Protein identification was done using MS-Tag (http://prospector.ucsf.edu).

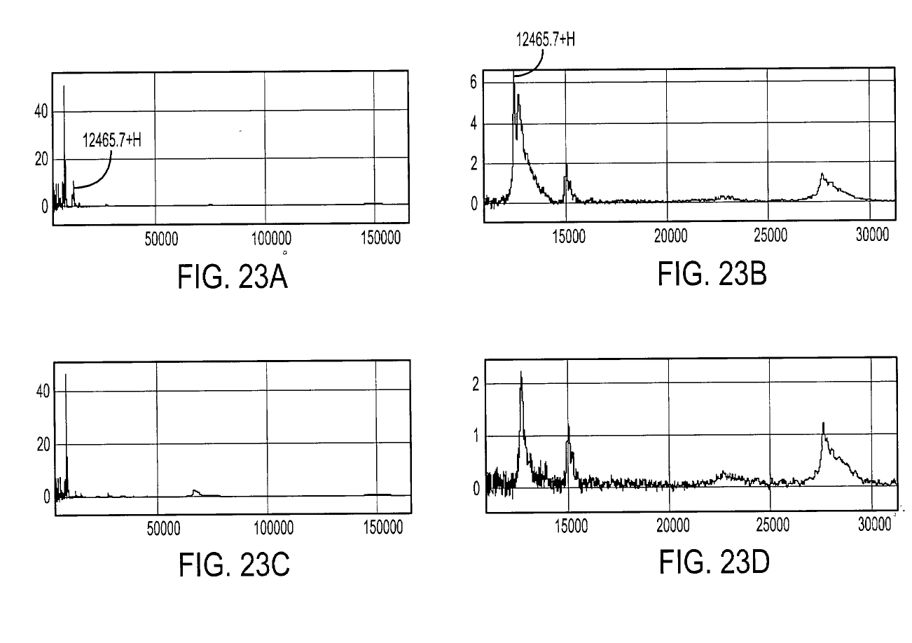

FIG. 23 shows the PBSII mass spectra (protein profiles) for sample (cytochrome C in FCS, panels A and B, with B at increased zoom) and control (FCS, panels C and D, with D at increased zoom). A peak uniquely appearing in the sample is marked (12465.7 daltons).

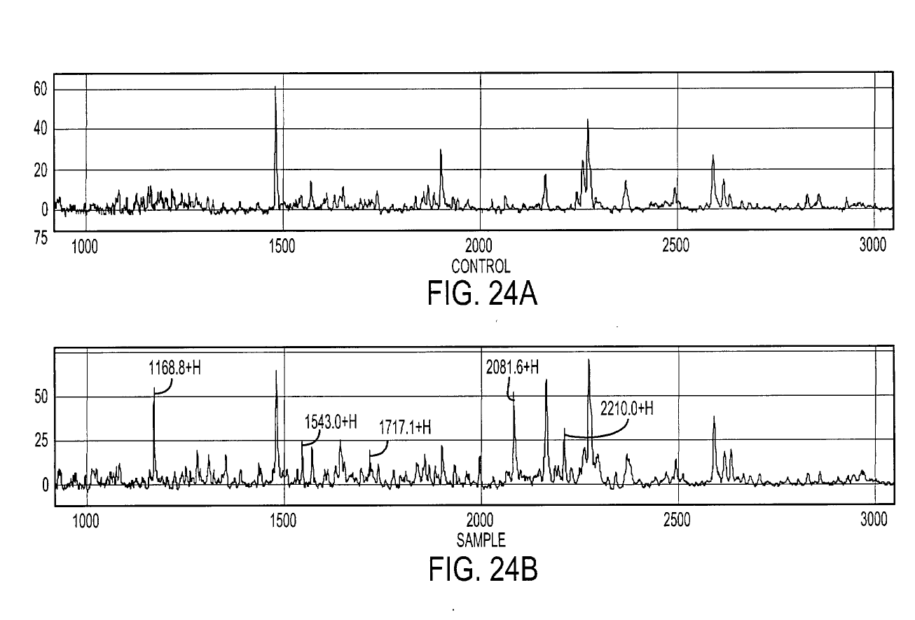

FIG. 24 shows MS spectra for sample and control acquired on the PBSII after on-chip digestion with trypsin. The spectrum at the top shows the control; the spectrum at the bottom shows the sample. Peptides that are uniquely present in the sample are labeled.

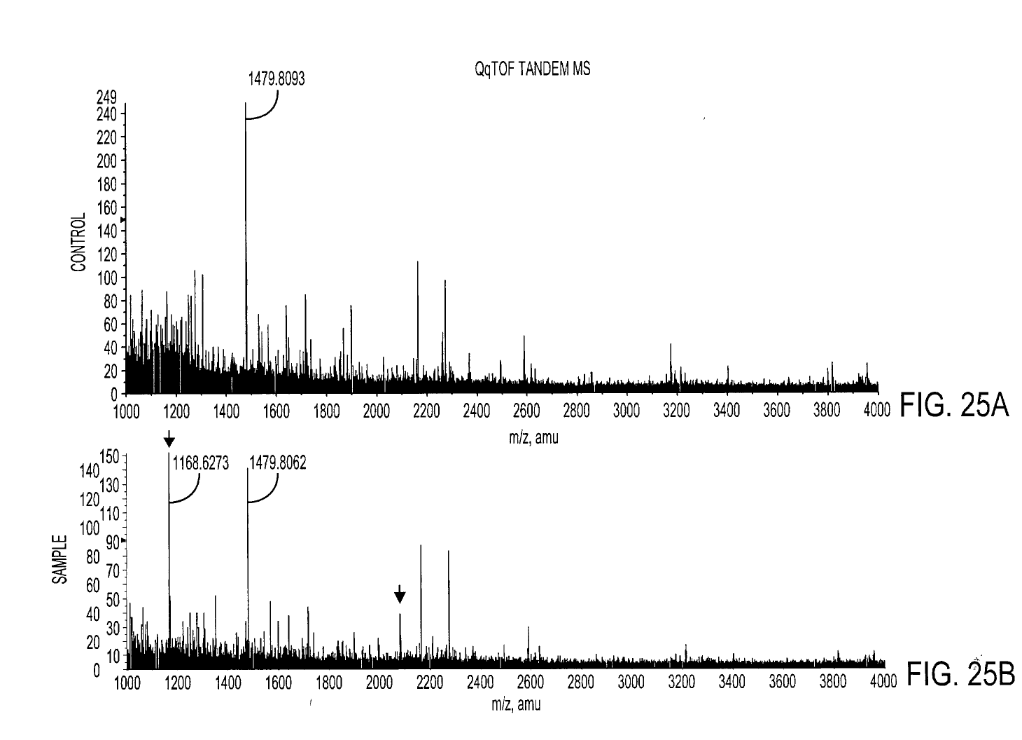

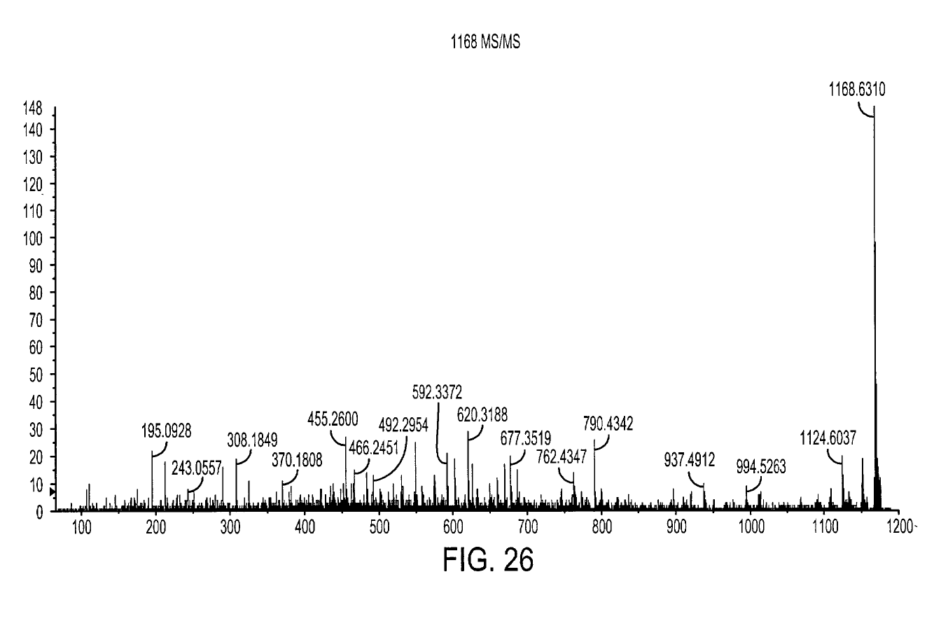

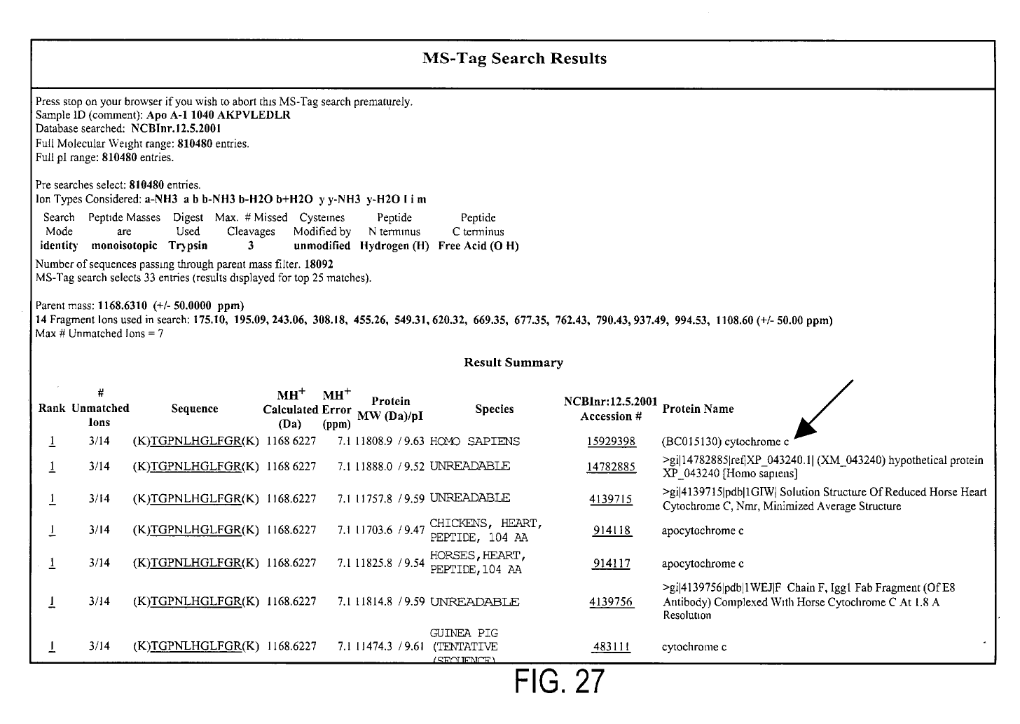

FIG. 25 shows spectra for sample and control, as in FIG. 24, but acquired on the QqTOF. The peptide at 1168 was then selected for CID and MS/MS analysis, with the resulting fragment spectrum shown in FIG. 26. Peptide fragment masses were submitted to MS-Tag, with results as shown in FIG. 27.

These results demonstrate that cytochrome C can be identified directly as a differentially displayed protein (FIG. 23) and can also rapidly be identified based upon the differential display of a constituent peptide following proteolytic digestion (FIGS. 26 and 27).

### EXAMPLE 5

### Limited Acid Hydrolysis

A. Limited Acid Hydrolysis

1. Background

In the past, complete acid hydrolysis of proteins was commonly used for amino acid analysis and partial acid hydrolysis was used for protein sequencing based on its ability to generate di- and tri- peptides. An inorganic acid, such as HCl, was usually the acid of choice, and proteins were usually treated at 110° C. with 2-6 M acid concentrations for several hours to a day.

Such hydrolytic conditions result in extensive non-specific cleavage; as a result, such conditions have limited value in protein identification endeavors using mass spectrometry, for some degree of cleavage specificity is required by most database mining algorithms. Accordingly, extensive acid hydrolysis approaches are deemed unsuitable for direct hydrolysis on the ProteinChip® array surfaces.

Recently, a vapor-phase acid hydrolysis method for mass spectrometric peptide mapping and protein identification has been reported. Lyophilized proteins were incubated in a sealed acid vapor chamber at 70° C. for 60 min. The bottom of the chamber was filled with 90% pentafluoropropionic acid (PFPA). Under these conditions, distinct types of cleavage reactions were observed: cleavage at specific internal amino acid residues (at the N-terminal side of serine, the C-terminal side of aspartic acid, and to a lesser degree at the N-terminal site of threonine and at both sides of glycine residues) and cleavages that result in the formation of sequence ladders containing the intact N- or C-terminus of the protein. Because of such specificity, vapor phase acid hydrolysis showed promise as being a viable technique for on-chip proteolysis to support database mining activities.

We performed limited acid hydrolysis using TFA (trifluoroacetic acid). We have investigated both vapor phase and solution phase acid hydrolysis. Our study showed that solution hydrolysis employing 6% TFA provided similar protein hydrolysis patterns as previously reported for gas phase reactions. For 6% TFA solution phase hydrolysis, preferred cleavage sites included both sides of glycine and the C-terminal side of aspartic acid. Furthermore, sequence ladders were often formed after the terminal peptides were produced. While using 0.6% TFA solution phase hydrolysis, observed cleavage patterns became more specific, with bond schism at the C-terminal side of aspartic acid being preferred. Applying solution phase TFA hydrolysis directly to ProteinChip array surfaces produced effective limited hydrolysis in an identical matter to that of free solution.

2. Methods

In the case of on-chip hydrolysis, 1-10 pmol of proteins were deposited on 8-spot mixed-mode, ProteinChip® arrays (Ciphergen Biosystems, Inc., Fremont, Calif.) and air-dried. Mixed-mode chips demonstrate mostly hydrophobic binding nature with some hydrophilic character. Then 2 μl of 6% TFA or 0.6% TFA (with 1% DTT) was added directly to each spot. Afterwards, chips were immediately put into a sealed humidity chamber (a plastic container employing a liquid reservoir). The bottom of the humidity chamber was filled with water and all chips were placed on a rack, suspended above the water surface. Then the humidity chamber was placed into a 65° C. oven. The reaction time for on-chip hydrolysis was generally 2-4 hours. After incubation, chips were taken out and the spots were air-dried prior to the addition of alpha-cyano-4-hydroxycinamic acid (CHCA) matrix solution.

A saturated solution of CHCA matrix was used for analysis of the acid hydrolysis products. The matrix solvent was 50%/50% H20/acetonitrile (v/v) and 0.5% TFA. Spectra were acquired in the positive-ion mode on a Ciphergen PBS II system (Fremont, Calif.), a time lag focusing, linear, laser desorption/ionization time-of-flight mass spectrometer. Time lag focusing delay time was set at 400 ns. Ions were extracted using a 3 kV ion extraction pulse, and accelerated to final velocity using 20 kV of acceleration potential. The system employed a pulsed nitrogen laser at repetition rates varying from 2 to 5 pulses per second. Typical laser fluence varied from 30-150 mJ/mm². An automated analytical protocol was used to control the data acquisition process in most of the sample analyses. Each spectrum was an average of at least 50 laser shots and externally calibrated against a mixture of known peptides. Peptide sequences could be directly derived from the mass spectrum when peptide ladders were generated. Protein sequences of our model systems were retrieved using NCBI database and Prowl software (http://prowll.rockefeller.edu/prowl/proteininfo.html).

3. Results

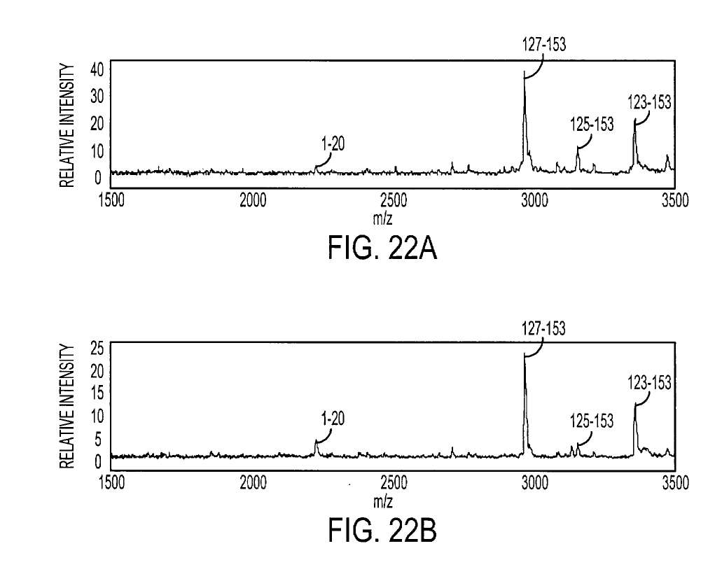

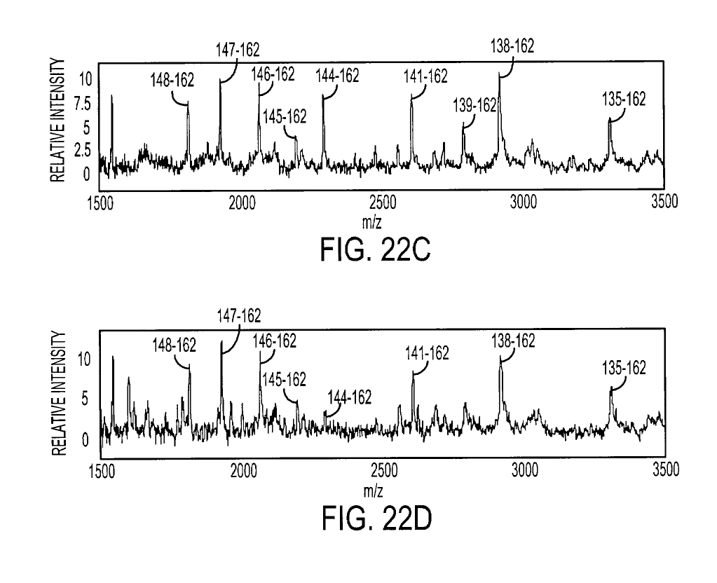

Results of on-chip acid hydrolysis experiments for apo-Mb and β-lactoglobulin A are depicted in FIG. 22, which depicts positive-ion mass spectra of peptide products resulting from 4 hr on-chip acid hydrolysis, as analyzed by the Ciphergen Biosystems PBS II MS, with conditions as follows:(a) 6% TFA, apo-Mb; (b) 0.6% TFA, apo-Mb; (c) 6% TFA, lysozyme; and (d) 0.6% TFA, lysozyme.

Surprisingly, similar hydrolytic patterns are observed for both high and low acid concentration experiments and in all cases hydrolytic fragments were seen within 60 minutes of incubation. Similar results were seen for BSA, lysozyme, and ribonuclease A. We believe the similarity of both low and high acid concentration hydrolysis products to be due to time-dependent dilution of on-chip acid solutions, making all experiments effectively proceed at low acid concentration. As all chips were incubated in a 65° C. humid chamber, with time the 2 μL acid solutions originally deposited to each position of the 8-position chip began to evaporate, thus loosing components in line with their respective vapor pressures. In essence, much of the TFA boiled off and a new equilibrium was established between the chip surface and surrounding gaseous media. For all experiments, the humid chamber fluid reservoir was typically loaded with about 180 mL of distilled water. Thus, effective TFA concentration for the on-chip droplet continually decreased, and after a complete exchange cycle, would be diluted by as much as four orders of magnitude.

The overall speed at which on-chip β-lactoglobulin A hydrolysis proceeded was also surprising. Compared to low acid concentration microcentrifuge tube results, β-lactoglobulin A on chip hydrolysis proceeded more rapidly, producing observable ladders within one hour in stead of requiring overnight incubation as was needed for microcentrifuge tube experiments. In this case not only did the observed cleavage pattern of both high and low concentration experiments resemble that of low concentration microcentrifuge experiments, but reaction rates were significantly increased. It is postulated that the ProteinChip array surface played an enabling role here by denaturing or presenting bound β-lactoglobulin A in a manner that improved access to acid labile residues.

Table 1 lists identified on-chip cleavage sites for all five proteins under high and low acid concentration conditions. Again, these products compare favorably with those generated by low concentration microcentrifuge tube trials, demonstrating preferred cleavage on the C-terminus of acidic residues. (For example: fragment 127-153 (D/A . . . G/_) from apo-Mb and fragment 135-162 (E/K . . . I/_) from bovine -lactoglobulin.) As was the case for microcentrifuge trials, on-chip acid hydrolysis reactions also demonstrated cleavage at asparagine and glutamine (for example: fragment 114-122 (N/P . . . V/_) from ribonuclease A and fragment 104-129 (N/G . . . L/_from lysozyme).

FIG. 22 depicts positive-ion mass spectra of peptide products resulted from 4 hr on-chip acid hydrolysis, as analyzed by the PBS II. (a) 6% TFA, apo-Mb. (b) 0.6% TFA, apo-Mb. (c) 6% TFA, lysozyme. (d) 0.6% TFA, lysozyme. The numbers indicate the amino acid range in the parent protein of the resulting fragment.

TABLE 1
Peptide products of on-chip acid hydrolysis
(both 6% TFA and 0.6% TFA)

| | | | | |
|---|---|---|---|---|
| Myoglobin | 2229.4 | 2230.4 | 1-20 | _/G . . . D/I |
| | 2970.3 | 2971.4 | 127-153 | D/A . . . G/_{—} |
| | 3360.9 | 3361.8 | 123-153 | D/F . . . G/_{—} |
| BSA | 1582.3 | 1583.7 | 1-13 | _/D . . . D/L |
| | 2805.5 | 2807.2 | 1-24 | _/D . . . L/I |
| B- | 1546.3 | 1545.7 | 150-162 | L/S . . . I/_{—} |
| lactoglobulin | 1815.7 | 1815.0 | 148-162 | I/R . . . /I_{—} |
| | 1928.6 | 1928.2 | 147-162 | H/I . . . /I_{—} |
| | 2066.3 | 2065.3 | 146-162 | M/H . . . I/_{—} |
| | 2198 | 2196.5 | 145-162 | P/M . . . I/_{—} |
| | 2294.3 | 2293.7 | 144-162 | L/P . . . I/_{—} |
| | 2405 | 2406.8 | 143-162 | A/L . . . /I_{—} |
| | 2479.6 | 2477.9 | 142-162 | K/A . . . /I_{—} |
| | 2607.1 | 2606.1 | 141-162 | L/K . . . /I_{—} |
| | 2721 | 2719.2 | 140-162 | A/L . . . /I_{—} |
| | 2791 | 2790.3 | 139-162 | K/A . . . /I_{—} |
| | 2919.9 | 2918.5 | 138-162 | D/K . . . /I_{—} |
| | 3311.3 | 3308.9 | 135-162 | E/K . . . /I_{—} |
| Ribonuclease | 1230.7 | 1230.4 | 114-124 | N/P . . . V/_{—} |
| A | 1662.3 | 1661.9 | 1-14 | _/K . . . D/S |
| Lysozyme | 1201.1 | 1201.5 | 120-129 | D/V . . . L/_{—} |
| | 2002.4 | 2002.4 | 1-18 | _/K . . . D/N |
| | 3048.8 | 3048.6 | 104-129 | N/G . . . L/_{—} |

**All the masses are average masses, as analyzed by PBS II.

4. Conclusions

For on-chip proteolysis studies (6% TFA or 0.6% TFA), the dominant preferred cleavage sites are at the C-terminal side of aspartic acid or deamidated asparagine and to a lesser degree the C-terminal side of glutamic acid or deamidated glutamine, followed by C-terminal cleavage at leucine. Under these limited conditions, a good degree of specificity is afforded and a reasonable rule set may be composed to create specific search algorithms to support database mining activity based upon 0.6%, limited acid hydrolysis.

All patents, patent publications, and other published references mentioned herein are hereby incorporated by reference in their entireties as if each had been individually and specifically incorporated by reference herein. By their citation of various references in this document, applicants do not admit that any particular reference is “prior art” to their invention.

While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

What is claimed is:

1. A method for detecting a target protein in a sample, comprising:
(a) capturing the target protein on an affinity capture probe;
(b) generating protein cleavage products of the target protein on the affinity capture probe using a proteolytic agent;
(c) detecting the protein cleavage products by laser desorption ionization mass spectrometry; and
(d) correlating one or more detected protein cleavage products with one or more prior-determined protein fragment markers of the target protein, whereby the correlation detects the target protein.

2. The method of claim 1 wherein the protein fragment markers are determined by:
(i) capturing the target protein on an affinity capture probe;
(ii) generating protein cleavage products on the affinity capture probe using a proteolytic agent;
(iii) analyzing at least one protein cleavage product with a tandem mass spectrometer, wherein analyzing comprises:
(1) desorbing the protein cleavage products from the affinity capture probe into gas phase to generate corresponding parent peptide ions,
(2) selecting a parent peptide ion for subsequent fragmentation with a first mass spectrometer,
(3) fragmenting the selected parent peptide ion under selected fragmentation conditions in the gas phase to produce fragment ions, and
(4) generating a mass spectrum of the fragment ions with a second mass spectrometer; and
(iv) identifying at least one protein fragment marker of the test protein from among the candidate protein cleavage products by:
(1) submitting at least one mass spectrum to a protein database mining protocol which identifies at least one protein identity candidate for the test protein in the database based on a measure of closeness-of-fit between the mass spectrum and theoretical mass spectra of proteins in the database; and
(2) determining whether the identity candidate corresponds to the test protein;
whereby a correspondence indicates that the protein cleavage product is a protein fragment marker of the test protein.

3. The method of claim 1 or claim 2 wherein mass spectrometry is laser desorption/ionization mass spectrometry.

4. The method of claim 3 wherein mass spectrometry is laser desorption/ionization time-of-flight mass spectrometry.

5. The method of claim 1 or 2 wherein the proteolytic agent is selected from the group consisting of chemical agents and enzymatic agents.

6. A method for identifying a protein that is differentially displayed between two complex biologic samples, comprising:
(a) detecting at least one protein that is differentially displayed between two samples with a mass spectrometer;
(b) fragmenting proteins in the two samples and detecting protein fragments that are differentially displayed between the two samples with a mass spectrometer;
(c) determining the identity of at least one differentially displayed protein fragment with a tandem mass spectrometer; and
(d) correlating the identity of the protein fragment with a differentially displayed protein, whereby the correlation identifies a differentially displayed protein.

7. The method of claim 6 wherein:
(a) detecting comprises:
(i) capturing proteins from the samples on affinity capture probe;
(ii) analyzing the captured proteins from each sample by laser desorption/ionization mass spectrometry;
(iii) comparing the captured proteins in the two samples to identify proteins that are differentially expressed;
(b) fragmenting and detecting comprises:
(i) capturing proteins from the samples on affinity capture probes;
(ii) generating protein cleavage products on the affinity capture probes using a proteolytic agent;
(iii) analyzing the protein cleavage products by laser desorption/ionization mass spectrometry;
(iv) comparing the protein cleavage products in the two samples to identify protein cleavage products that are differentially expressed; and
(c) determining the identity of at least one differentially displayed protein fragment comprises:
(i) desorbing the protein cleavage products from the protein biochip into gas phase to generate corresponding parent peptide ions,
(ii) selecting a parent peptide ion for subsequent fragmentation with a first mass spectrometer,
(iii) fragmenting the selected parent peptide ion under selected fragmentation conditions in the gas phase to produce product ion fragments with a second mass spectrometer,
(iv) generating a mass spectrum of the product ion fragments; and
(v) identifying at least one protein identity candidate fragment marker products by submitting at least one mass spectrum to a protein database mining protocol which identifies at least one protein identity candidate for the differentially displayed protein in the database based on a measure of closeness-of-fit between the mass spectrum and theoretical mass spectra of proteins in the database.

8. The method of claim 6 wherein fragmenting is performed in solution.

9. The method of claim 6 or 7 wherein the differentially displayed protein is detectable uniquely in one of said two samples.

10. The method of claim 6 or 7 wherein (b) fragmenting comprises enzymatic fragmentation.

11. The method of claim 10 comprising limited enzymatic digestion.

12. The method of claim 6 or 7 wherein (b) fragmenting comprises chemical fragmentation.

13. The method of claim 12 wherein chemical fragmentation comprises acid hydrolysis.

14. The method of claim 6 or 7 wherein the two samples are selected from (1) a sample from a healthy source and a sample from a diseased source, (2) a sample from a test model exposed to a toxic compound and a sample from a test model not exposed to the toxic compound or (3) a sample from a subject that responds to a drug and a sample from a subject that does not respond to the drug.

15. A method for analyzing a protein analyte present as a plurality of cleavage products in admixture with cleavage products of other proteins, comprising:
(a) capturing a plurality of cleavage products from said mixture by adsorption to an affinity capture probe, said plurality of adsorbed cleavage products including at least one cleavage product of said protein analyte;
(b) washing said probe at least once with a first eluant for a time and under conditions sufficient to decrease the complexity of said plurality of adsorbed protein cleavage products, said adsorbed cleavage products of reduced complexity including at least one cleavage product of said protein analyte; and then
(c) characterizing said at least one cleavage product of said protein analyte with a tandem mass spectrometer measurement,
said tandem mass spectrometric characterization of said at least one cleavage product providing an analysis of said protein analyte.

16. The method of claim 15, further comprising the antecedent step of:
cleaving proteins in said mixture into cleavage products with a proteolytic agent.

17. The method of claim 15 or claim 16, further comprising at least one iteration of the step, after washing with said first eluant and before characterizing said at least one protein analyte cleavage product, of:
washing said probe with a second eluant, said second eluant having at least one elution characteristic different from that of said first eluant, for a time and under conditions sufficient further to decrease the complexity of said plurality of adsorbed protein cleavage products, said adsorbed cleavage products of further reduced complexity including at least one cleavage product of said protein analyte.

18. The method of claim 15, wherein said characterizing with a tandem mass spectrometer measurement comprises:
i) desorbing and ionizing said protein cleavage products from said probe, generating corresponding parent peptide ions;
ii) selecting a desired parent peptide ion in a first phase of mass spectrometry;
iii) fragmenting said selected parent peptide ion in the gas phase into fragment ions; and
iv) measuring the mass spectrum of the fragment ions of said selected parent peptide ion in a second phase of mass spectrometry.

19. The method of claim 18, wherein said fragmenting is effected by collision induced dissociation (CID).

20. The method of claim 19, further comprising:
(d) determining at least a portion of the amino acid sequence of said protein analyte by calculating differences in masses among fragment ions represented in said fragment ion mass spectrum.

21. The method of claim 20, further comprising:
(e) determining at least one protein identity candidate for said protein analyte based upon the closeness-of-fit calculated between said predicted sequence and sequences prior-accessioned into a sequence database.

22. The method of claim 21, further comprising:
(f) assessing the likelihood that said identity candidate is the same as said protein analyte by comparing (i) the mass measured for said selected parent peptide ion to (ii) the masses predicted for cleavage products that would be generated by cleaving said identity candidate with said proteolytic agent,
a match as between a predicted mass and said measured mass indicating increased likelihood that said identity candidate is the same as said protein analyte.

23. The method of claim 15, wherein said tandem mass spectrometric characterization is performed using a mass spectrometer selected from the group consisting of QqTOF mass spectrometer, ion trap mass spectrometer, ion trap time-of-flight (TOF) mass spectrometer, time-of-flight time-of-flight (TOF-TOF) mass spectrometer, and Fourier transform ion cyclotron resonance mass spectrometer.

24. The method of claim 23, wherein said tandem mass spectrometer is a QqTOF mass spectrometer.

25. The method of claim 15, wherein said affinity capture probe has a chromatographic adsorption surface.

26. The method of claim 25, wherein said chromatographic adsorption surface is selected from the group consisting of reverse phase surface, anion exchange surface, cation exchange surface, immobilized metal affinity capture surface and mixed-mode surface.
